(19) United States
(12) Patent Application Publication
(10) Pub. No.: US 2025/0334577 A1
(43) Pub. Date: Oct. 30, 2025

(54) MARKERS FOR CANCER DETECTION (BF7)

(71) Applicant: MILAGEN, INC., EMERYVILLE, CA (US)

(72) Inventor: Moncef Jendoubi, San Francisco, CA (US)

(21) Appl. No.: 18/957,513

(22) Filed: Nov. 22, 2024

Related U.S. Application Data
(60) Provisional application No. 63/602,311, filed on Nov. 22, 2023.

(51) Int. Cl. G01N 33/574

(52) U.S. Cl. CPC G01N 33/57419

(57) ABSTRACT

Biomarkers can be assessed for a variety of uses, including screening, detection, diagnosis, prognosis, risk prediction, disease progression, recurrence, selection of treatment, therapy response, to evaluate a subject's health status, whether the subject presents with no evidence of disease, or a benign or malignant condition such as cancer. Compositions (antibodies, polypeptide and polynucleotide markers) and methods are provided herein, which find application in the early detection of cancer, in the early detection of disease relapse and in monitoring therapy response.

In accordance with 37 CFR 1.831 (2) a sequence listing is incorporated herein by reference. The sequence listing is entitled 2504-0004U, was created on Nov. 21, 2024, and is 47 KB.

### BACKGROUND

Over a million and a half estimated new cancer cases (1,638,910) in the US in 2012 caused over half a million (577,190) deaths. Over a lifetime, roughly half of all people between the ages of 50 to 70 will get some form of cancer. Cancer is the second leading cause of death after heart disease. The overall cost of cancer treatment exceeds half a trillion dollars and is constantly increasing.

One of the most important factors affecting the survival rate of all cancers is early detection. For many cancers, detection at the earliest stages yields survival rates greater than 90%, while detection at the later stages often causes survival rates to fall below 10%. In most cases, cancer is not detected until a proliferation of cancer cells is physically quite large, such as when an excess growth of tissue creates a lump or other mass that can be seen or felt by a cancer patient or when this mass causes pain or altered function in surrounding tissues or organs.

However, the earliest stages of cancer cause profound changes in the basic physiology of a patient, including changes at the genetic level. While excess cell growth itself causes fundamental changes, other physiological mechanisms are also affected when the cancer grows and spreads throughout the body. Changes in a cancer patients' DNA such as chromosomal alterations, alterations in gene sequences, and altered gene expression patterns also lead to modifications in protein expression. These changes in protein expression at the cellular level correlate with subtle changes in organs, tissues, and body fluids.

Although it is well recognized that a large number of proteins that are involved in the onset and development of cancer are fundamentally altered in terms of their structure, function, or expression, scientists have had limited success in identifying specific proteins that are uniquely associated with the development of cancer and are not found in normal patients. If such proteins could be reliably identified, detection of the proteins would be a valuable tool for the early detection of cancer leading to increased cancer survival rates in the entire population.

Where a particular protein is expressed only in cancer patients, or is expressed in a unique chemical form, or has any other distinguishing feature that distinguishes normal from cancer patients, such a compound may be called a “cancer marker” or “biomarker.” For many years, doctors and scientists have searched for cancer markers that uniquely identify the earliest onset of cancer. Ideally, these markers would not be present in other diseases or in benign conditions such that detection of such a marker would provide a reliable indicator that patient was in the earliest stages of developing cancer. In addition to early detection, these markers could be used to determine a prognosis in a patient, to predict the risk of cancer or relapse, to monitor disease progression or recurrence, to predict a patient's response to surgery or chemotherapy, to assess the effectiveness of treatment, or support patient and clinician's decision making in determining the appropriate course of prevention, surveillance or treatment.

While several potential markers have been analyzed for early cancer detection, very few have actually reached the clinical setting. Recommendations for a number of cancer markers have recently been reviewed by the National Academy of Clinical Biochemistry (NACB) and the American Society of Clinical Oncology (ASCO) panels: in breast cancer (Duffy, 2009; Harris, 2007), colon cancer (Brunner, 2009), lung cancer (Stieber, 2006), prostate cancer (Lilja, 2009), pancreatic cancer (Goggins 2005; Locker, 2006; Duffy, 2010), ovarian cancer (Chan, 2009), and cervical cancer (Gaarenstroom, 2007). A great need remains for early detection cancer markers because many existing markers, such as CEA, CA-15, CA-19, and CA-125, are elevated only in advanced cancer stages.

Some tests have shown an ability to predict whether a tumor in a patient is particularly aggressive. However, these tests typically require a tissue sample taken by an invasive procedure, such as a biopsy from the tumor, for gene expression analysis. These tests are not capable, or practical, for use in early detection in patients having no current symptoms.

Moreover, where the performance of the marker in separating cancer from normal is not adequate, the marker would have no utility when applied to the general population. In other words, while a marker may be used in patients already diagnosed with cancer, or in those at high risk, the ideal marker would be able to reliably distinguish a normal patient from an early cancer patient with enough accuracy that the marker could be used to screen the generally healthy population for early detection of cancer.

Furthermore, while scientists who analyze cancer tissue can readily detect fundamental differences between tumor tissue and regular tissue, those differences are not always attributable to the cancer itself and may be the result of inflammation or other events or conditions that are not directly related to the early onset of cancer. Furthermore, the examination of cancer tissue is not a viable approach for the early detection of cancer in the general population. It is simply impractical, and would be overly burdensome and costly, to surgically remove tissue samples from the general population, even in those patients where a high risk of a tumor exists. Furthermore, the methods to detect cancer often involve expensive and potentially damaging analytical methods, such as x-rays and CT scans that cannot be routinely applied to the population at large and are reserved for only those cases where a clinical diagnosis is already made.

Therefore, an ideal cancer marker would satisfy several different criteria: 1) the marker would identify the onset of cancer at an early stage where the prognosis for a cure and long-term survival are the greatest, 2) the marker would distinguish between normal patients, or those with a benign condition, and early stage cancer patients with very high reliability and would yield limited false negative results, i.e. failing to detect the early development of cancer in patients who in fact have an early stage cancer, and would yield limited false positives, i.e. incorrectly identifying a patient with cancer who is actually cancer free.

Still further, an ideal marker for the early detection of cancer would be simple and inexpensive to detect and could be detected in a patient's body fluid such as blood or urine, such that the test could be performed without a biopsy to remove tissue or other invasive or expensive procedures. Also, an ideal marker could be measured as a simple laboratory test that is conveniently and routinely performed as part of a regular visit to the doctor.

Because a wide variety of blood tests and urinalysis are routinely performed in doctors' offices and medical laboratories, a test kit or method for the early detection of cancer would be a powerful addition to the existing battery of tests performed on patients as part of ordinary health management. Moreover, in patients who are at high risk of developing cancer, i.e. certain patients in the aging population or with a family history or other history indicating a high risk of cancer, the ability to detect and treat cancer at the earliest stages would save millions of lives and preserve billions of dollars in resources otherwise dedicated to treating late stage cancer.

Therefore, an urgent need exists for cancer markers for all types of cancer where the marker enables non-invasive early cancer detection methods, and where tests identifying the marker are accurate, reliable, sensitive and specific, and that can be applied to the asymptomatic general population. If such markers were identified, they could also be used to obtain a prognosis upon detection in the body, to track the progression or metastasis of cancer, to track the treatment response once surgical or drug therapy begins, to identify patients who are free of cancer and thus require regular annual screening, and those in need of more active surveillance.

In the specific case of colon cancer, 140,250 new estimated cases of colorectal cancer (CRC) in 2018, CRC is the third most common cancer in men and women in the US. With 50,630 estimated deaths, CRC represents 8% of all US malignancies, and is the second leading cause of cancer mortality in men and women (Siegel, 2018; ACS, 2018). Rates of CRC incidence and mortality in the US have decreased in the population>50 years of age, due to advances in CRC treatment, more effective screening, and life style changes in that age group (Siegel, 2017; ACS, 2018). However, an alarming trend has been reported in the <50 adult population where CRC incidence rates have increased by 22% and mortality rates by 13% from 2000 to 2013 driven solely by tumors in the distal colon and rectum (Bailey, 2015; Siegel, 2017).

As in any other cancer, early detection of CRC is key to survival. The 5-year survival rate for localized disease is 90% (ACS, 2018). However only 39% of US patients are diagnosed at this stage (ACS, 2018). Screening recommendations for average risk asymptomatic adults starting at age 50 include colonoscopy every 10 years and high-sensitivity fecal immunochemical test (FIT) annually, or the Cologuard multitarget stool DNA test every 3 years (ACS, 2018; USPSTF, 2016; Smith, 2017).

Among screening methods, colonoscopy is the gold standard as it detects CRC and allows polyp detection and removal as well. However, this imaging method requires bowel preparation and sedation, is costly and carries the risk of possible complications (ACS, 2018).

Among non-invasive methods, the fecal immunochemical test (FIT) uses antibody-based detection of hemoglobin in patient stool. It is safe, cost-effective, and easy (no bowel preparation, no dietary restrictions, at home sampling). FIT performance varies widely depending on brand, manufacturer, and hemoglobin cutoff used, with 79% sensitivity (SE) at 94% specificity (SP) based on a meta-analysis (USPSTF, 2016; Lin, 2016; Lee, 2014). However, FIT has a very poor detection of advanced adenoma around 24% SE at 95% SP (Lin, 2016; Robertson, 2017).

Cologuard the stool DNA test (sDNA, Exact Science, Madison, WI; Imperiale, 2014) is a complex test that uses 13 multi-target assays based on the detection of DNA methylation and mutational markers by quantitative PCR, as well as detection of occult hemoglobin by ELISA. The test claims 92.3% SE for CRC and 42.4% SE for advanced adenoma (AA) at 89.8% SP. Comparatively FIT performance yielded 73.8% and 23.8% SE respectively at 96.4% SP (Bailey, 2016; Imperiale, 2014). Although three major screening methods are available to patients, CRC screening compliance is low because of either the invasive procedure or the so-called “ick factor” (Pratt, 2014; CDC, 2013).

It should be noted that the well established serum tumor marker CEA has no clinical utility as early detection marker because it lacks sufficient sensitivity and specificity and most patients will present with CEA-negative disease at time of diagnosis (Duffy, 2001; Brunner, 2009). Instead, CEA is in clinical use for determining prognosis and monitoring therapy in advanced disease (Brunner, 2009).

The recent development of EpiproColon test (Epigenomics, Gaithersburg, MD) provides an alternative to fecal-based tests. EpiproColon is a serum assay based on real-time PCR detection of aberrant methylation in the SEPT9 promoter using circulating cell-free DNA (ccfDNA) isolated from patient plasma. Diagnostic performance yields 68% SE for CRC and 21% for adenoma at 81% SP (Potter, 2014). This test has been FDA approved in 2016 with the limitation that it “should be offered to patients who decline CRC screening methods according to appropriate guidelines”.

Circulating cell free DNA (ccfDNA) is among a new type of biomarkers generally referred to as “liquid biopsies” which involve the sequencing of tumor-specific genetic alterations present in circulating nucleic acids found in patient blood (Bettegowda, 2014; Brock, 2015). The term globally refers to circulating tumor cells (CTCs) or nucleic acids in patient blood, including tumor or cell-free DNA (cfDNA, ctDNA), RNA from exosomes (exoRNA), circulating miRNA, (microRNA; Chen, 2014), mRNA and long non-coding RNAs (lncRNAs). Liquid biopsies have generated great interest as potential source of biomarker discovery for cancer detection, monitoring and therapy response. So far limited results have been obtained using circulating DNA or microRNAs to develop novel biomarkers for CRC early detection (Shah, 2014; Yiu, 2016; Ogata-Kawata, 2014; Yan, 2017). While offering great potential, liquid biopsies still face technological challenges. Indeed, TCGA data show immense variation at the DNA, RNA and epigenomic levels among cancers, between patients and within patients (Aravanis, 2017; TCGAN, 2013; Guinney, 2015). Also, studies have used a variety of measurement techniques making comparisons difficult, and small sample sizes, while large data sets are needed.

Overall, lack of standardization, variability among assay platforms and cost limit the use of cfDNA in early detection and diagnosis (Coticchia, 2015; Buden, 2016). More promising results have been obtained in using ctDNA in monitoring metastatic breast cancer (Dawson, 2013), although cancer therapy induces mutations, making it difficult to differentiate mutations due to disease progression from those due to therapeutic intervention.

It is well established that the majority of CRCs derive from precursor lesions such as adenomas and that polypectomy decreases the incidence of CRC in the treated population thus reducing CRC mortality (Fleming, 2012; Zauber, 2012). Hence current clinical practice focuses on removing early polyps to prevent CRC. Current non-invasive methods (FIT, Cologuard, EpiproColon) have poor or limited performance in advanced adenoma (AA). Therefore, any competing technology in the CRC screening space needs to provide a non-invasive serum-based assay with high SE/SP not only for CRC but also for AA.

Therefore, an urgent need remains for a serum or blood-based, non-invasive, affordable, and easy-to-use in-vitro diagnostic assay (IVD) to complement and improve on current early detection methodologies.

Uptake of CRC screening and lifestyle changes in the >50 year old adult population have contributed to earlier detection of disease and reduced CRC mortality. Indeed, two-thirds of CRC patients undergo CRC resection with curative intent, and the 5-year survival rate for localized disease due to early detection is 90% (ACS, 2018). For all stages combined, the 5-year relative survival rate is 64% for colon cancer and 67% for rectal cancer (ACS, 2018). Improved early detection and treatments have translated into increased survival rates thus impacting the number of CRC survivors. It is estimated that there are 1.5 million CRC survivors in the US alone and that they will be 1.8 million by 2026 (Miller, 2016).

While survival rates have improved, patients still face relapse: the relapse rate remains close to 30-50% (Abulafi, 1994). Specifically, 80% of recurrence cases have been reported to occur within the first 2-2.5 years after surgery, and 95% occur by 5 years (Jeffrey, 2013; Moy, 2016). The early detection of relapse has become a critical priority for clinicians, as that increases the chance of further surgical resection and earlier adjuvant treatment, improving overall survival (Duffy, 2001, Fakih, 2006).

Given the current incidence of CRC in the population, and the increased survival rate due to improved early detection and treatments, surveillance has become a significant priority in the management of CRC patients. Cancer patients remain indefinitely at risk of recurrence, and methods are needed to detect early recurrence events in asymptomatic patients (prior to symptoms and detection by imaging procedures), and at a reasonable cost.

Current guidelines for CRC post-treatment surveillance in patients with stage II or III disease include physical examination and serum carcinoembryonic antigen (CEA) testing every 3 months for at least 3 years after diagnosis, with additional imaging, such as computed tomography (CT) and colonoscopy as appropriate (Lockey, 2006; Moy, 2016; Jeffreys, 2013).

Routine CEA testing is in wide clinical use despite marker limitations (Duffy, 2001; Brunner, 2006). Reported sensitivity of CEA in detecting relapse varies widely, from 41-97% (Nicholson, 2016). The recent FACS study gave sensitivity (SE) of 50% for single point CEA testing (Shinkins, 2017). Overall a review analysis of 52 studies concluded that CEA is insufficiently sensitive to be used alone in detecting recurrence in patients following curative resection, even with lower thresholds (Nicholson, 2016).

Furthermore 30-40% of all CRC recurrences are not associated with measurable elevations in serum CEA (Moy, 2016). Clinicians wishing to offset the disadvantages of a single marker test often use CA19-9 as an additional serum marker. However, CA19-9 performance is poor and ASCO guidelines do not recommend it as surveillance test due to insufficient data (Locker, 2006).

Multigene signature assays have been developed to predict the risk of CRC recurrence or distant metastasis after a primary diagnosis and to guide patient management in eligible patients such as OncotypeDx Colon Cancer (Genomic Health; Clark-Langone, 2010) and ColoPrint (Agendia; Kopetz, 2015) as reviewed (Kelley, 2011). In practice, these assays measure the expression level of a given gene signature in biopsied or resected patient tissues and yield an algorithm-based score. However the Oncotype tests requires fixed patient tissues, which generally are available from patients, while ColoPrint requires fresh tumor tissues, which may limit adoption of this assay in the clinical practice. Moreover, while these tissue-based assays predict the risk of recurrence, they are a one-time test, and insofar they are not geared to monitor recurrence over a period of time as a surveillance procedure.

Increasing amounts of data support the view that early diagnosis of recurrent disease results in a more favorable outcome (Moy, 2018; Bruinvels, 1994), reinforcing the notion of intensive surveillance, which commands simple and cost-effective methods to monitor the emergence of recurrence. Because of cost and limited access, intense imaging surveillance remains impractical. With respect to health care costs, CRC accounted for 14 billion dollars in cancer care in 2010, with annual costs exceeding those reported for breast and prostate cancer combined. Total cost for CRC care will reach over 17 billion in 2020 (Mariotto, 2011). Thus there is a clear need for new cost-effective tests that can complement current surveillance practice and contribute to improve the overall quality of care of CRC patients.

A simple serum-based test in combination with CEA would be advantageous to enable earlier detection of disease relapse. Such test would most likely save patients the secondary effects of systemic therapies and increase their long-term survival while improving overall quality of life.

Therefore, biomarkers that can accurately monitor the emergence of a recurrence in asymptomatic patients prior to imaging would improve on current surveillance procedures. Detecting a biomarker change in a patient which is associated to recurrence, prior to appearance of symptoms and imaging results would save the patients the toxic secondary effects of systemic therapies and increase their long-term survival while improving overall quality of life.

### SUMMARY OF THE INVENTION

The invention encompasses compositions and methods for the early detection of colorectal cancer, for the early detection of disease relapse and for monitoring therapy response.

By practicing the method steps protein cancer markers are measured in a biological fluid sample in order to determine with high sensitivity and specificity the presence of CRC in a patient and preferably early stage CRC. The results inform the clinician on whether the patient requires further treatment, further exploratory procedures, active follow-ups or regular recommended screening. The detection methods may further be comprised of additional known markers measured by any technique. For monitoring disease relapse a number of known options can be combined with the methods and assays of the present invention to inform the selection of further treatment, further exploratory procedures or intervention.

The compositions of the present invention include assays, reaction mixtures, and analytical systems wherein a monoclonal antibody as defined herein, namely BF7, binds a collection of polypeptides disclosed herein at specific binding sites. The amino acid sequence on the polypeptide at the antibody binding site is called “epitope”. The epitopes of the polypeptides disclosed herein share common features and amino acid functionalities. Sequences with homology to the epitope or binding motif (e.g. mimotopes) are also disclosed herein. Measurement of the collection of proteins is achievable by known methods for quantifying proteins in a reaction mixture. To that end, the antibody may also contain markers or other detection moieties for localization of the antibody or the detection of the binding of the antibody to the binding motif on the markers, to allow quantitative measurement of the collection of polypeptides in the reaction mixture that have formed an immunological complex (antigen-antibody) at the target epitopes.

The core of the invention is the non-human non-naturally occurring monoclonal antibody BF7, capable to bind the collection of polypeptides disclosed herein, thus enabling the detection of protein cancer markers that alone, in combination with at least another marker, or collectively ultimately provide discrimination between CRC patients and healthy controls.

The protein markers recognized by the BF7 antibody are identified herein by their name, standard abbreviation and amino acid sequence, along with the nucleotide sequence encoding the polypeptide sequence of each marker. Indeed, encompassed in the compositions of the present inventions are the nucleotide sequences, as well as synthetic gene constructs, encoding the protein markers.

The detection also includes detecting non-natural variants of each of the foregoing in any assay format. The format for detection of the protein markers is not critical to utility of the invention and the markers, whether in the form of polypeptides or polynucleotides, and related species as defined herein can be detected by any existing technique known in the art for accurate identification of a polypeptide or polynucleotide sequence, or synthetic constructs based thereon, in a biological or patient sample, in addition to the immunoassay method described herein.

Because the protein markers are secreted from the cells of a human patient into a “biological fluid” or “patient test sample”, typically the blood or urine of the patient, the detection of the markers using conventional assay platforms for analysis of blood and urine is included within the invention. Identification of the markers also enables the detection of autoantibodies where present. The antibody described below for binding the markers may be used in any laboratory test format that uses a binding reaction between the polypeptide markers and the antibody to determine the presence of the markers in a biological sample.

The detection of the markers, the antibody, the genes or related species such as pre-RNA, mRNA, etc. can take place in an in vitro diagnostic kit for detection of cancer in a biological sample, or in a patient test sample, and in a large scale, high throughput format assay method or system for processing large numbers of samples.

The invention also includes methods for detecting the polynucleotides, pre-RNA, mRNA, or any species associated with transcription of the polynucleotides disclosed herein, or any species associated with the translation process yielding the markers. Also, the polypeptide markers may be transformed into a derivative or synthetic construct useful for detection or for creating novel or engineered antibodies for detection of the markers or a variant thereof. Also additional methods for using other antibodies, different from the one monoclonal antibody described herein, that are specific for the markers or variants thereof, are enabled.

The methods of the invention include measurement or detection of any component of the polypeptide markers including fragments, modifications, post translational modifications, truncations, or essentially any adequate representative sample of an amino acid sequence of which the polypeptide markers are comprised to determine the presence of the polypeptides in a sample. This includes using novel antibodies (polyclonal, monoclonal, Fab fragments, etc.) enabled by the description below to separate the markers described herein from a biological sample, such as a patient test sample in a test format wherein secreted proteins are identified. The monoclonal antibodies described herein can also be used in a diagnostic method to manufacture a new composition comprised of a complex of the novel monoclonal antibody and the markers. The methods also include distinguishing expression or secretion of the markers from other isoforms or variants of the markers, particularly where the detection events indicate the presence or progression of cancer or prognosis for, or response to treatment.

Specific uses of the methods described herein include detection of early cancer in the asymptomatic general population, detecting cancer in a suspect patient population having a high risk of developing cancer, tracking the status or progression of cancer in a patient, including the efficacy or success of a course of treatment over time by sequential measurement of the markers in a patient, preferably by secretion into a body fluid, but also including through measurement or analysis of gene expression or in tissue marker detection following a biopsy or imaging event. Similarly, by tracking the markers across a single patient over time, or through a population of patients at a fixed point in time or across numerous time periods, the efficacy of a new cancer treatment may be assessed. For example, where a new cancer therapeutic compound is under investigation, sequential measurements of the presence or quantity of the markers in a patient or a patient population provides an indication of the therapeutic utility of the clinical candidate.

The methods of the invention include detecting the markers described herein in a patient at a first time, at a second time, and at any number of times or discrete intervals occurring over time. The individual detection events can be part of a baseline monitoring procedure in asymptomatic patients or may be before during or after treatment for primary cancer, wherein changes in marker levels are indicative of disease presence, progression, relapse thus informing clinicians on the course of appropriate patient treatment, such as continue, stop or change therapy, pursue active surveillance with follow-up at regular intervals of time, confirm or exclude suspicious clinical status with further procedures, identify the stage of a particular cancer, recommend imaging or other diagnostic intervention, correlate the biological fluid based quantitative measurement with other accepted disease management procedures for colorectal cancer-including colonoscopy, and detection of other markers including CEA, CA-19 and others.

The methods of the invention include the techniques and protocols specifically used for testing the asymptomatic general patient population for cancer, diagnosing a patient or groups of patients, and the practice of predictive medicine, including where specific populations of patients are identified and tested for the early development of cancer. These specific or pre-determined populations can be defined by age, sex, ethnic origin, prior disease, family history, genetic markers (such as Her-2, BRCA 1/2), exposure to toxins, carcinogens, or environmental or other cancer risk factors, or any event that places a patient in a defined or higher risk population.

The invention provides methods of determining or predicting effectiveness or response to a particular treatment, monitoring patient response to therapy, and methods of selecting a cancer treatment for an individual. For example, markers that are differentially expressed by cells (e.g., cancer cells) that are more or less responsive (sensitive) or resistant to a particular cancer treatment as measured over time using the compositions and methods described herein for determining or predicting effectiveness or response to the treatment or for selecting a treatment for an individual.

Finally, the invention includes methods to detect cancer in an individual by measuring amounts of circulating or secreted markers in a biological or patient test fluid, such as in urine or serum, by immunological methods, comparing a quantitated or measured value to a reference value, and assigning to the sample a “most likely disease”, “most likely non-disease”, or “suspicious” diagnosis. Similarly the invention includes methods to monitor disease progression in a patient by measuring amounts of circulating or secreted markers in a biological patient test fluid, such as urine or serum, by immunological methods, over time by comparing a quantitated or measured value to a reference value, repeating that measurement for at least a second measurement and comparing to a reference value.

### DESCRIPTION OF FIGURES AND TABLES

FIG. 1: BF7 Mimotopes

Binding of individual phages, displaying 12-mer peptides BF7-3, BF7-15, and BF7-5 (SEQUENCE ID NO: 29 to SEQ ID NO: 31) to monoclonal antibody BF7 of the CRC antibody panel of the present invention, by ELISA. Phage clones with the best binding activity are amplified and sequenced. The amino acid sequence of the 12-mers from the three best binders is provided. These sequences are referred to as mimotopes to indicate that they comprise amino acids displaying homology to the actual BF7 epitope and are used to identify the collection of polypeptides described herein.

FIG. 2: BF7 Assay Characteristics

ELISA assay is described in detail in Example 9. The sandwich ELISA assay used matched capture and detection monoclonal antibody pair in standard buffer. Monoclonal antibodies were protein A/G purified from hybridoma culture supernatant or mouse ascites, and were used at the optimal concentration yielding the maximum signal to noise ratio with an acceptable non specific binding of OD<0.2. Serum-free cell culture medium (SFCM) from the breast cancer cell line MCF-7 was used as antigen source, and serially diluted in standard buffer. BF7 standard immunoassay protocol involved coating a 96-well plastic tray with capture monoclonal antibody, blocking unoccupied sites, incubating with surrogate antigen, binding captured BF7 polypeptides with biotinylated detecting monoclonal antibody, amplifying detection with streptavidin linked horseradish peroxidase (HRP) and revealing colorimetric reaction with HRP substrate as measured at 450 nm by microplate reader. Each data point was in triplicate, and BF7 polypeptide concentration equivalent was interpolated from the standard curve generated by using the 4 parameter logistic method.

Panel A. Typical antigen calibration curve for the BF7 antibody is shown. Stock antigen source (SFCM from the colon cancer cell line SW116, Example 9) was serially diluted in normal serum 1:10 or 1:20, and used to run ELISA assay with monoclonal antibody BF7, as described in detail in Example 9. Individual lines represent 6 independent calibration curves (standard deviation (SD) is indicated by a bar) performed on 3 separate days. Each data point is the mean of duplicate determinations. The table summarizes the average measurements of the same antigen sample (high or low CRC patient serum pool) in duplicate and over 4 days using the BF7 assay. Antigen levels are provided in microEquivalent/ml (μgE/ml). The standard deviation (SD) and the inter-assay reproducibility (% CV) are indicated. % CV=SD/mean*100.

FIG. 3: BF7 Diagnostic Performance

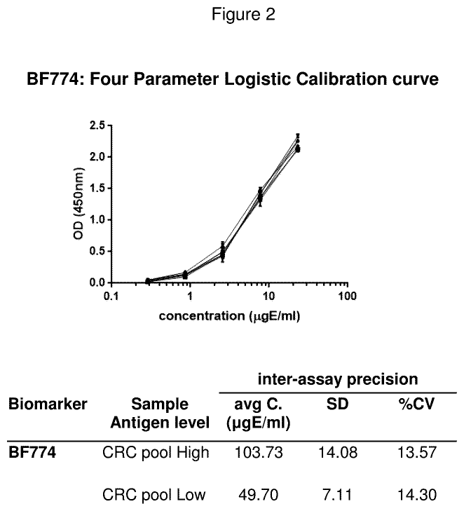

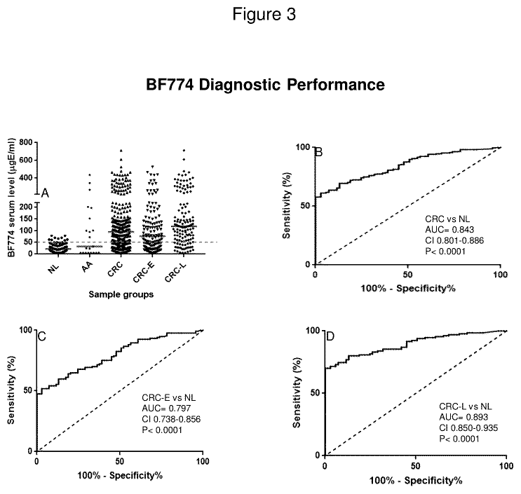

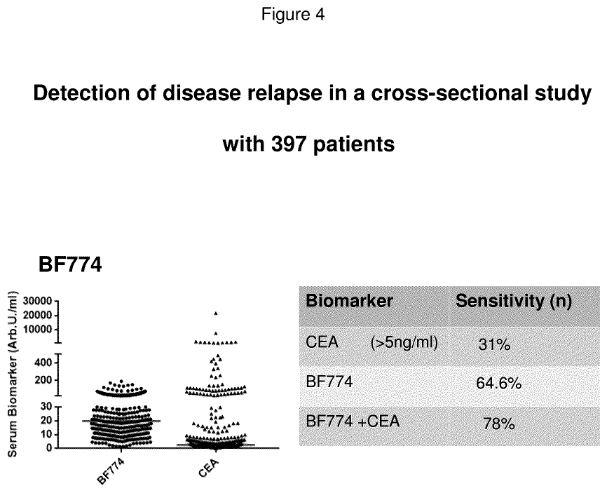

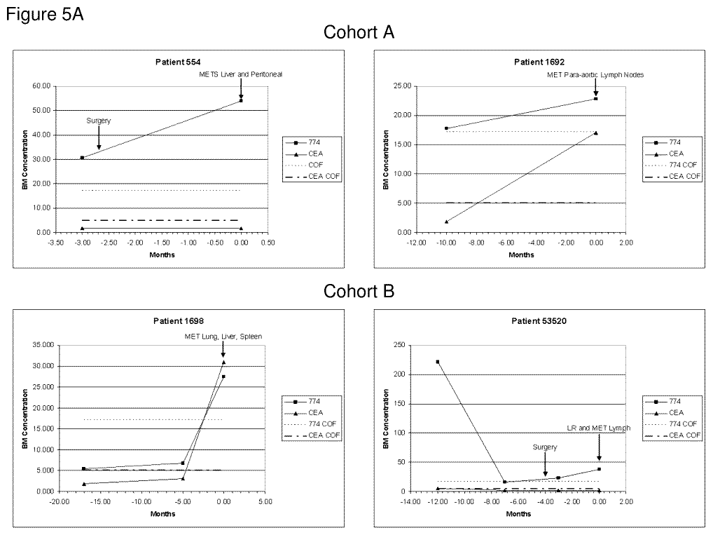

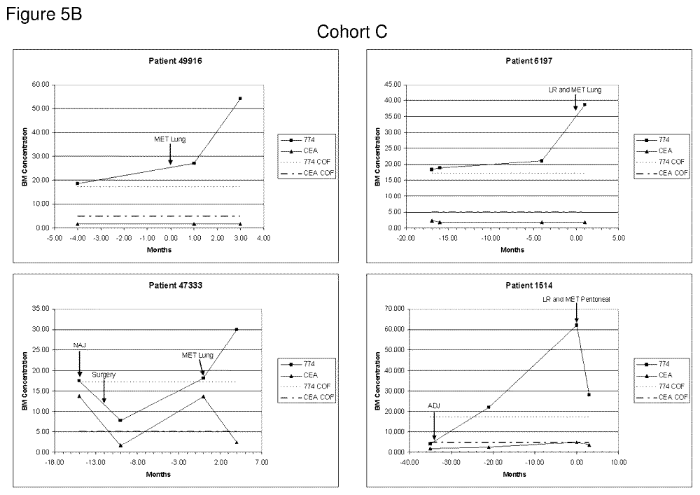

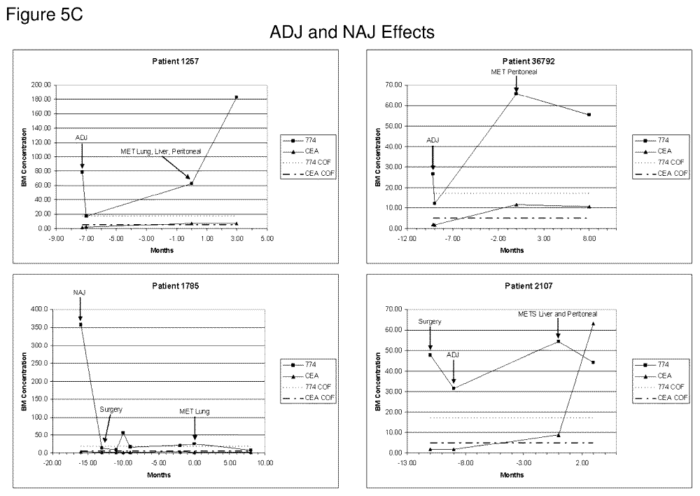

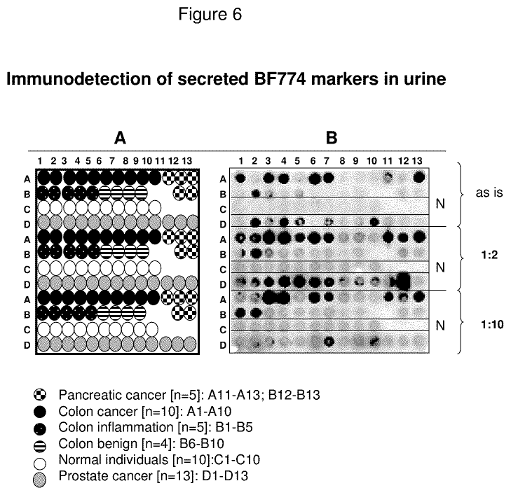

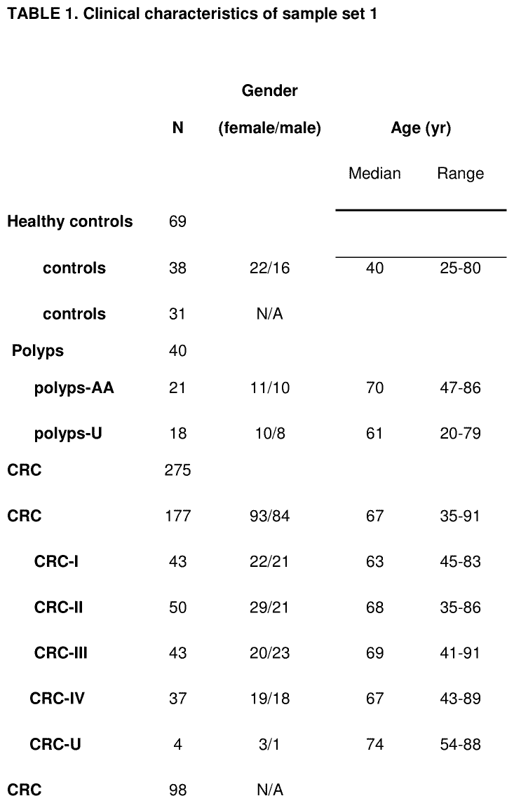

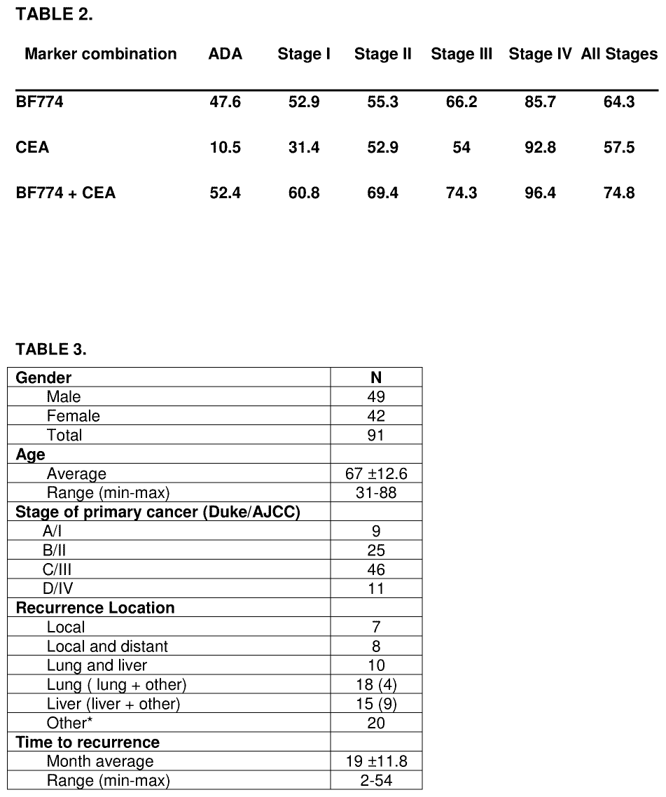

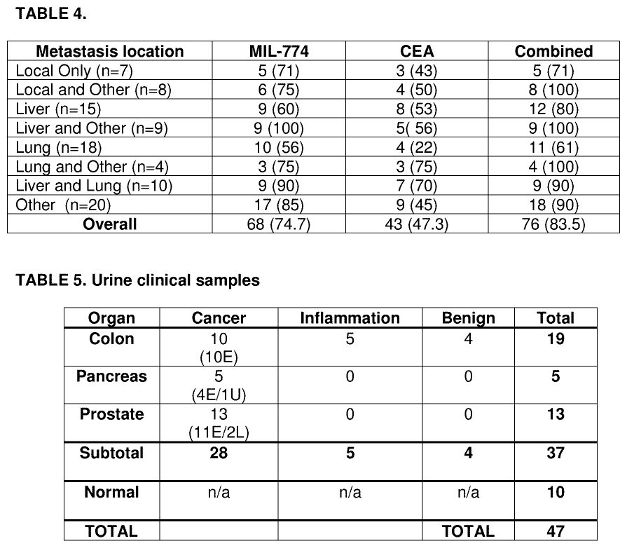

BF7 marker levels were measured in duplicate by sandwich ELISA assay in a training set of 384 serum samples including colorectal cancer cases (CRC; n=275), early (CRC-E; n=137) and late (CRC-L; n=130) stage, 40 polyps (of which 21 advanced adenoma (AA) and 19 polyps with unknown size and further histopathological data) and normal controls (NL; n=69; Clinical Sample Set 1, Table 1). Biomarker concentration was derived from the biomarker standard curve (FIG. 2) using 4-parameter logistic method.

Panel A: Scatter Plot. The median biomarker concentration values (illustrated by a solid line) of each group were compared to determine whether differences were statistically significant (p<0.05). Statistical analysis of two-group and multi-group comparisons was carried out using the Kruskal-Wallis test, followed by the Dunn's multiple comparisons test, as appropriate. Scatter plot of all sample intensity values illustrates the distribution and median difference of the CRC biomarkers among groups. BF7 median values in each group, represented by solid lines, are: 22.41 μgE/ml (NL), 32.3 μgE/ml (AA), 96.29 μgE/ml (CRC), 75.77 μgE/ml (CRC-E), and 119.1 μgE/ml (CRC-L). BF7 cutoff that discriminates CRC from NL at optimum SE/SP is indicated by a dotted horizontal line and is 50.6 μgE/ml.

Panel B-D: Receiver Operating Characteristics (ROC) curves were calculated to evaluate BF7 diagnostic performance by determining sensitivity, specificity, area under the curve (AUC), and 95% confidence interval (CI) in the following comparisons: “CRC versus NL” (panel B); as well as “early stage CRC-E versus NL” (panel C) and “late stage CRC-L versus NL” (panel D). Statistical analysis, ROC curves and scatter plots were performed with GraphPad Prism7.0. The chance line is indicated by a dotted line.

FIG. 4: Detecting disease relapse

Scatter plot comparison between BF7 and CEA in the detection of recurrent and metastatic disease in 397 patients in a cross-sectional study: a cutoff of >5 ng/ml for CEA and >17 μgE/ml for BF7 (specificity of 82% as established on 40 normal controls) was used. The table indicates sensitivity for each biomarker alone and in combination. BF7 levels were the average of duplicate measurements. CEA measurements were obtained from measurements at patient hospital visit.

FIGS. 5A-5C: Monitoring patient follow-up

Individual patient charts illustrate patient follow-up using the BF7 assay. Serial serum samples from 12 patients after a primary colorectal cancer were measured and test results were plotted versus months of follow-up. Negative months refer to time prior to diagnosis of recurrence. Any available indication of disease relapse and treatment event (chemotherapy, radiation, metastasis at different sites) is indicated. Abbreviations: neoadjuvant therapy (NAJ); adjuvant therapy (ADJ); locoregional recurrence (LR); distant recurrence/metastasis (MET).

FIG. 6: Immunodetection of secreted BF7 markers in urine

Panel A: Diagram illustrating the spot location of 47 urine samples from patients with: pancreatic cancer (n=5; 4 stage II and 1 unknown), colon cancer (n=10; all stages I or II), colon inflammatory diseases (n=5), colon benign conditions (n=4), prostate cancer (n=13; all stage II except D9 and D10 of stage III) and normal controls (10). Note that cancer stages I and II are defined as “early”, while stages III and IV are defined as “late”.

Panel B: Total urine proteins after centrifugation to remove debris, were spotted on the MPAT membrane in a blind experiment, in three different conditions: as is, or diluted 1:2 or 1:10 in Tris-Triton buffer as indicated. Protein samples were then incubated with BF7 monoclonal antibody and immunodetected as described in Example 13.

Table 1: Clinical Characteristics of Sample Set 1

Clinical sample set 1 comprises: 275 CRC, 40 polyps and 69 healthy controls. The table shows the breakdown of samples by type (CRC, polyps, and healthy controls) and stage. Age median and range for each group is indicated, when available. Note that 98 CRC cases and 31 controls lacked age and gender information. Polyps were classified as advanced adenoma (AA) and polyps of unknown type (P-U), as size and other histopathological information were not available.

Table 2: Summary of BF7 Diagnostic Performance

Sensitivity (%) of the BF7 markers and CEA, alone or combination, in detecting CRC (n=275), whether per stage or all stages, and AA (n=21) versus NL controls (n=69) in Clinical Sample Set 1. The cutoff for BF7 markers, CEA or the combination was selected in order to yield 90% specificity based on NL controls. Then a sample was called positive whenever marker levels were above the cutoff (or any of the marker was above cutoff in the case of a combination).

Table 3: Patient clinicopathological data in Clinical Sample Set 3

The Clinical pathological features of patient population: n=91. Age average (years) and time to recurrence form primary surgery (months)+standard deviation are indicated. Other distant metastasis sites include: lymph nodes (axillary, para aortic), peritoneum, brain, kidney, ovary, skeleton, stomach.

Table 4: Breakdown of Sensitivity by Location of Metastasis

Number of patients and % SE (indicated in parenthesis) with elevated BF7 and elevated CEA. Cutoff values of 17.1 μgE/ml BF7 and 5 ng/ml CEA were used. Serum marker was considered to be in positive concordance with clinical diagnosis of recurrence if above cutoff at the sample point closest to recurrence. Other distant metastasis sites include: peritoneal, para-aortic lymph nodes, brain, kidney, ovary, skeleton, lymph nodes.

Table 5: Urine clinical samples

Composition of the clinical sample set used to detect the presence of polypeptides or fragments thereof bearing a BF7 binding motif in urine of cancer patients, benign and normal controls, as illustrated in FIG. 6. Details on disease stage and inflammatory conditions are provided in FIG. 6 and its legend. E: early stage cancer (stages I and II); L: late stage cancer (stages III and IV); U: unknown.

### DETAILED DESCRIPTION OF THE INVENTION (BF7)

The present invention relates to composition and methods using a novel, non-human non-naturally occurring monoclonal antibody designated BF7 that recognizes biomarkers whose polypeptide sequence is listed herein (SEQ ID NO: 1 to SEQ ID NO: 31) which find application in the early detection of cancer, in the early detection of disease relapse, and in the monitoring of therapy response. These markers, which have in common and are defined by a BF7 binding motif, are collectively and interchangeably referred to herein as “BF7 markers”, “BF7 biomarkers”, “BF7 polypeptides” or “BF7 proteins”. The use herein of the plural “markers”, “biomarkers”, “polypeptides” or “proteins” refers to any single species as well as to any combination thereof (of 2, 3, 4, or more). Specifically, any combination of 2, 3, 4, 5, 6, 7, 8, 9, 10, 11, 12, 12, 13, 14, 15, 16, 17, 18, 19, 20, 21, 22, 23, 24, 25, 26, 27, 28, 29, 30, 31 proteins is contemplated by the present invention. The present invention discloses that BF7 markers are differentially expressed (over-expressed) in individuals with cancer as compared to individuals without cancer (individuals without cancer are interchangeably referred to herein as “normal”, “control”, or “healthy” individuals). The present invention revolves around the use of BF7 monoclonal antibody that provides a tool to discriminate with high accuracy patients with cancer, and preferably patients with early stage colorectal cancer.

BF7 monoclonal antibody may be used in a variety of clinical indications for cancer, including, but not limited to, detection of cancer (such as in an asymptomatic individual or population or in a high-risk individual or population), characterizing cancer (e.g., determining cancer type, sub-type, or stage) such as distinguishing between CRC and benign tumors using BF7 for imaging, cancer prognosis, monitoring cancer progression or remission, monitoring for cancer recurrence or metastasis, selecting treatment, monitoring response to a therapeutic agent or other treatment, stratification of patients for MRI or computed tomography (CT) screening (e.g., identifying those patients at greater risk of cancer and thereby most likely to benefit from enhanced screening, thus increasing the positive predictive value of any parallel screening method), combining testing of BF7 markers with supplemental biomedical parameters and patient clinical information, such as those listed in Table 2, or such as toxin exposure, smoking history, known or suspected presence of hereditary syndromes such as Lynch syndrome (hereditary non-polyposis colon cancer) of familial adenomatous polyposis (or any other mutations cited in Jasperson, 2010) or any of the existing markers noted below, or with tumor or nodule size, tumor morphology, etc. (such as to provide an assay with increased diagnostic performance compared to another testing technique alone or in combination with BF7, facilitating the diagnosis of a biological sample as malignant or benign, facilitating clinical decision making once a cancer is observed by margins, or of biopsy if the sample is deemed medium to high risk, and facilitating decisions regarding clinical follow-up (e.g., whether to implement repeat detection of this or another marker, imaging, biopsy, or other measure).

BF7 markers may be quantified when diagnosing cancer such that a high or low abundance level in an individual who is not known to have cancer may indicate that a threshold amount present in a sample from the individual correlates to cancer at a specific stage, thereby enabling early detection of cancer at an early stage of the disease when treatment is most effective, i.e. perhaps before the cancer is detectable by other techniques or before other symptoms appear. An increase in the abundance of BF7 markers may be indicative of cancer progression, e.g., a tumor or abnormal tissue is growing and/or metastasizing (and thus a poor prognosis), whereas a decrease in the abundance of BF7 markers may be indicative of cancer remission, e.g., a tumor is shrinking (and thus a good prognosis). Similarly, an increase in the abundance of BF7 markers during the course of cancer treatment may indicate that the cancer is progressing and therefore indicate that the treatment is ineffective, whereas a decrease in the abundance of BF7 markers during the course of cancer treatment may be indicative of cancer remission and therefore indicate that the treatment is working successfully. Additionally, an increase or decrease in the abundance of BF7 markers after an individual has apparently been cured of cancer may be indicative of cancer recurrence or metastasis. Detection of “differential” expression, or variation from a “normal” expression level, can also be used for another purpose described herein.

Detection of BF7 markers may be particularly useful following, or in conjunction with cancer treatment, such as to evaluate the success of the treatment or to monitor cancer remission, recurrence, and/or progression (including metastasis) following treatment. Cancer treatment may include, for example, administration of a therapeutic agent to a patient, surgery (e.g., surgical resection of at least a portion of abnormal tissue or a tumor), radiation therapy, or any other type of cancer treatment used in the art, and any combination of these treatments.

BF7 monoclonal antibody and antibodies to BF7 markers may also be used in imaging tests. For example, an imaging agent can be coupled to BF7 antibody which can be used to aid in cancer screening or diagnosis, to monitor disease recurrence, progression/remission or metastasis, to plan surgery, biopsy, or radiation therapy, or to monitor response to therapy, among other uses. The BF7 monoclonal antibodies disclosed herein are formulated to enhance stability, reduce immunogenicity and enhance plasma half-life, pH-range stability and other desirable pharmacological parameters by techniques known in the art.

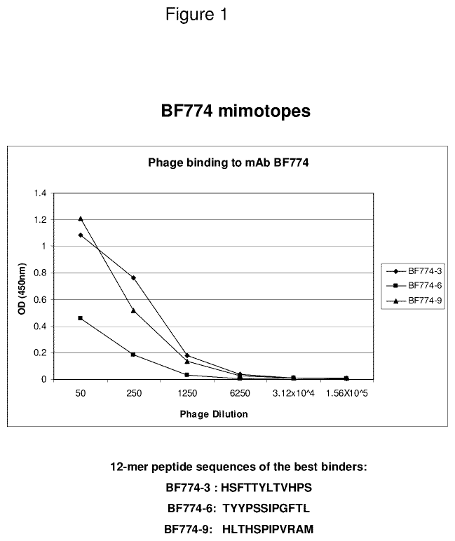

As used herein the term “antibody” refers to a polyclonal, monoclonal, recombinant antibody, full-size molecule or antibody fragment thereof, including but not limited to Fab′″, scFv, single chain variable fragment, affibodies, diabodies, or any other antibody fragment, or any other recombinant version of conventional or combinatorial antibody, as well as any single or double chained binding agents comprised of a variety of known structures, including another molecule or biologically compatible tag that facilitates detection of the antibody while retaining the ability of the antibody to recognize the relevant epitope (such as the mimotopes of FIG. 1) or the common characteristics of the BF7 binding motif to a sufficient extent for detection to occur.

Unless specified, the term “antibody” is used interchangeably herein to refer to any of the above species. The novel compositions are comprised of non-naturally occurring species of monoclonal antibodies capable of binding BF7 markers. Methods of the invention include use of both naturally occurring and synthetic variants of BF7 antibody. Thus, “antibodies” include antibodies produced in vitro, as well as antibodies generated in vivo by injection of BF7 markers or polynucleotides encoding BF7 markers in a mammal capable of mounting a sufficient immune response to yield high titre IgG antibodies. Methods to produce polyclonal, monoclonal, recombinant antibodies and fragment thereof are known to the skilled in the art (Coligan et al., Current Protocols in Immunology, Wiley Intersciences; Kohler et al. Nature 256:495-497, 1975; Phage display of peptides and proteins-A laboratory manual, Kay B. B., Winter J. & McCafferty J., Eds, Academic Press, 1996).

The term “monoclonal antibody”, as used herein, refers to a novel antibody obtained from a population of substantially homogeneous antibodies, i.e., the individual antibodies comprising the population are substantially identical except for naturally occurring mutations present in minor amounts. Monoclonal antibodies are highly specific and are typically directed against a single epitope and variants thereof as described below, in contrast to polyclonal antibody preparations which typically include different antibodies directed against different determinants (epitopes). In addition to specificity, the monoclonal antibody against BF7 markers described herein is substantially homogenous and is produced by an available hybridoma. The modifier “monoclonal” indicates that the antibody exists in a substantially homogeneous population of antibodies, but is not to be construed as requiring production of the antibody by any particular method.

An “isolated” or “purified” antibody is one that has been identified and separated and/or recovered from a component of the environment in which it is produced. Contaminant components of its production environment are materials that would interfere with diagnostic or therapeutic uses for the antibody, and may include enzymes, hormones, and other proteinaceous or nonproteinaceous solutes. In exemplary embodiments, the antibody can be purified as measurable by any of at least three different methods: 1) to greater than 95% by weight of antibody as determined by the Lowry method, preferably more than 99% by weight; 2) to a degree sufficient to obtain at least 15 residues of N-terminal or internal amino acid sequence by use of a spinning cup sequenator or 3) to homogeneity by SDS-PAGE under reducing or non-reducing conditions using Coomassie blue or silver stain. Isolated antibody can include an antibody in situ within recombinant cells since at least one component of the antibody's natural environment will not be present. Ordinarily, however, an isolated antibody can be prepared by at least one purification step.

The term “non-human” is used to distinguish naturally occurring antibodies existing in a human host that may be reactive with a target antigen as described herein. Non-human antibodies are produced in a non-human host species and have chemical or structural signatures characteristic of the host that do not exist in the target species. An example is a “non-human” BF7 antibody to the human BF7 proteins produced in a bacterial species such as an E Coli or in a mammalian species such a CHO cell having glycosylation or other chemically distinct signatures compared to a BF7 antibody existing or produced in a human. With the development of humanized antibodies and antibodies produced in transgenic animal species having human variable and/or constant regions a ‘non-human” antibody may not be distinguished by amino sequence but by the expression or production in a non-human species.

“Antibody specificity” refers to an antibody that has a stronger binding affinity for BF7 polypeptides from a first individual species than it has for a homologue of BF7 from a second species. Typically, a BF7 antibody “binds specifically” to a human BF7 antigens (e.g., has a binding affinity (Kd) value of no more than about 1×10-⁷ M, preferably no more than about 1×10⁻⁸ M, and most preferably no more than about 1×10-⁹ M) but has a binding affinity for a homologue of the antigen from a second individual species at least about 50-fold, or at least about 500-fold, or at least about 1000-fold, weaker than its binding affinity for the human BF7 polypeptides.

An antibody “selectively” or “specifically” binds the BF7 marker proteins when the antibody binds the marker proteins and does not significantly bind to unrelated proteins, lacking the BF7 binding motif, for example. An antibody can still be considered to selectively or specifically bind a marker protein even if it also binds to other proteins that are not substantially homologous with the marker protein as long as such proteins share substantial homology with a fragment or domain of the marker protein epitope. Antibody binding to the marker protein is still selective and “specific” despite some degree of cross-reactivity to other antigens.

The term “epitope” is used to refer to the amino acid sequence within the BF7 marker polypeptides recognized by the BF7 antibody disclosed herein. The term “epitope”, “antigenic determinant”, “structural domain”, “antibody target” or “binding motif” are interchangeably used to indicate the amino acid sequence, whether in isolated form or embedded in a polypeptide sequence or fragment and derivative thereof, which is recognized by the BF7 antibody. Epitopic determinants can be active surface groupings of molecules such as amino acids or sugar side chains and may have specific three-dimensional structural characteristics or charge characteristics. Preferably, we will refer herein to the common characteristics of the binding motif, recognized by the BF7 monoclonal antibody and shared by the BF7 protein markers, to exemplify the well known notion in the art of antibody-antigen interactions whereby an antibody can bind to different polypeptide sequences sharing epitope homology, as described above. Said homology includes amino acid changes, preferably involving conservative amino acid substitutions, yet also including any amino acid substitution that maintains binding functionality, such as permutations, deletions, or insertions. As a result, alterations to the sequence of the epitope may exist as long as the BF7 antibody retains binding specificity as determined by the ability of the BF7 antibody to bind the BF7 markers at the altered epitope to form a complex in such a way that the binding event is detectable. Such altered sequences of the epitope are also referred to as “mimotopes” to indicate that they comprise sequences displaying homology, yet not identity, to the epitope, thus mimicking epitope functionality.

Therefore encompassed herein are binding agents other than an antibody as defined herein, such as but not limited to aptamer, peptide nucleic acid (PNA) as well as a polymer, solid support or chemical scaffold displaying anti-BF7 mimotopes or binding motif enabling the detection of the BF7 compositions.

The terms “natural polynucleotide”, or “natural nucleotide sequence”, are used interchangeably herein and may include naturally occurring DNA sequences or downstream transcripts such as pre-RNA. The “natural polynucleotide” described herein is DNA, including genomic DNA, double or single-stranded, whether coding or non-coding strands, or RNA, including heteronuclear RNA, messenger RNA (mRNA), or any other form of RNA, such as small, anti-sense, interfering or silencing RNA whose expression correlates to the presence or expression of BF7 markers in vivo or in a biological sample.

A “synthetic polynucleotide” or “polynucleotide construct” may contain introns, 5′ and 3′ non-coding sequences, 5′ and 3′ transcriptional regulatory sequences, such as promoters, enhancers, polyadenylation signals, or translational control elements not present in the natural polynucleotides encoding the polypeptide markers as expressed in a human patient. The synthetic polynucleotides may include “natural polypeptide” sequences for BF7 markers that are manufactured to include DNA constructs to facilitate expression or regulation or that encode for leader or secretory sequences at the level of the polypeptides, or for active or inactive pro-proteins that are later processed into active or inactive shorter polypeptides. The assembled synthetic constructs are constructed and oriented to facilitate expression of the natural polypeptide in a non-natural environment.

The synthetic polynucleotides described herein include engineered splice variants and non-naturally-occurring allelic variants, and any non-natural variants encoding the biomarkers of the present invention with a different nucleotide sequence due to the degeneracy of the genetic code. Variants encode fragments, analogs and derivatives of the markers, and may include deletion, substitution, addition or insertion variants created by design even if duplicated by unusual and rare phenomena including those created. The synthetic polynucleotides encompassed by the claims include any length of said polynucleotide sequences, whether 5′ terminal, 3′ terminal or internal and transformed into entities chemically suited for use in a diagnostic platform.

Synthetic polynucleotides of the present invention, including DNA constructs can be manufactured using standard molecular biology techniques and the sequence information described herein (Sambrook et al., 1989, Molecular Cloning: A Laboratory Manual, 2^{nd} ed., Cold Spring Harbor Laboratory Press, Cold Spring Harbor, NY).

The BF7 protein markers are substantially free of cellular material or free of chemical precursors or other chemicals. BF7 proteins can be purified to homogeneity or other degrees of purity. The level of purification can be based on the intended use. The primary consideration is that the preparations allow for the desired function of the proteins, even if in the presence of considerable amounts of other components.

To determine the percent identity of two amino acid sequences e.g., a BF7 polypeptide targeted by the BF7 monoclonal antibody and a BF7 polypeptide variant thereof, the two sequences can be aligned for optimal comparison purposes (e.g., gaps can be introduced in one or both of a first and a second amino acid or polynucleotide sequence for optimal alignment and non-homologous sequences can be disregarded for comparison purposes). In an exemplary embodiment, at least 30%, 40%, 50%, 60%, 70%; 80% or 90%, 91%, 92%, 93%, 94%, 95%, 96%, 97%, 98%, 99% or 100% of the length of a first sequence can be aligned for comparison purposes with a second sequence. The amino acid residues or nucleotides at corresponding amino acid positions or nucleotide positions are compared and relative functionality analyzed by techniques known in the art. When a position in the first sequence is occupied by the same amino acid residue or nucleotide as the corresponding position in the second sequence, then the molecules are identical at that position (as used herein, amino acid or nucleic acid “identity” is equivalent to amino acid or nucleic acid “homology”). The percent identity between the two sequences is a function of the number of identical positions shared by the sequences, taking into account the number of gaps, and the length of each gap, that are introduced for optimal alignment of the two sequences.

The monoclonal antibody disclosed herein is monoclonal antibody BF7 and is preferably identified by its ability to recognize a collection of markers, namely the BF7 polypeptides listed herein (SEQ ID NO: 1 to SEQ ID NO: 28) by binding a motif that shares common characteristics among the polypeptides, as exemplified by the ability of the monoclonal antibody to react with at least one of the mimotopes listed herein (SEQ ID NO: 29 to SEQ ID NO: 31; FIG. 1). The antibodies encompassed by the present invention include all antibodies, as defined above, that are capable of binding (having specific binding affinity) to BF7 markers, both naturally occurring in humans and synthesized by known chemical or biological techniques, and preferably those polypeptide variants containing the binding motif.

The reactivity of monoclonal antibody BF7 of the present invention is specifically targeted to the BF7 polypeptides (SEQ ID NO: 1 to SEQ ID NO: 31) encoded by the corresponding polynucleotides (SEQ ID NO: 32 to SEQ ID NO: 63) OR to polypeptides (SEQ ID NO: 1 to SEQ ID NO: 31) encoding the BF7 markers, as long as such species harbor the BF7 binding motif facilitating use of the marker in any embodiment of the present invention. The specific novel monoclonal antibody BF7 disclosed herein may also be reactive against proteins or fragments thereof not listed herein that share substantial similarity in antigenic determinants or structural domains (substantially similar epitopes, e.g. mimotopes). Indeed it is well-established and known to those skilled in the art that protein families performing similar cellular functions share functional domains in the form of highly conserved amino acid sequence motifs, which become the “functional” signature of those given proteins and their variants (polymerase, kinase, protease, etc.). Hence, other related target polypeptides may share amino acid motifs or functional domains with BF7 markers listed herein.

A “biological sample(s)” as referred to herein is a quantity of tissue, or body fluid or other material from human patient or normal controls, and comprises tissues and/or biological fluids containing a polypeptide expressed by the patient. Tissue samples include, but are not limited to fresh or frozen normal or diseased tissues (including normal, tumor adjacent tissues), particularly cancer tissues, such as derived from a tumor biopsy cell line (lysate or intact) extracts, including the extracts of the MPAT assay described below, or any other preparation that may be processed for advantageous use in the methods or kits of the invention, and including from different organ sites, different histological types of cancer, and different stages (early, advanced, metastatic), but also tissues from benign and/or inflammatory conditions at a given organ site. A “biological fluid” includes any body fluid from a human patient used for detection of secreted proteins. The term “patient” refers to a human previously diagnosed with disease or an asymptomatic person screened for disease. Examples of patient test samples include, but are not limited to, blood, lymph, serum, plasma, urine, gynecological fluids and smears, bronchio-alveolar lavages, sputum, nipple aspirate fluids, etc. In many instances, such samples are associated with the detection of diseases and conditions at specific organ sites, e.g. bronchio-alveolar lavages for asthma, lung cancer and lung diseases, nipple aspirate fluids for breast cancer, urine sediments after digital rectal examination for prostate cancer, etc. Among body fluids, serum and urine are particularly important, as they represent an informative biological material not requiring invasive procedures.

In preferred embodiments, the biological samples examined are matched normal and tumor tissues derived from the same patient including, normal adjacent tumor samples derived from the same or different cancer patients. Samples may include primary tumor or metastasis, early or late stages of cancer, from stage I to stage IV, as well as benign tumors and inflammatory conditions. For the purpose of the present invention, biological samples referred herein may also include mammalian cell cultures, preferably cancer cell lines, as well as microdissected cell types from normal or disease tissue samples, or from a given subcellular compartment.

### BF7 Polypeptides.

The present invention includes compositions comprising and methods using polypeptides having the amino acid sequences (SEQ ID NO: 1 to SEQ ID NO: 31) that are recognized by monoclonal antibody BF7 of the present invention whereby the detection of said polypeptides, alone, in combination of 2, 3, 4, or more, or collectively, enables the discrimination of patients with colorectal (CRC) cancer from normal controls thus finding application in the early detection of cancer, in the early detection of disease relapse, and in the monitoring of therapy response. As specified above, any combination of 2, 3, 4, 5, 6, 7, 8, 9, 10, 11, 12, 12, 13, 14, 15, 16, 17, 18, 19, 20, 21, 22, 23, 24, 25, 26, 27, 28, 29, 30, 31 proteins is contemplated by the present invention.

The protein identity of BF7 markers recognized by the BF7 monoclonal antibody can be determined by a variety of approaches known in the art, including but not limited to epitope mapping by phage display, human expression library immunoscreening, and proteomics (immunocapture mass spectrometry), as described in Examples 1-2. Epitope mapping by phage display is based on the screening of a library of phages displaying a population of 12-mer peptides on the phage capsid, such population comprising random combinatorial sequences of amino acids that are not naturally-occurring in a living organism. The best 12-mer binders to BF7 monoclonal antibody were selected, as described in detail in Example 1. Insofar this method results in the identification of mimotopes having homology to the BF7 epitope (FIG. 1). BLAST search of the NCBI protein databases (blast.ncbi.nlm.nih.gov) may provide the identity of polypeptides recognized by BF7 Specifically the sequence of the best binding peptide is entered in the BLAST search to retrieve proteins featuring a sequence with highest homology to the queried peptide, as described in detail in Example 2.

BF7 marker polypeptides recognized by BF7 monoclonal antibody may be identified through other methods, such as human expression library immunoscreening. In another embodiment, we screened high-density protein macroarrays containing 27,648 individual E. coli expressed protein clones from a human fetal brain cDNA expression library (Bussow et al., 1998), with the BF7 monoclonal antibody, as described in Example 2. BF7 reactivity with specific proteins expressed in situ from bacteria spotted on the macroarrays was analyzed, scored and the corresponding clone sequences were identified through the RZPD database (https://www.ebi.ac.uk/arrayexpress/files/A-GEOD-15009/A-GEOD-15009.adf.txt).

BF7 marker polypeptides recognized by the BF7 monoclonal antibody and disclosed herein are: Sterile alpha motif domain-containing protein 14 (SAMD14, SEQ ID NO: 1); Nuclear pore complex protein Nup107 (NUP107, SEQ ID NO: 2); Probable rRNA-processing protein EBP2 (EBNA1BP2, SEQ ID NO: 3); Transcriptional activator MN1 (MN1, SEQ ID NO: 4); FH1/FH2 domain-containing protein 1 (FHOD1, SEQ ID NO: 5); Putative IQ motif and ankyrin repeat domain-containing protein LOC642574 (LOC642574, SEQ ID NO: 6); UPF0488 protein C8orf33 (C8orf33, SEQ ID NO: 7); 28S ribosomal protein S11, mitochondrial (MRPS11, SEQ ID NO: 8); Transcription initiation factor TFIID subunit 4 (TAF4, SEQ ID NO: 9); Serrate RNA effector molecule homolog (SRRT, SEQ ID NO: 10); 40S ribosomal protein S13 (RPS13, SEQ ID NO: 11); 60S ribosomal protein L4 (RPL4, SEQ ID NO: 12); Voltage-gated potassium channel subunit beta-3 (KCNAB3, SEQ ID NO: 13); Plakophilin-3 (PKP3, SEQ ID NO: 14); Kelch-like protein 14 (KLHL14, SEQ ID NO: 15); Ras-related protein Rab-27B (RAB27B, SEQ ID NO: 16); 6-phosphogluconolactonase (PGLS, SEQ ID NO: 17); Cyclin L2 (CCNL2, SEQ ID NO: 18); Iron-sulfur protein NUBPL (NUBPL, SEQ ID NO: 19); Protein Bassoon (BSN, SEQ ID NO: 20); Histone H1.2 (HIST1H1C, SEQ ID NO: 21); Ubiquilin 4 (UBQLN4, SEQ ID NO: 22); Cytosolic Fe—S cluster assembly factor (NUBP2, SEQ ID NO: 23); Rho-related GTP-binding protein RhoU (RHOU, SEQ ID NO: 24); Collagen alpha-1 (I) chain (COL1A1, SEQ ID NO: 25); Galectin-3 binding protein (LGALS3BP, SEQ ID NO: 26); Tetraspanin (TSPAN7, SEQ ID NO: 27); Ras-related protein RAB-11B (RAB11B, SEQ ID NO: 28).

The present invention comprises the BF7 amino acid sequences listed (SEQ ID NO: 1 to SEQ ID NO: 31) as well as a population of polypeptides having related or identical sequences to SEQ ID NO: 1 to SEQ ID NO: 31 such as isoforms, fragments, variants and derivatives thereof, and related polypeptide variants sharing homology with the binding motif of the BF7 antibody, as defined by the 12-mer mimotopes (FIG. 1).

The compositions of the BF7 polypeptides include any non-naturally occurring species manufactured from the synthetic polynucleotide sequences claimed herein by conventional and non-conventional mechanisms, such as frameshift, either occasional or programmed, internal initiation, or non Watson-Crick codon-anticodon pairing events at the translation level. These and other mechanisms may lead to the production of hybrid or synthetic BF7 polypeptides, for example carrying amino acid motifs of one reading frame and/or amino acid motifs expressed from another reading frame. Synthetic or hybrid polypeptides may retain substantially the same biological function or activity as the relevant biomarker while partially differing in any degree from the natural amino acid sequence.

Non-naturally occurring variants of the BF7 proteins can readily be generated using recombinant techniques. Such variants include, but are not limited to, deletions, additions, and substitutions in the amino acid sequence of the BF7 protein markers. For example, one class of substitutions is conserved amino acid substitutions. Such substitutions are those that substitute a given amino acid in a BF7 polypeptide by another amino acid of like characteristics. Typically seen as conservative substitutions are the replacements, one for another, among the aliphatic amino acids Ala, Val, Leu, and Ile; interchange of the hydroxyl residues Ser and Thr; exchange of the acidic residues Asp and Glu; substitution between the amide residues Asn and Gln; exchange of the basic residues Lys and Arg; and replacements among the aromatic residues Phe and Tyr. Guidance concerning which amino acid changes are likely to be phenotypically silent is found in Bowie et al., Science 247:1306-1310 (1990). Such amino acid substitutions underly the concept of a BF7 common binding motif and correlate, for example, BF7 mimotopes (FIG. 1) to BF7 polypeptide sequences listed herein.

Included herein are variants of the BF7 marker polypeptide sequences obtained by recombinant or gene synthesis techniques using sequences from the relevant as well as from different expressed proteins, such as mimicking the products of gene fusions naturally occurring in cancer, as long as those fusion constructs preserve the BF7 binding motif. Indeed structural chromosome rearrangements result in the exchange of coding or regulatory DNA sequences between genes. Many such gene fusions are mutation drivers in cancer and have been associated to tumorigenesis (Mertens et al. Nat Rev Cancer 15:371-381, (2015)).

Variants of the BF7 marker polypeptides may also be comprised of non-naturally occurring modifications to the BF7 polypeptides including, but not limited to, acetylation, acylation, ADP-ribosylation, ubiquitination etc.

BF7 marker polypeptides encompassed by the present invention may include fusion to a marker sequence supplied by an expression vector and enabling purification of the polypeptide of the present invention, such as hexa-histidine tag, glutathione-S-transferase, hemagglutinin, luciferase, beta-galactosidase, and the like. The polypeptides may also include polypeptides, in full or in part, modified by any form of post-translational modification, such as phosphorylation, acylation, methylation, ubiquitination, etc., conjugation or covalent linkage to lipids, polysaccharides and the like. These polypeptides further include full-length mature folded proteins, or fragments thereof, either derived by internal initiation, early termination, degradation, or post-translational processing. Non-naturally occurring polypeptide variants of BF7 marker polypeptides may be distinguished from naturally occurring forms by several parameters including characterizing unique sequence content, conjugation with other chemical species, alterations in glycosylation or other chemical signatures including sialylation, any altered structural or chemical composition resulting from expression in non-mammalian expression systems or organisms, altered folding characteristics from non-mammalian expression or processing including measured variances in folding structure caused by separation on a column or other purification or processing techniques.

Variant polypeptides of BF7 marker polypeptides also include isolated antigenic determinants, epitope sequences, or other structural protein domains, produced by different methods known those skilled in the art, including but not limited to: direct peptide synthesis using conventional solid-phase techniques (Merrifield, 1963), direct gene synthesis, in vitro run-off transcription from vectors carrying bacteriophage promoters, high-throughput cell-free translation systems (Sawasaki, 2002), and by recombinant techniques aiming at the expression and purification of recombinant proteins or protein fragments from bacterial, yeast, insect, or mammalian expression vectors that are commercially available and known to those in the art.

Variant polypeptides of BF7 markers can also be purified from cells that express them, purified from cells that have been altered to express them (recombinant), or synthesized using known protein synthesis methods (e.g., Sambrook et al., Molecular Cloning: A Laboratory Manual. 3rd. ed., Cold Spring Harbor Laboratory Press, Cold Spring Harbor, N.Y., (2001)). For example, a natural or synthetic polynucleotide encoding a BF7 marker protein is integrated into an expression vector, the expression vector introduced into a host cell, and the non-naturally occurring BF9 polypeptide variant expressed in the host cell. The polypeptide variant can then be isolated from the cells by an appropriate purification scheme using standard protein purification techniques.

### BF7 Polynucleotides.

The present invention includes polynucleotide species which encode BF7 polypeptides (SEQ ID NO: 1 to SEQ ID NO: 31) that are recognized by the BF7 monoclonal antibody of the present invention and that alone, in combination with at least another, or collectively, enable the discrimination with high probability of patients with CRC cancer from normal controls thus finding application in the early detection of cancer, in the early detection of disease relapse, and in the monitoring of therapy response.

### Bf7 Genes.

Exemplary nucleic acid molecules of the invention consist essentially of, or comprise a nucleotide sequences that encode BF7 marker proteins of the invention, allelic variants thereof, and orthologs or paralogs thereof for example. As used herein, a synthetic polynucleotide bears chemical signatures resulting from defined differences between the synthetic entity and the nucleic acid sequence of the natural polynucleotide. Preferably, the synthetic polynucleotide is free of sequences which naturally flank the nucleic acid (i.e. sequences located at the 5′ and 3′ ends of the nucleic acid) in the genomic DNA of the organism from which the natural polynucleotide is derived. The synthetic polynucleotide typically includes synthetic flanking sequences, particularly contiguous protein-encoding sequences and protein-encoding sequences within the same gene but separated by introns in the genomic sequence, and flanking nucleotide sequences that contain regulatory elements. The primary consideration is that the nucleic acid is distinguishable from the naturally occurring sequence by engineered or manufactured manipulations described herein including recombinant expression, the design and preparation of probes and primers, and other features such as a non-naturally occurring transcript/cDNA molecule, or synthetic polynucleotide produced by recombinant technique, or chemical synthesis.

A synthetic polynucleotide can be comprised of the naturally occurring polynucleotide and fused to other coding or regulatory sequences and still be considered synthetic. Synthetic polynucleotides can include heterologous nucleotide sequences, such as heterologous nucleotide sequences that are fused to a nucleic acid molecule by recombinant techniques. For example, recombinant DNA molecules contained in a vector are considered synthetic. Further examples of synthetic DNA molecules include recombinant DNA molecules maintained in heterologous host cells, or purified (partially or substantially) non-naturally-occurring DNA molecules in solution. Synthetic pre-RNA or RNA molecules include in vivo or in vitro RNA transcripts of synthetic DNA molecules as long as the species is not naturally occurring, but may include species produced by unusual or rare phenomenon. Synthetic nucleic acid molecules further include such variant molecules produced synthetically.

Synthetic polynucleotides encode a mature protein plus additional amino or carboxyl-terminal amino acids, or amino acids interior to the mature protein (when the mature form has more than one peptide chain, for instance). Such sequences may play a role in processing of a protein from precursor to a mature form, facilitate protein trafficking, prolong or shorten protein half-life, or facilitate manipulation of a protein for assay or production, among other things. As generally is the case in situ, additional amino acids may be processed away from the mature protein by cellular enzymes.

Synthetic nucleic acid molecules include, but are not limited to, sequences encoding a BF7 polypeptide variant alone, sequences encoding a mature protein with additional coding sequences (such as a leader or secretory sequence (e.g., a pre-pro or pro-protein sequence), and sequences encoding a mature protein (with or without additional coding sequences) plus additional non-coding sequences (e.g., introns and non-coding 5′ and 3′ sequences such as transcribed but non-translated sequences that play a role in transcription, mRNA processing (including splicing and polyadenylation signals), ribosome binding, and/or stability of mRNA). In addition, synthetic polynucleotides can encode BF7 polypeptide variants that facilitate purification.

Synthetic polynucleotides including cDNA and genomic DNA obtained by cloning or produced by chemical synthetic techniques or by a combination, can be double-stranded or single-stranded. Single-stranded nucleic acid can be the coding strand (sense strand) or the non-coding strand (anti-sense strand).

Synthetic polynucleotides are non-naturally occurring variants made by random or targeted mutagenesis techniques, including those applied to isolated nucleic acid molecules, cells, or organisms. Accordingly, nucleic acid molecule variants can contain nucleotide substitutions, and sequence deletions, inversions, and/or insertions can occur in either or both the coding and non-coding regions, and variations can produce conservative and/or non-conservative amino acid substitutions.

A fragment of a synthetic polynucleotide typically comprises a contiguous nucleotide sequence at least 8, 10, 12, 15, 16, 18, 20, 22, 25, 30, 40, 50, 100, 150, 200, 250, 500 (or any other number in-between) or more nucleotides in length and encodes epitope bearing regions or binding motif of the encoded BF7 polypeptides particularly for separation of the protein from related isoforms or variants as DNA probes and primers.

A probe/primer typically comprises a substantially purified oligonucleotide or oligonucleotide pair. An oligonucleotide typically comprises a nucleotide sequence that hybridizes under stringent conditions to at least about 8, 10, 12, 14, 15, 16, 17, 18, 19, 20, 21, 22, 23, 24, 25, 30, 40, 50 (or any other number in-between) or more contiguous nucleotides.

As used herein, the term “hybridizes under stringent conditions” is intended to describe conditions for hybridization and washing under which nucleotide sequences encoding a protein at least 60-70% homologous to each other typically remain hybridized to each other. The conditions can be such that sequences at least about 60%, at least about 70%, or at least about 80% or more homologous to each other typically remain hybridized to each other. Such stringent conditions are known to those skilled in the art and can be found in, for example, Current Protocols in Molecular Biology, John Wiley & Sons, N.Y. (1989-2006). One example of stringent hybridization conditions is hybridization in 6× sodium chloride/sodium citrate (SSC) at about 45° C., followed by one or more washes in 0.2×SSC, 0.1% SDS at 50-65° C.

### Biomarker Detection in Cancer

Monoclonal antibody BF7 enables the detection of BF7 marker polypeptides, through specific binding of the antibody to said polypeptides carrying a BF7 binding motif, and enables measurement of their expression in biological samples, and patient test samples, particularly detection in protein samples from normal and disease human specimens, where the disease is cancer, and specifically colorectal (CRC) cancer, or from cancer tissue or cell lines, or from patient biological fluids, such as serum and urine, thus correlating expression of BF7 marker proteins with the presence of cancer. Differential expression of BF7 biomarkers in cancer versus normal can be detected via immunodetection of said polypeptides with the BF7 antibody as shown in the following preferred embodiments of the present invention.

### Detection by Matrix Protein Array Technology.

Assessment of differential expression of BF7 marker polypeptides includes immunodetection, and specifically using monoclonal antibody BF7 in a process named the Matrix Protein Array Technology (MPAT) as presented below and described in detail in Example 3.

The MPAT is a multiplex protein array immunoassay that simultaneously analyzes multiple biological samples. In essence, the MPAT is an immunoassay linked to a data acquisition and imaging system, whereby the same matrix of samples is simultaneously interrogated by an antibody. Then, a secondary antibody, preferably linked to a chemiluminescent probe or fluorescent dye, is used to visualize antigen-antibody reaction for each sample, and a scanned image of all reactions is produced with an imaging system, processed and analyzed to yield the simultaneous examination in multiplex format of the relative expression levels of a number of proteins of interest.

The solid support of the matrix protein array is preferably nitrocellulose or glass, yet can be made of a variety of materials that include, but are not limited to: plastic, polystyrene, nylon, teflon, ceramic, fiber optic and semiconductor materials. The solid support of the matrix protein array is composed of different physical areas that can be referred to as wells, compartments, surfaces, and the like, distinctly separated from each other. These physical areas can adopt a variety of surfaces and volumes, and the support can accommodate from 1 or 2 to more than 10,000 compartments, depending on the needs, leading to an extremely versatile tool. Each compartment may contain biological samples from the same type, different types, the same species, different species, the same physiological condition, different physiological conditions or any combination of the above arrayed on the solid support. Each compartment is overlaid with any identifier, preferably an antibody, as selected.

It is understood by those skilled in the art that the device and methodology described herein as MPAT allows all kind of combination of biological samples, number of samples, conditions of samples, size of compartment of the matrix protein arrays, type of identifiers, or any permutation of the above. Furthermore, while in the present invention, the MPAT methodology described below is applied to human biological samples, it is understood to the skilled in the art that the MPAT is widely applicable to protein samples derived from any organism, including animal, bacterium, yeast, fungus, or plant.

In its simplest format, the MPAT is composed of 96 chambers although other formats can be used depending on the number of antibodies to assay and the number of samples to screen. In a given MPAT experiment, the same matrix of protein extracts from different biological samples (e.g. clinical specimens or cancer cell lines as described below) is printed in each chamber, and each chamber is assayed with a distinct individual antibody. Each individual compartment is then overlaid with a distinct antibody and processed for the detection of antigen-antibody complexes. This format allows direct comparison between multiple samples (including normal and diseased samples) under the same conditions, preventing day-to-day experimental variability, as it is often observed in other proteomic studies (Diamandis EP, Analysis of serum proteomic patterns for early cancer diagnosis: drawing attention to potential problems, J Natl Cancer Inst 96:353-356, 2004a; Diamandis EP, Mass Spectrometry as a diagnostic and cancer biomarker discovery tool, Mol Cell Proteomics 3:367-378, 2004b; Ransohoff DF, Rules of evidence for cancer molecular-marker discovery and validation, Nature Rev Cancer 4:309-314, 2004) or DNA microarray experiments (Dudoit S, Gentleman RC, Quackenbusch J, Open source software for the analysis of microarray data, Biotechniques 34: S45-S51, 2003; Gabor Miklos GL and Maleszka R, Microarray reality checks in the context of a complex disease, Nature Biotechnol 22:615-618, 2004).

### Detection by Immunohistochemistry.

Immunohistochemistry (IHC) is a commonly practiced in vitro diagnostic procedure to determine normal vs. disease in a patient tissue biopsy. The patient tissue biopsy is first formalin-fixed and paraffin-embedded, then sectioned at 3-5 micrometer thick and mounted on treated microscope glass slides to enhance tissue adherence. Slides are stained with a relevant antibody against a cellular marker in a procedure described in details in Example 6. Tissue microarrays (TMA) can also be used instead of individual slides to analyze the reactivity of an antibody, or marker expression, in a large number of patient samples to establish marker prevalence in a biological sample of the patient.

Encompassed in this invention is performing IHC analysis with monoclonal antibody BF77 and tissue slides featuring cancer tissues and normal controls, preferably adjacent normal controls (NAT above). Typically an immunostaining procedure comprises the use of anti-mouse IgG biotinylated secondary antibody followed by streptavidin linked to horseradish peroxidase, finally followed by the addition of AEC substrate. Other immunostain procedures are contemplated: for example, protocols based on different labeling and detection systems, such as alkaline-phosphatase, biotin-streptavidine, or fluorophores can also be successfully performed within the scope of the present invention. Furthermore, while most tissues undergo pre-treatment to inactivate endogenous peroxidase, if peroxidase-based staining is used, pre-treatment is not necessary when using fluorescence-based imaging system.

Knowledge of biomarker localization is important in diagnostic applications. Proteins have different localization within the cell depending on their function, including secreted (such as growth factors, hormones, neuropeptides), present on the cell surface (such as glycoproteins, glycolipids and receptors) intracellular (within the cytosol, or in particular sub-cell compartments, such as the nucleus, the Golgi, or the endoplasmic reticulum). BF7 marker polypeptides can be localized to cellular structures via the use of BF7 antibody by a variety of techniques known to those skilled in the art, which can be performed on mammalian cell suspension or adherent cells, and which are described in (Current Protocols in Immunology, Wiley Interscience, John E. Colligan et al.), such as but not limited to, immunohistochemistry, immunfluorescence (IF) using FACScan (FACS), flow cytometry (FC) and indirect IF, but also electron microscopy and other imaging techniques providing localization to subcellular structures.

### Detection by Western Blot.

Encompassed herein is Western blot analysis, as described in details in Example 7, using BF7 antibody to characterize BF7 markers in protein extracts from human cancer cell lines, protein extracts derived from matched or unmatched normal and tumor tissue samples from cancer patients, or from any other biological sample as defined herein above. Protein extracts are separated by gel electrophoresis, transferred to nitrocellulose and probed with BF7 antibody to visualize the corresponding antigen protein bands.

### Detection of BF7 Marker Secretion in Cancer Cell Culture Medium.

In another embodiment of the invention, the BF7 antibody enabled the detection of BF7 markers in serum free cell culture medium (SFCM) of a colorectal cancer cell line (SW116) as biofluid surrogate (prepared as detailed in Example 8), by-ELISA. BF7 markers, or fragments thereof displaying a BF7 binding motif were indeed detected in this medium (FIG. 2). This result demonstrates that BF7 polypeptide markers are secreted in the culture supernatant of the SW116 cancer cell line, suggesting that they may be further secreted in patient biological fluids, including in patient serum.

### BF7 Serum-Based Sandwich ELISA: Assay Development.

A sandwich ELISA assay was developed to detect BF7 markers in patient serum, as described in detail in Example 9, FIG. 2 and its legend. The BF7 assay is a typical sandwich ELISA assay using matched capture and detection monoclonal antibody pair to detect BF7 polypeptides in patient serum. A standard immunoassay protocol involves coating a 96-well plastic tray with capture monoclonal antibody, blocking unoccupied sites, incubating with antigen source (patient serum, other patient biological fluid, or see below), binding captured BF7 polypeptides with biotinylated detecting monoclonal antibody, amplifying detection signal with streptavidin linked horseradish peroxidase (HRP) and revealing the resulting colorimetric reaction with HRP substrate (3,3′,5,5′-Tetramethylbenzidine, TMB) by measuring absorbance (OD) at 450 nm with a microplate reader

Thus the actual assay output is an optical density: the higher the optical density, the more antibody-antigen interaction is detected in the assay, and the higher the levels of BF7 polypeptides in patient serum. To translate the measurement of an optical density, into measurement of marker levels in a patient, a standard calibration curve is used in the art. A standard calibration curve for BF7 is shown in FIG. 2 and described in more detail in Example 9. Briefly, known amount of antigens are measured in the assay thus enabling plotting OD values (on the y axis) against concentrations or antigen levels (on the x axis). Calibration curves thus enable a clinical laboratory running the assay or a software incorporated into the assay to transform raw assay data into biomarker levels in patient serum.

However to further determine whether a patient health status is “diseased”, “normal” or maybe “suspicious”, marker levels in a patient need to be compared to a reference value. Reference values are typically obtained in a population of healthy individuals of same age group and gender than the disease of interest (e.g., male and female of >50 years old in CRC screening). Reference values may vary depending on the clinical application of the assay, such as reference values to detect early stage cancer may differ from reference values to monitor occurrence of disease relapse in a population of patients who has already suffered a primary cancer and is under follow-up, or such as reference values in a population of patients under treatment who are being monitored with respect to therapy efficacy.

The reference value thus establishes the threshold (also known as cutoff) above which the test score is considered “positive” thus defining the person as diseased, or as suspicious needing further procedures, or at risk of carrying the disease etc. Accordingly diagnostic performance is defined by the terms “sensitivity” (proportion of truly diseased subjects in the screened population or test who are identified as positive or “diseased” by the test), and “specificity” (proportion of persons without the disease who have scores below the cutoff on the test) with respect to a given reference value or cutoff, preferably expressed with respect to a given clinical application. All such parameters, but not limited to, come into play when a diagnostic assay measures biomarkers in a patient.

To ensure that the BF7 immunoassay was suitable for measuring clinical serum samples, titration curve and reproducibility of the assay were established (FIG. 2). The inter-assay precision was determined for two patient serum samples (a pool of serum samples from patients known to display high levels of markers, and a pool of serum samples from patients known to display low levels of markers) over 4 days and coefficient of variation (% CV) was <15%, a % CV within the accepted limits.

### BF7 Diagnostic Performance in CRC Early Detection

The diagnostic performance of the BF7 assay was first tested on Clinical Sample Set 1 comprising 384 serum samples from 275 CRC patients, 40 patients presenting with polyps (21 with advanced adenoma (AA) and 19 with polyps of unknown features) and 69 NL controls. The composition of the clinical sample sets used herein is described in detail in Example 12. The clinical characteristics of clinical sample set 1 are summarized in Table 1, with breakdown of number, type, stage, age, gender, whenever available. As indicated 50% of the CRC cases were early stage thus enabling analysis of diagnostic performance in early detection.

BF7 marker levels were measured with the BF7 ELISA assay in duplicate in patient serum samples diluted 1:10 in assay diluent, and BF7 marker concentrations were derived from the BF7 biomarker standard curve as described using 4-parameter logistic method (Example 9, FIG. 2 and legend).

The scatter plot representation of all BF7 measurements in this clinical sample set illustrates BF7 marker distribution in each group, namely in all CRC stages, early and late CRC stage, advanced adenoma and normal controls (CRC, CRC-E, CRC-L, AA, NL; FIG. 3A). Statistical analysis (Example 10) of two or multiple group comparisons was carried out based on the median value of the BF7 biomarker concentration (μgE/ml) in each group to determine whether differences between groups were statistically significant.

Striking differences in the median level of BF7 biomarkers in the various groups are observed (FIG. 3A). Specifically, statistically significant differences (p<0.05) were found in the comparisons between cancer, early or late stage, and normal controls (CRC, CRC-E, CRC-L versus NL), as well as between early and late stage of CRC (CRC-E versus CRC-L).

ROC curve analysis was performed to establish biomarker diagnostic performance (FIG. 3B-D, Table 2). The BF7 assay enabled discrimination of CRC (all stages) versus NL with a sensitivity (SE) of 64.3% at a specificity (SP) of 90% (Table 2) with an area under the curve (AUC) of 0.843 (95% CI 0.801-0.886; FIG. 3B). When early and late stage CRC were considered, BF7 discriminated early CRC (stage I and II) versus NL and late CRC (stage III and IV) versus NL with AUC of 0.797 and 0.893, respectively (p<0.0001; FIGS. 3C and 3D). These data indicated excellent discriminatory power of the BF7 assay not only in the CRC versus NL comparison, but also in early stage CRC and late stage CRC versus NL.

When further analyzing by stage, sensitivity of 52.9%, 55.3%, 66.2%, and 85.7% were obtained at 90% SP in the comparison stage I-IV versus NL, respectively, as summarized in Table 2, indicating increased sensitivity of cancer detection with advanced disease.

Albeit not recommended for screening, CEA is the traditional biomarker used in CRC management. As a comparison we measured CEA levels, alongside BF7, with a commercial anti-CEA antibody pair, and determined CEA diagnostic performance in this sample set. (Table 2). At 90% SP, CEA detected 57.5% of CRC cases (all stages) versus 64.3% for BF7. Except for stage IV where CEA performed slightly better than BF7 with 92.8% SE versus 85.7%, in all other stages, BF7 detected CRC cases with superior sensitivity than CEA. In stage I, in particular, CEA displayed very limited sensitivity (31% SE versus 52.9% for BF7). CEA sensitivity obtained in this clinical sample set (Table 2) is in agreement with the literature (Duffy, 2006).

Combining the BF7 and CEA assays enabled to capture additional CRC cases without impacting the overall specificity, thus yielding successful complementation. Indeed the BF7/CEA combined assay reached a maximum diagnostic performance in this sample set of 74.8% SE at 90% SP for all CRC stages (Table 2). When stages were considered, the combination of the two assays reached over 60% in stage I, 69.4% in stage II, 74.3% in stage III (Table 2). In both cases, the diagnostic performance of the BF7 and CEA assay increases over the four stages of disease, leading to a 96.4% SE in stage IV for the combined panel (versus 85.7 and 92.8 for BF7 and CEA assay alone).

The major result form this study is the ability of the BF7 as a single assay to detect CRC and early stage CRC in patient serum with significant sensitivity at 90% SP, and to reach 74.8% SE at 90% SP in a combination with CEA, indicating the clinical utility of BF7 assay as a test for CRC screening and diagnosis.

The relationship between CRC, polyps and NL was also analyzed. The polyp group comprised 21 advanced adenoma (AA) and 19 polyp cases for which size and other histopathological features were not available. The term advanced adenoma include: adenoma with high grade dysplasia, of any size; adenoma with villous growth pattern (>25%) any size; adenoma>1 cm in size, and serrated lesions of >1 cm in size (Bond, 2000). It is current practice to remove advanced adenoma, at colonoscopy as preneoplastic lesions which are at increased risk of developing into CRC (Fleming, 2012; Zauber, 2012). Therefore the sensitivity of the assay in detecting the AA group was calculated. With the BF7 assay alone, AA was detected with 47.6% SE at 90% SP, while with the combined BF7/CEA assay the AA group was detected with 52.4% SE (Table 2).

Current screening methods have either poor or limited sensitivity towards advanced adenoma: 20-40% at 90% (FIT), 21% SE at 81% SP (Epi-pro Colon), 42.4% SE at 89.8% SP (Cologuard). The combined BF7/CEA assay of the present invention yields 52.4% SE at 90% SP in this clinical sample set, demonstrating a better performance than other current screening methods. Overall the data presented here provides strong evidence for BF7 as an assay for CRC detection and screening either alone or in combination with CEA.

### Use of the BF7 Assay in Monitoring.

The data presented herein demonstrated the ability of the BF7 assay to detect CRC, from advanced adenoma to stage I, stage IV cancer and beyond with increasing sensitivity. The BF7 assay was then applied to monitoring CRC recurrence. BF7 levels were assayed in serum samples collected from patients who were in follow-up after primary CRC resection and treatment, presented with recurrent disease, and had annotated CEA levels measured at hospital visit (Advia Centaur, Siemens). Thus the performance of the BF7 assay could be compared to that of CEA at its standard cutoff values (CEA: 4 ng/ml; Stieber, 2015).

BF7 and CEA marker levels were measured by ELISA assay in a cross-sectional study utilizing 397 serum samples taken from patients in surveillance (Clinical Sample Set 2; Example 12). A cutoff value of 17 μgE/ml was considered for BF7 yielding 82% SP in a CRC versus NL comparison, representing acceptable specificity values in this clinical setting. It was found that the BF7 assay alone yielded 64.6% SE versus 31% SE for CEA, and that the two markers combined reached 78% SE in detecting relapse (FIG. 4).

These data show that: i) the BF7 serum-based assay can detect patients with recurrent and metastatic disease, ii) the BF7 markers recognized by the BF7 monoclonal antibody can serve as early indicators of disease relapse, whether alone, in combination with at least another biomarker, or in combination with other known tumor markers (see below), and iii) that the BF7 assay is more sensitive than the CEA assay in detecting CRC recurrence in the same sample population, indicating that the combined BF7 assay may be a superior alternative to the traditional serum marker in monitoring CRC recurrence.

### Longitudinal Study.

The data described above show that the BF7 assay sensitivity correlates with disease and significantly detects CRC recurrences in a cross-sectional study, complementing and outperforming CEA. To demonstrate early detection of disease recurrence, BF7 biomarkers were evaluated in a set of serial samples collected from patients treated and followed up after a primary CRC and retrospectively obtained (Clinical Sample Set 3; Example 12). Patient clinicopathological features are summarized in Table 3. BF7 markers were measured in duplicate and in a blind fashion. Then, upon sample unblinding BF7 marker levels were matched with well annotated clinical information and analyzed (FIG. 5).

Comparing BF7 and CEA in detecting recurrence. Clinicopathological characteristics of the patient population used in this study are summarized in Table 3. The serum of 91 total patients were obtained retrospectively and tested in ELISA format for BF7 and CEA levels. Since the samples were obtained retrospectively, the frequency of sample collection and the range of follow-up time varied for each patient. For the purposes of this analysis we have separated the patients into three cohorts depending on the number of samples available per patient. Cohort A (n=10) consisted of patients with only two samples, before and at or after recurrence. Cohort B (n=11) consisted of patients with three or more samples where sample collection ended within a month of recurrence diagnosis. Cohort C (n=70) had patients with three or more samples where the sample collection extended to months after recurrence.

Patient sample sets were analyzed for two characteristics with respect to BF7 and CEA serum levels: the ability of the serum markers BF7 and CEA to detect CRC recurrence both alone and in combination, and whether BF7 could show a trend in rising marker level before recurrence was clinically confirmed.

BF7 and CEA were considered to be in positive concordance with the reported patient clinical data if either was above its respective cutoff (COF; >17.1 μgE/ml and 5 ng/ml; respectively) in the sample collected at the nearest time point to diagnosis of recurrence (including at or slightly after recurrence). A marker was considered to be in negative concordance if it was below COF at recurrence, or only went above COF in a sample point past the one nearest recurrence.

Cohort A had a total of 10 patients with only 2 samples, one before and one at or after recurrence. Samples ranged from 3 to 43 months pre-recurrence (average: 13.7) and 1 to 7 months after recurrence (average: 1.2). Of the 10 patients, 8 showed elevated BF7 biomarker levels (>17.1 μgE/ml) at or 1-7 months after recurrence, and 2 showed a decline to under the COF after recurrence. By comparison, CEA exhibited an at recurrence or post-recurrence rise above the COF (5 ng/ml, Locker 2006) in 6 patients, while 4 were under the COF or below the limit of detection. In three patients, BF7 was elevated while CEA remained undetectable or under the COF. In one different patient, the trend was reversed and CEA levels showed an increase while BF7 level slightly decreased albeit still above COF (see below). There was one patient in which neither BF7 nor CEA were above COF at recurrence. However in this case it should be noted that the interval between collection time points was about 4 years and the post recurrence point was 7 months after diagnosis, with no information on any possible treatment that the patient might have incurred during the 7 months after diagnosis of recurrence.

Cohort B had 11 patients who had 3 or more samples available and in which the collection ended within a month of diagnosis of recurrence. Samples ranged from 7-38 months pre-recurrence (average: 16.5) to the time of recurrence. 10 of the 11 showed an elevated value of BF7 at point of recurrence and 1 remained below COF. On the other hand, CEA was shown to be above COF at recurrence for 5 of the 11 patients, and was either below COF or below the limit of detection for the remaining 6. It should be noted that of the 10 patients with elevated BF7, 5 patients also had elevated CEA and 5 patients had CEA below COF.

Cohort C had 70 patients who had 3 or more samples available to test and had samples from several months before and after recurrence, with a total number of samples of 322. Samples ranged from 1-54 months before recurrence (average: 18) and 1-22 months after recurrence (average: 5.7). In 50 of those patients, BF7 was found to be elevated either at time of diagnosis of recurrence, or at the first time point immediately after recurrence (71.4% SE), while 20 patients showed poor correlation between marker level and diagnosis of recurrent malignancy. With respect to CEA, 32 patients showed good correlation, while 38 patients showed poor correlation, where CEA was either below COF or below limit of detection. Of the 50 patients where BF7 was elevated at time of recurrence, 25 also had CEA above COF, and 25 patients had CEA below COF. In 6 of 70 patients only (8.6%), CEA was elevated at recurrence while BF7 was not. In 11 patients, neither marker was found above COF at recurrence.

Taken the results of the 3 cohorts together, BF7 showed an overall positive concordance with diagnosis of recurrence (elevated or >COF) in 68 out of 91 patients (74.7% SE). On the other hand, CEA had a positive concordance of 43 out of 91 patients (47.3% SE), a sensitivity in line with what has been previously reported (Duffy, 2001; Brunner, 2006; Fakih, 2006). When combined, the two serum markers yielded a positive concordance of 76 out of 91 patients (83.5% SE). These data indicate that while BF7 alone provides superior sensitivity in detecting recurrence events over CEA, the two markers complement each other, and the combined panel reaches an even higher overall sensitivity (Table 4).

We also analyzed the results with respect to the site of metastasis as presented in Table 4. BF7 performs better than CEA in every category. Seven patients presented with local recurrence, and the sensitivity of BF7 and CEA at the time point nearest to recurrence was 71% and 43% respectively. Of special note is the higher sensitivity of BF7 over CEA when metastasis is in the lung (56% versus 22% SE), in the liver and some other site (100% versus 56%), and in other distal sites, such as peritoneum, lymph nodes, brain, kidney, skeleton, ovary (85 versus 45% SE).

Correlation between serum marker level and disease progression. The performance of the two serum markers in tracking disease progression in patient samples over time was assessed by analyzing marker elevation from pre-recurrence samples to the time of recurrence. This also enabled us to see how BF7 performed in the months before clinical diagnosis of recurrence to evaluate possible lead time.

In Cohort A (n=10), there were only two collection time points per patient. In the 8 patients mentioned above (where BF7 was elevated at time of recurrence), marker increased, at or after relapse, with respect to the previous time point in 7 patients, in agreement with the hypothesis that an increase in marker level signals disease progression. Average interval between samples was 9.4 months. Interestingly in the remaining patient, the biomarker went from 72 μgE/ml to 35 μgE/ml at recurrence in a 23 month time span, still well above COF. As to CEA, an overall increase at recurrence was observed in 6 out of 10 patients, with an average interval of 11 months between the two collection time points. Cohort A is illustrated by two exemplary patients in FIG. 5A.

Lead time for recurrence was estimated based on samples at least one month after surgery in which marker levels were above COF. In cohort A and based on sample availability, 6 patients showed a lead time with BF7 ranging from 3-43 months with an average of 16.2 months. In this cohort we found that CEA did not yield any lead time because all samples were below COF.

In Cohort B (n=11), each patient had three time points or more, which ended in the month of clinical diagnosis of recurrence. In 7 out of 11 patients, BF7 level increased at recurrence compared to the previous time point, with an average interval between pre and post-recurrence of 9.1 months. In 3 of the remaining patients with average interval of 16.3 months, BF7 was elevated in the months before recurrence diagnosis but decreased at the point closest to recurrence, albeit still above COF. In 1 of those 3 that decrease may be due to intervening surgery. A marker increase between the time point prior to recurrence, and at recurrence was also observed with CEA in 5 out of 11 patients, with an average interval of 10 months. Cohort B is illustrated by two exemplary patients in FIG. 5A. In cohort B and based on sample availability, 6 patients showed a lead time with BF7 ranging from 3-38 months, with an average of 15.3 months. As to CEA, in this cohort, only one sample qualified for lead time calculation, and lead time was 2 months.

Cohort C (n=70) had 3 or more time points extending beyond the diagnosis of recurrence. In 38 out of the 50 patients where BF7 was above COF at recurrence, the marker level rose from the pre-recurrence time point to either at or near recurrence. Average interval between pre and post-recurrence samples was 12.4 months. In the other 12 patients, the marker was elevated before recurrence and decreased at recurrence, although still above COF. With respect to CEA, in 31 out of the 32 patients where CEA was above COF at recurrence, the marker rose in concentration from the pre-recurrence time points to either at or near recurrence, with an average interval of 12.6 months. Cohort C is illustrated by four exemplary patients in FIG. 5B.

Cohort C is particularly suited to investigate lead time due to the large population size and number of sample points. In cohort C and based on sample availability, 47 patients showed a lead time with BF7 ranging from 2-52 months with an average of 19.1 months. Combining the lead times of similar patients from Cohorts A, B and C (n=60), the maximum lead time found was 52 months with an average of 18.4 months before recurrence. In this cohort, CEA lead time range was from 1 to 26 months, with an average lead time of 8.75 months.

Of special interest to clinicians is the ability to gauge the need for adjuvant treatment, especially in the metastatic setting when surgical resection is not an option. A few patients in this study had serum samples that were collected shortly after adjuvant treatment, with follow-up time points between 1 day and 3 months, thus enabling us to evaluate whether BF7 might be useful in monitoring therapy response. Patient 1257, for example, had post surgery adjuvant treatment the same day as when the first sample in the set was collected. When the following sample was collected 9 days later, MIL-774 level decreased from 78.3 μgE/ml to 17.3 μgE/ml. In contrast, CEA in the same interval rose from below the level of detection to 2.1 ng/ml, albeit still below the COF. In patient 36792, treatment started 1 day before the first sample time point. In the second sample point 5 days later, BF7 had dropped from 26.83 μgE/ml to 12.2 μgE/ml. In patient 1785, neoadjuvant radiation and chemotherapy started the same day as the first sample was collected, and 3 months later, BF7 had dropped from above 253.2 μgE/ml to 12.76 μgE/ml. Surgical resection occurred 1 day after, and 2 months later the marker had dropped further to 9.1 μgE/ml. Patient 2107, in contrast, exhibited a pattern showing when treatment is ineffective. Adjuvant treatment started 1 day before the 2nd sample in the set was drawn, yet the concentration of BF7 rose from 31.6 μgE/ml to 54.6 μgE/ml. CEA in both time points remained below the limit of detection. All four patients are shown in FIG. 5C.

This study shows that the BF7 serum marker complements CEA, while yielding greater sensitivity and lead time than CEA alone. Combining both markers provides a better indicator of disease relapse. Therefore these data strongly support the notion that the BF7 assay has clinical application in monitoring changes in patient disease status, either alone or as adjunct to CEA.

### Immunodetection of Secreted BF7 Markers in Urine.

In a preferred embodiment of the present invention, we further detect the presence of BF7 marker polypeptides, fragments thereof, or BF7 binding motif in the urine of cancer patients versus normal controls, using BF7 monoclonal antibody and the MPAT, as described in detail in Example 13.

Briefly, urine samples were centrifuged to remove debris, and equal volumes were spotted, in a double-blind experiment, on the MPAT membrane either “as is” or upon dilution in Tris-Triton buffer. Because of its low protein abundance, we reasoned that attempting to concentrate urine proteins would actually lead to loss of potential biomarkers. As indicated in FIG. 6 legend, a variety of cancer patients was included in the experiment in a 47 sample set (Table 5). Considering the likely possibility of prostate cells shedding into urine, we included samples from prostate cancer patients, representing stages II and III (n=13). Samples from early stage colon cancer patients (n=10; stage I and II), from patients with benign colon disease (n=4) and inflammatory conditions of the colon (n=5) were included as well as some pancreatic cancer cases (n+5) and normal controls (n=10).

As illustrated in FIG. 6, while only background reactivity is observed in normal individuals, BF7 monoclonal antibody clearly reacts with urine proteins in all cancer groups: in colon, pancreatic and prostate cancer. Some reactivity is observed in the colon inflammation cases, while no reactivity at all is observed in the colon benign cases. Specifically, in this experiment and clinical sample set, BF7 antibody detects biomarkers or a fragments thereof carrying the BF7 binding motif in the urine specimens of 50-70% of colon cancer cases, depending on the sample conditions used (FIG. 6, compare reactivity of samples “as is” versus samples diluted 1:2 or 1:10 in Tris-Triton buffer). Lower reactivity is observed in the urine specimens of colon inflammation cases. It should be noted that all colon cases in this experiment are stage I and II. This result emphasizes the ability of BF7 to detect early stage cancer.

With respect to the other cancers, BF7 assay detects 61.5% of prostate cancer patients (mostly in early stage disease) when urine samples are diluted 1:2 in Tris-Triton buffer (FIG. 6, row D), and of 3 out of 5 pancreatic cancer patients (nearly all early stage) in the same sample conditions (FIG. 6, lanes A and B; 1:2). In conclusion, in this experiment and clinical sample set, BF7 reactivity is observed in the urine samples of colon, pancreatic and prostate cancer patients, suggesting that polypeptides or fragments thereof bearing BF7 binding motif are secreted in urine.

Pancreatic cancer, while only representing 6% of estimated new cancer cases in 2012, it is also responsible for 11% of cancer deaths (Siegel R et al., Cancer statistics, 2021, CA Cancer J Clin 62:10-29, 2012). In its early stage, pancreatic cancer is a relatively symptomless disease; hence patients generally present at advanced stage, and only 10-15% of them are surgical candidates, with small resectable cancers (ACS, 2012). For all stages combined, the 1-year relative survival rate is 24%, and the 5-year rate is about 4% (ACS, 2012). Pancreatic cancer has the shortest life expectancy of all malignancies when discovered, with a median survival rate of ˜18 months (ACS, 2012). Furthermore, serum biomarker CA-19, which is elevated in the advanced stage of several malignancies, particularly gastrointestinal cancers, is more clinically useful as prognostic and treatment response marker rather than early detection marker (Goggins, 2005; Duffy, 2010). Hence there is a great need for pancreatic cancer biomarkers, whether based on tissue or biological fluid, with particularly emphasis in the early detection of this malignancy.

Limitations of PSA as early detection marker for prostate cancer have been mentioned above, suggesting the need of additional biomarkers as adjunct to current screening methods, with particular emphasis in urine-based in vitro diagnostics.

### EXAMPLES

The following abbreviations are used throughout. hr: hour; min: minutes; see: seconds; rpm: rotation per minute; RT: room temperature; ON: overnight; mAb: monoclonal antibody.

### Example 1: Epitope Analysis by Phage Display

As part of a characterization of the BF7 mAb, a phage display approach was employed using the New England Biolabs PhD-12 Phage Display Library Kit according to the manufacturer's instruction manual. A brief description of the protocol follows.

The phage library has a titer of 1013 pfu/ml. Ten microliters of the library are incubated in TBST (50 mM Tris-HCl pH 7.5, 150 mM NaCl, 0.1% Tween 20) with the mAb BF7 for 1 hr at RT. The mAb is immobilized on a plastic surface, such as an ELISA 96 well plate, via a rabbit anti-mouse IgG (see below). This represents a ˜1011 pfu input, i.e. a ˜ 100 fold representation of a library with a complexity of 109 individual clones, each harboring five copies of a 12-mer peptide embedded in the phage capsid. After incubation and washing of unbound phages in TBST, bound phages are eluted in 0.2 M glycine-HCl pH 2.2, 1 mg/ml BSA, then neutralized in 1 M Tris-HCl pH 9.1 and finally amplified in the appropriate bacterial strain (ER2731) by growing for 4-5 hrs at 37° C. with vigorous shaking. Phage is collected upon removal of bacterial cells by centrifugation, and precipitated by 20% PEG in 2.5 M NaCl at 4° C. ON. Phage is titered in order to carry out a second and third panning with an input titer equivalent to that of the first round. Note that stringency increases from the first to the third panning, by increasing Tween 20 concentration (0.1% to 0.5%) and decreasing incubation time (1 hr to 30 min).

To decrease non-specific binding of the phage library, the three panning steps are first preceded by a pre-panning (prior to incubation of phage library with the relevant mAb), whereby the phage library is incubated with non-specific or pre-immune mouse IgG immobilized to 96 well plates via a rabbit anti-mouse IgG. This pre-panning incubation step eliminates all phages in the library that would non-specifically bind to plastic, rabbit anti-mouse IgG and mouse IgG. Rabbit anti-mouse IgG plate coating is used to concentrate the relevant mAb (or the mouse IgG in the pre-panning step), which is in the form of hybridoma culture supernatant rather than in the form of purified mAb; such coating is also used to enhance mAb binding to the plate. Pre-panning is carried out ON at 4° C. or 1 hr at 37° C.

Upon the three cycles of panning and amplification, an enriched population of phage is collected; this enriched population harbors, within the capsid amino acid sequence, those 12-mer peptides that are specifically bound by mAb BF7, and thus represent, at least in part, the epitope of BF7 marker polypeptides. In fact they are preferably called mimotopes to indicate that they comprise sequences displaying homology to the actual epitope yet not the exact actual sequence.

After the third round of panning, resulting phages are titered (yet not amplified), and about 20 individual phage plaques are picked, amplified in a small volume of bacterial culture, and phage DNA is prepared upon a simple phenol chloroform extraction followed by ethanol precipitation. Phage DNAs are sequenced and the 12-mer peptide amino acid sequence is determined for each clone.

Non-redundant individual phages (i.e. with different 12-mer sequences) are amplified in medium sized cultures and titered in order to be further assayed by direct ELISA in the presence of mAb BF7. This ELISA assay will identify the clone(s) harboring the best 12-mer binders, as illustrated in FIG. 1. Comparison of the best binder sequences in turn provides insight into mAb BF7 epitope.

Briefly, ELISA plates are first coated with the mAb of the present invention (10-100 μg/ml, diluted when necessary in 0.1 M NaHCO₃pH 8.6) by ON incubation at 4° C., or 1 hr at 37° C. Each step is followed by 10 TBST washes (0.5% Tween 20). Plate wells are then blocked with blocking solution (see above) for 1-2 hr at 4° C. Serial four-fold dilutions of phage to be tested (from 10¹² to 2×10⁵ virions per well) are prepared in a separate pre-blocked 96 well plate in TBST (0.5% Tween 20), and added to mAb BF7 coated wells, and to the uncoated (but blocked) wells used as negative controls, for 1-2 hr incubation at RT with agitation. Phage-mAb complexes are detected by colorimetric reaction at 450 nm involving a 2-step process: i) 1 hr incubation at RT with horse radish peroxidase (HRP) conjugated anti-M13 antibody according to manufacturer's instructions (GE Healthcare), followed by ii) addition of TMB (HRP substrate) until a blue color develops. Signal intensities are compared to the “no mAb” control, and the reaction stopped by H₂SO₄.

### Example 2: Determination of BF7 Marker Polypeptide Identity

In the case of BF7, the sequences of the 12-mer peptides, selected by the phage display approach, that have shown to bind with highest affinity to BF7 monoclonal antibody (e.g. best binders, as described in Example 1 and listed in FIG. 1 and herein) are used to query NCBI protein databases (Non-redundant protein sequences (nr); Reference proteins (refseq_proteins); UniprotKB/swissprot (swissprot)) by protein BLAST (blast.ncbi.nlm.nih.gov). The 12-mer peptide sequences are referred to as mimotopes to indicate that they comprise amino acids displaying homology to the actual BF7 epitope. Therefore the BLAST search yields proteins featuring an amino acid sequence with high homology to the BF7 mimotopes, thus providing in turn the identity of the BF7 polypeptides recognized by BF7 monoclonal antibody.

In addition to phage display, other approaches were used to characterize the monoclonal antibody of the present invention, e.g. human expression library immunoscreening, and proteomics (immunocapture mass spectrometry) were used to identify the BF7 biomarker polypeptides recognized by the BF7 monoclonal antibody. High-density protein macroarrays containing 27,648 individual E. coli expressed protein clones from a human fetal brain cDNA expression library were screened with the BF7 monoclonal antibody (Bussow et al., 1998). Briefly, macroarrays were washed twice in TBSTT (TBS, 0.05% Tween 20, 0.5% Triton X-100) to lyse bacteria and four times in TBS. After blocking with 3% non-fat dry milk in TBST (TBS, 0.05% Tween 20) for 2 hr at RT, macroarrays were incubated overnight with BF7 hybridoma supernatant (diluted 1:5 or undiluted) either 2 hr at RT or ON at 4° C. Macroarrays were washed three times with TBST and exposed for 1 hr t RT to anti-mouse IgG infrared 800 (Li-Cor) as per manufacturer recommendation. After four washes in TBST and two in TBS, signals were detected by scanning with Odyssey imager at the 700/800 nm channels. Macroarrays were analyzed using scoring templates from manufacturer to locate coordinates of duplicate spots, and then referring a particular clone to the RZPD database (https://www.ebi.ac.uk/arrayexpress/files/A-GEOD-15009/A-GEOD-15009.adf.txt).

In another embodiment BF7 mAb was used to to pull down the corresponding antigens from SW1116 protein extracts and SFCM by immunoprecipitation. Captured proteins were separated by SDS-PAGE and Coomassie blue stained. Bands were cut off from the gel and subjected to in-gel trypsin digestion followed by MS analysis. Following Scaffold protein identification software analysis, proteins were selected from MS data hits, based on MS signal intensity, indicating protein abundance, and percentage of peptide coverage, resulting in candidate biomarkers whose sequences shared homologies with mimotopes evidenced by phage display.

### Example 3: Matrix Protein Array Screening Technology

The matrix protein array technology (MPAT) is a multiplex protein array immunoassay developed by the Applicant for the simultaneous analysis of multiple biological samples, under the same conditions. The MPAT has been used for the immunodetection of protein marker/mAb of the present invention in a variety of protein samples, as detailed in the examples below.

The solid support of the matrix protein array may be composed of a different number of chambers or compartments of different sizes, depending on the scope of the investigation. In its simplest format, the MPAT is composed of 96 chambers. Other formats can be used, depending on the number of antibodies to assay, and the number of samples to screen. Biological samples are spotted or printed (see below) in a matrix arrangement within each compartment on a nitrocellulose membrane. The same matrix of clinical samples, including normal and diseased, or the same matrix of protein extracts from different cancer cell lines is printed in each chamber. Each individual compartment is then overlayed with a distinct antibody (polyclonal, monoclonal, Fab fragment, monospecific, single chain, affibodies, or any other recombinant version of conventional or combinatorial antibodies), and processed for the detection of antigen-antibody complexes.

Protein sample analysis. For the purpose of the invention, protein samples analyzed by MPAT may derive from fresh and frozen tissues, whether normal or disease, including from patients with cancer, benign or inflammatory conditions, and normal controls. Protein samples may derive from cell cultures, cancer cell lines, and cancer cell supernatants, and even from microdissected cell types or from a given subcellular compartment. Protein samples may also derive from patient sera or any other patient biological fluid, and prepared as described in the Examples below.

Printing of total protein extracts. Individual protein sample extracts can either be deposited and spotted manually or printed with a robotic system (Genomic Solutions Flexys, PBA Robotics, UK). Routinely equal protein amounts (250 nl of a 1 mg/ml stock solution of cancer cell line protein extract) of each sample are printed in a matrix format on the MPAT membrane, in duplicate or triplicate whenever deemed appropriate. The membrane is then incubated for 30 min in 2% H₂O₂ (hydrogen peroxide) solution to inhibit endogenous peroxidase present in the clinical samples, rinsed twice in Tris-saline buffer (TNE: 10 Tris-HCl pH 7.5, 50 mM NaCl, 2.5 mM EDTA) and then blocked for 30 min with a solution of 1% non-fat dry milk in Tris-saline buffer containing 0.1% (w/v) Tween 20 (TNET).

Antibodies. Subsequently, each chamber or each sample matrix is overlaid with a given primary antibody. Routinely antibodies are diluted appropriately in blocking solution, followed by 1 hr incubation at RT with constant shaking. Blocking solution is TNET containing 1% non-fat dry milk or equivalent blocking solutions.

Detection of antigen-antibody complexes. The membrane is washed 5 times for 5 min each in TNET, then incubated for 1 hr with secondary antibodies conjugated with horseradish peroxidase (Roche) diluted 1:10,000 in blocking solution. The membrane is then further washed 5 times as described previously. The antigen-antibody-anti-antibody complex reactivity is measured by chemiluminescence, using the SuperSignal West Dura Extended Duration Substrate (Pierce). The image is captured using a CCD-camera (charge-coupled device; UVP model Biochemi, CCD camera grade 0, with dark room designed for chemiluminescence, fluorescence and visible).

Alternatively, instead of a chemiluminescent-based detection and a CCD-camera based image acquisition system, a fluorescent-based system can be used, incorporating for example the use of the Li-cor Odyssey infrared imaging acquisition system. The MPAT protocol is then modified accordingly. Peroxidase inhibition is not necessary. The membrane is rinsed twice in Tris-saline buffer, and then blocked for 30 min in Odyssey blocking solution (Li-Cor). Primary antibody is appropriately diluted in Odyssey blocking solution, followed by 1 hr incubation at RT. The membrane is washed 5 times for 5 min each in TNET, then incubated for 1 hr with secondary antibodies labeled with a fluoresecent dye (IgG-IRDye 800CW) diluted 1:10,000 in Odyssey blocking solution. The membrane is then further washed 5 times as described previously. The antigen-antibody-anti-antibody complex is measured by direct infrared fluorescence detection. The intensity of each complex is captured as an image by scanning the membrane with Odyssey infrared imaging system in the 800 nm channel at 84 μm resolution. Protocols based on different labeling and detection systems, such as alkaline-phosphatase, biotin-streptavidine, and fluorophores as described can also be successfully performed within the scope of the present invention.

The following internal controls can be routinely provided: i) the same matrix of samples is overlaid with buffer rather than with primary antibody, followed by the secondary antibody, thus revealing the background of the secondary antibody (no antibody control); and ii) the same matrix of samples is overlaid with pre-immune serum, or non-secreting hybridoma or dilution buffer, followed by secondary antibody, thus revealing the nonspecific binding of mouse immunoglobulins.

### Example 4: Immunodetection of BF7 Marker Polypeptides by Matrix Protein Array in Patient Tissues

Clinical samples. Frozen human tissue biopsies with annotated pathology report were acquired from the Cooperative Human Tissue Network (CHTN). All specimens are tissue samples collected prior to any treatment. Specimens are provided with corresponding pathology report and well-annotated clinical information (disease condition, cancer histological type, clinical history, stage, age, gender, race; Jewell, 2002). Clinical samples are stored in −70° C. freezers, and immediately prior to assay, samples are aliquoted in ice to avoid multiple freeze-thaw cycles, and the original tube is maintained at −70° C. The collection amounts to ˜1,500 cancer tissues covering all major malignancies, comprising colorectal cancer, lung, pancreatic and prostate cancers, melanoma, renal carcinoma, and gynecological cancers, including breast, ovarian, uterine, and cervical cancers.

Protein extraction from frozen tissues. Fresh or frozen tissue of human origin for the purpose of this invention, is cut off in small pieces, grounded, homogenized in a 50 mM Tris-HCl pH 7.5, 2 mM EDTA, 100 mM NaCl, 1% NP40, and 1 mM vanadate solution containing the following protease inhibitors: PMSF, aprotinin, leupeptin at 1, 2 and 4 mM respectively. The homogenate is kept on ice for 20 min and centrifuged at 14,000 rpm for 15 min. Supernatant is transferred to a new container and the tissue pellet is resuspended, and again kept on ice for 20 min and centrifuged as indicated above. Supernatant is removed and added to the first one. Protein concentration is determined according to standard conditions as known to those skilled in the art. Protein solution is stored in a −80° C. freezer until further usage.

MPAT. Protein extracts from frozen tissues are spotted on the MPAT and processed as described in Example 3 using mAb BF7.

Data Analysis. MPAT spots are quantified by the ScanAlyze2 program (Eisen, 2002) which enables robust and high-throughput spot finding on the scanned image produced by the Li-Cor Odyssey infrared imaging system. Quantification of the MPAT spots produced by the reaction between BF7 mAb and the antigens it recognizes within patient samples is illustrated in one embodiment of the present invention. ScanAlyze2 quantifies spots by determining the average intensity of all the pixels in the spot. Such intensity quantification provides, for each spot, a value indicating the relative expression level of the corresponding antigens in the samples. Scatter plots are generated with spot intensity values obtained in each group (cancer, benign and normal controls) using GraphPadPrism statistical package software. Then the means of the intensity of the spots in each group of interest are compared using one way ANOVA analysis of variance, with Tukey multiple comparison test. This provides all statistically significant differences in intensity means in all possible paired comparisons (p value<0.05). Area under the curve (AUC), 95% confidence intervals (CI), sensitivity and specificity of BF9 mAb are determined from receiver operating characteristic (ROC) curves. The discriminatory power of BF9 mAb in a given comparison as determined by AUC values, is not considered statistically significant when the 95% CI is large and contains 0.5 or chance line.

### Example 5: Immunodetection of BF7 Marker Polypeptides in Cancer Cell Lines

Cancer cell lines such as MDA-MB 231, and MCF7 (breast cancer); SW116, COLO 320DM, DLD-1, and WiDr (colon cancer); NCI-H157, H460 and SKLU-1 (lung cancer); PC-3, DU 145 and LNCaP (prostate cancer).

Preparation of protein extracts from cancer cell lines. Cancer cell lines (about 107) are grown in culture as recommended by ATCC provider, with 10% fetal calf serum, 100 μg/ml streptomycin and penicillin until 80% confluency, harvested, washed twice with PBS, resuspended in phosphate buffer (pH 8.0) and disrupted in the following buffer: 50 mM Tris-HCl pH 7.5, 2 mM EDTA, 100 mM NaCl, 1% NP40, and 1 mM vanadate solution containing the following protease inhibitors: PMSF, aprotinin, leupeptin at 1, 2 and 4 mM respectively. The cell lysate is centrifuged for 5 min at 14,000 rpm. Protein concentration of cancer cell extracts is determined using the BCA (bicinchoninic acid) Protein Assay Reagent Kit (Pierce, Rockford, IL) using a 1:200 dilution of extract, and a BSA standard. A microplate reader (vmax, Molecular Device) is used to read the absorbance at 570 nm. Stock solutions of protein extracts at 1 mg/ml are used.

### Example 6: Staining of Cancer Tissues by Immunohistochemistry

To demonstrate the specificity of the markers and monoclonal antibody of the present invention, and their use in histology-based diagnostic applications, tissue slides or tissue arrays displaying tissues from cancer and benign patient, and normal controls (matched, i.e. from the same patient, or unmatched) are used as follows.

5-μm formalin-fixed paraffin-embedded human tissue section slides or tissue microarrays are deparaffinized by baking slides in oven at 60° C. for 30 min followed by immersion in three xylene baths for 5 min each. Slides are rehydrated by immersion in two 100% ethanol baths for 5 min each, then in 95% ethanol, 70% ethanol baths for 3 min each, and finally soaked in water.

Endogenous peroxidase is blocked by treating slides with 3% hydrogen peroxide solution in PBS for 10 min at RT, then washing them twice in PBS for 3 min each. Antigen retrieval is obtained by heating slides in a pressure cooker at full pressure for 5 min in 10 mM Tris, 1 mM EDTA pH 9, or in Tris-sodium citrate 10 mM, 0.05% Tween 20 pH 6. Slides are then cooled to RT in the same buffer for 10-20 min, rinsed in tap water for 3 min, and finally immersed in Tris buffer.

To block endogenous biotin, which may interfere in some tissues with the detection systems, slides are incubated for 15 min at RT in a streptavidin solution in PBS (100 μg/ml), rinsed with Tris buffer, followed by incubation with biotin solution (500 μg/ml) in PBE (PBS with 1% BSA, 1 mM EDTA, 1.5 mM NaN₃ pH 7.4) for 30-60 min at RT, and washed in PBS. Non-specific binding is further blocked by treating slides for 15 min at RT in 3% horse serum diluted in PBE.

Slides are incubated with mAb BF7 (either undiluted cell culture supernatant, or appropriately diluted 1:2-1:20 in PBE buffer) for 30 min at 37° C., or 1 hr at RT or overnight at 4° C. in a humidity chamber, then rinsed 3 times for 5 min each in Tris buffer. Slides are covered with a 1:1000 dilution of biotinylated secondary antibody in PBE buffer, and incubated for 30 min at 37° C. or 1 hr at RT, then washed 3 times for 5 min each in Tris buffer. Slides are then covered with 1:1000 dilution of peroxidase-conjugated streptavidin diluted in PBE (without azide), and incubated for 30 min at 37° C. or 1 hr at RT, then washed 3 times for 5 min each in Tris buffer.

Finally, a few drops of AEC substrate solution (1 ml of 4 mg/ml AEC stock solution in DMF, plus 15 ml of 0.1 M Na acetate pH 5, and 15 μl of 30% hydrogen peroxide) are used to cover the slides. The reaction is allowed to pursue for 10-40 min, then visualized under the microscope, and stopped with tap water whenever appropriate. Slides are rinsed in water, and counterstained with a few drops of weak Mayer's hematoxylin solution for 1-2 min. Slides are then immersed in 0.1% sodium bicarbonate solution until nuclei turn blue. Slides are covered with aqueous mount media, placed in an oven at 70° C. and then let dry for 10-20 min or overnight at RT.

### Example 7: Western Blot Analysis

Total protein extracts (equivalent to 10 microgram per lane) from a given tissue or cancer cell extract are loaded, separated on polyacrylamide-SDS and transferred onto nitrocellulose membrane according to standard procedures. Different percentage of polyacrylamide may be used as known by those skilled in the art, depending on the expected molecular weight of the marker, and nitrocellulose can be replaced by PVDF, nylon membrane or other support. After transfer, the membrane is saturated for 1 hr in TNE/Tween blocking buffer (10 mM Tris-HCl pH 7.5, 2.5 mM EDTA, 50 mM NaCl, 0.1% Tween 20) containing 2.5% dried non-fat milk. The membrane is used as is or cut into strips of different size as necessary. Each membrane section or strip is first blocked with BSA or other commonly used blocking agent, then incubated for 1 hr with an antibody at appropriate dilution in the blocking buffer.

The blot is washed 5 times in the same buffer described above and incubated for 1 hr with a goat anti-mouse secondary antibody conjugated with IRDye 800CW fluorescent dye (Li-cor) according to manufacturer's instructions. Then membrane sections or strips are washed 5 times for 10 min each in TNE/Tween without milk. Antigen-antibody complexes are visualized by scanning the membrane sections or strips using the Odyssey infrared Imaging System (Li-cor) according to manufacturer's instructions. Other detection systems, known to the skilled in the art, can be used as well.

### Example 8: Detection of Secreted BF7 Markers from Cancer Cell Lines

Cancer cell lines. To test for the presence of secreted BF7 markers in cancer cell lines, cell culture supernatants were assayed with the mAb BF7.

Preparation of cell culture supernatants. Tissue culture supernatants (TCS) are centrifuged to remove cell debris and supernatants are precipitated by slow addition of 1-1.5 volumes of ice-cold acetone. Precipitation is carried out on ice or at −20° C. for 1 hr. After 15 min centrifugation at 4° C. using pre-cooled rotors, tubes are inverted to completely remove supernatants. Pellets are quickly recentrifuged to fully eliminate the last drops of supernatant. Finally pellets are allowed to dry for 5-10 min under the hood, and resuspended in 2.5 ml of Tris 50 mM pH 7. Samples are homogenized with sonicator, whenever needed, and protein concentration is measured via a BCA assay (see above). Samples are diluted to 1 mg/ml working solutions.

To prepare serum free culture medium (SFCM) to facilitate analysis of potentially secreted proteins by immunodetection analysis of cancer cell lines by MPAT, cells are grown to 70% confluency, complete medium is removed and replaced with medium without fetal calf serum and grown for 25 hr at 37° C. SFCM is then precipitated as above.

### Example 9: BF7 ELISA Assay

A typical sandwich ELISA assay using matched capture and detection mAb pair to detect BF7 in patient serum. Nunc Maxisorp immunoplate wells were coated with 100 μl capture specific mAb in PBS, and incubated overnight at 4° C. The wells were then blocked with 350 μl of blocking solution for 1 hr at 37° C. and were washed three times with PBS. Antigen source (SFCM or serum samples), calibrators and controls were added to the wells and incubated for 2 hr at RT. The wells were washed four times with PBS containing 0.05% v/v Tween 20, and 100 μl of biotinylated detecting BF7 specific mAb diluted in blocking solution was added. After incubation for 1 hr at 37° C., the wells were washed as previously described, and 100 μl of streptavidin linked horseradish peroxidase (HRP; Jackson ImmunoResearch) was added according to manufacturer instructions. Wells were then washed with PBST, incubated with 3,3′,5,5′-tetramethylbenzidine substrate solution (TMB, Sigma) for color development, and the reaction stopped with 2M HCL. The absorbance was read at 450 nm with a microplate reader (vmax, Molecular Devices).

Antigen source. A SFCM (serum free cell culture medium) of the human colon cancer cell line SW116 (ATCC CCL 233) was prepared as described (Kulasingam, 2007; Luka, 2011), and used as surrogate antigen (Ag) source. Cells were grown in SFCM to ensure that the conditioned media contained no other exogenous proteins. Multiple SFCM collections were pooled in a large batch, aliquoted in small fractions to avoid multiple freeze-thaw cycles, and stored at −80° C. A small aliquot was used to measure total protein concentration (expressed in μgEquivalent/ml) by a BCA protein assay kit (Pierce).

Antibodies. Matched capture and detection mAb pair against BF7 were protein A/G purified from mouse ascites, dialyzed against PBS, aliquoted in small volumes to minimize multiple freeze-thaw cycles and stored at −20° C. Detection mAb was also biotinylated using Sulfo-NHS-LC-biotin (Pierce) according to manufacturer's instruction. MAb concentration was determined by BCA protein assay. Biotinylated mAb was stored in small aliquots at −20° C. Studies herein were performed with the same lots of mAbs. Working concentrations of mAbs were determined by chessboard titration of both capture and detection mAb using constant concentration of antigen source. The optimal working concentration was defined as the one yielding the maximum signal to noise ratio with an acceptable non specific binding (OD<0.2) in assay diluent.

Calibrators, controls and standard curve. Zero (0) calibrator is a serum pool from normal donors (Sigma) with undetectable level of the antigen. A 10× standard calibrator stock was prepared by mixing a known quantity of BF7 antigen source with the zero calibrator. In addition, two positive controls were prepared by pooling serum samples from breast cancer patients, previously identified as having high or low amount of BF7 marker levels. Large volumes of 10× standard calibrator stock, zero calibrator and positive controls were aliquoted and stored at −80° C. On the day of the assay, unknown serum samples, positive controls, zero and standard calibrators were diluted 1:10 in assay diluent (to 10% final serum concentration). A standard curve is constructed by measuring the absorbance of a serially diluted 1× standard calibrator using the 10% zero calibrator as diluent. Each assay plate contains standard curve calibrators, positive controls and unknown samples. Measurements of calibrators, controls and unknowns were done in duplicate, and the average was taken as final reading. Unknown samples producing ODs outside the range of the standard curve were re-assayed at an appropriate dilution until they fit the range of the standard curve. BF7 level (μgE/ml) in patient serum was interpolated from the standard curve generated by 4 parameter logistic model (R²=0.992).

Assay quality control. To ensure reproducibility between different assay runs, standard curves were plotted as ODs normalized between maximal and minimal values obtained on the same microplate as a function of Log [Ag concentration]. Statistical comparison of multiple standard curves was done using nonlinear regression curve fit (GraphPad Prism7.0). Midpoint antigen concentration (C50) values were calculated from the sigmoid curves and did not show statistically significant difference (p>0.05; F-test). If the results of the assay did not fit the established acceptable range of controls values, they were considered invalid.

Analytical validation. To examine the analytical dilution recovery, a known amount of BF9 surrogate antigen is spiked into assay diluent supplemented with 10% of a normal human serum pool containing undetectable levels of said antigen (zero calibrator). The sample is then serially diluted in assay diluent, in quadruplicate samples, as indicated. In each of the 5 dilutions tested, the measured antigen concentration is derived from the standard calibration curve, and the average measured concentration of quadruplicate values (SD) is plotted against the expected concentration. Then the ratio between the expected antigen concentration and the average measured concentration is calculated thus yielding a % recovery. Each % recovery result is within the acceptable range of 80-120%. The percent coefficient variation (% CV) is defined as the ratio of the standard deviation (SD) over the mean×100 (SD/mean*100). % CV of quadruplicate values are within acceptable limits (<15%). Dilution linearity is defined by a regression coefficient R² value of ˜1. Regression analysis yields a slope of 0.971 and R²=0.999, indicating linear dilution in the range of concentrations tested. To measure the analytical spiking recovery, a normal human serum pool diluted 1:10 in assay diluent containing undetectable levels of antigen, is spiked, in quadruplicate, with 5 amounts of BF9 surrogate antigen, ranging from 1.07 to 17.1 μgE/ml. Recovered antigen concentration is measured and interpolated using the BF9 calibration curve. Quadruplicate values of each measured concentration are averaged. % recovery and % CV are calculated. To determine the inter-assay reproducibility, the spiking recovery experiment using 5 different concentrations of antigen was repeated in triplicate and in 3 independent days to measure inter-assay variance. % recovery and % CV of 9 measurements are within acceptable limits.

### Example 10: Statistical Analysis of Data

Statistical analysis, ROC curves and scatter plots were performed with GraphPad Prism7.0 (GraphPad Software, Inc., La Jolla, CA). The median biomarker concentration values in each group were compared to determine whether differences were statistically significant (p<0.05). The relationship between clinicopathological parameters and serum biomarker levels was examined as well (Table 1). Statistical analysis of two-group and multi-group comparisons was carried out using the non-parametric Mann-Whitney U test and the Kruskal-Wallis test, respectively. Receiver operating characteristic (ROC) curves were calculated to evaluate the diagnostic performance of the BF7 assay in various comparisons, by determining sensitivity (SE), specificity (SP), area and curve (AUC), and confidence interval (CI). For statistical analysis of age in different groups (when distribution passed D'Agostino & Pearson normality test) we compared the age means±SD using one-way ANOVA followed by Dunn's multiple comparison test.

### Example 11: Xenograft Models

To demonstrate the proliferative effect of BF7 marker polypeptides, a suspension of 5×106 SW1116 colorectal cancer cells in PBS was injected s.c. in each of 4 male athymic NCR nu/nu mice 7 to 8 weeks of age. Standard protocols were used (Morton, 2007). Tail vein blood (200 μl) was drawn from each mouse prior to injection, and every week thereafter, and corresponding serum was assayed in duplicate to measure BF7 levels. Tumor growth was visually inspected. Animals were kept in a pathogen-free animal facility and maintained according to PHS guidelines on Animal Welfare Assurance.

### Example 12: Clinical Samples (Serum)

Clinical samples were carefully selected to address two major clinical applications: colorectal cancer (CRC) early detection and monitoring. We used three independent serum sample sets described below. For measurement of BF7 diagnostic performance, particularly in early stage (Clinical Sample Set 1), serum samples were retrospectively obtained from the Cooperative Human Tissue Network (CHTN; Jewell, 2001) from patients with CRC and advanced adenoma. Procurement from the CHTN excluded samples with bacterial or viral infection, prior history of cancer, prior chemo or radiation treatment, recurrent cancer. All races and gender were included and represented according to the patient population within the CHTN. All samples were collected after CRC or adenoma diagnoses were made, based on colonoscopy and histopathological evaluation, and prior to surgery. All samples had well-annotated pathology report. Clinical Sample Set 1 comprised 275 pre-operative primary CRC (all four TNM stages; 137 stage I/II, 130 late stage, 8 unknown; with 50% stage 0-II), 25 polyps of which 21 advanced adenoma>1 cm based on histopathological report (Bond, 2000; Fleming, 2012) and 19 polyps with unknown size or histopathological features), and 69 healthy controls. Healthy controls, defined as individuals without any past or present history of colorectal lesions or diseases (e.g. small adenomas, hyperplastic polyps, cancer), were free of bowel disease while some presented with chronic conditions of their age group (such as hyperlipidemia) and were obtained in part form the CHTN. Clinicopathological features of patient samples from cases and controls within Clinical Sample Set 1 are summarized in Table 1. A breakdown of age and gender is provided, whenever available.

The CHTN (Jewell, 2002; https://www.chtn.org) is a network of institutions and clinical centers throughout the US. All CHTN samples are collected under harmonized SOPs, and encompass a broad patient population, thus minimizing bias due to single sample source. CHTN samples were provided with highly annotated path report, including disease condition, cancer stage and histological type, age, gender, race, prior personal and family cancer clinical history, information regarding diseases other than cancers and possible medications. Samples were provided with no private identifiers.

The second set of serum samples (referred to as Clinical Sample Set 2) addressing the detection of CRC relapse in a cross-sectional study was obtained from a third party clinical laboratory hospital specialized in CRC, and comprises 397 CRC patients who suffered a recurrence after primary cancer and with known CEA levels measured at hospital visit (AdviaCentaur assay, Siemens).

The third set of clinical samples (referred to as Clinical Sample Set 3) used for the longitudinal study comprised 347 serum samples that had been serially collected from 95 patients and obtained retrospectively from the U-CAN biobank at the University of Uppsala, Sweden (Glimelius, 2017). 4 patients were found not to have had samples taken either before or after recurrence, which made them unsuited for this study and were thus excluded. That left 339 samples from 91 patients with confirmed cases of local and distant recurrence (2 to 8 time points per patient; ranging from 54 months before and 29 months after diagnosis of recurrence). The cohort is comprised of 49 male and 42 female with a mean age of 67 years old (range 31-88). Samples have extensive well-annotated clinical information. Clinicopathological features of follow-up samples are provided in Table 3 together with breakdown by CRC stage, patient number, recurrence type (loco-regional or distant), distant recurrence site (lung, liver, or other), and time to recurrence. All patients were diagnosed with adenocarcinoma.

All samples used in this study were received on dry ice and immediately stored at −80° C. until their use, then prior to assay, samples are aliquoted on ice to avoid multiple freeze-thaw cycles. Sample quality control involved verification of clinical information and path report, and removal of samples with evidence of excess lipids, or hemolysis. All patient samples and clinical information were collected under strict human subject guidelines, with IRB-approved protocol and patient informed consent in place at the original institutions. Samples were handled, and packaged for transport by providers according to US and EU regulations, and provided to Applicant in a de-identified manner.

### Example 13: Immunodetection of Secreted BF7 Markers in Urine

Clinical samples. The clinical sample set for the urine experiment comprised 47 samples as follows: 10 colon cancer patients (6 stage I, 4 stage II), 5 with inflammatory conditions of the colon, 4 with benign conditions of the colon, 13 prostate cancer patients (11 stage II, and 2 stage III), 5 pancreatic cancer patients (4 stage II and 1 unknown), and 10 normal controls. Note that cancer stages I and II are defined as “early”, while stages III and IV are defined as “late”.

Preparation of urine samples. Unprecipitated urine samples are first centrifuged to remove debris whenever turbid, then 250 nanoliters of each urine sample are printed on the MPAT either “as is” or upon dilution 1:2 or 1:10 in Tris-Triton buffer.

MPAT: After spotting on the MPAT membrane, samples are probed with the BF7 monoclonal antibody by incubation for 30 min, followed by six washes, processed and visualized as described in Example 3.

A BF7 test assessment in an individual can be translated to an assessment of cancer for the individual, including a score or other identifier that indicates whether an individual has cancer or that indicates a certain likelihood that the individual has cancer or that identifies additional known markers for disease initiation, progression, metastasis or any other characteristic of neoplastic disorders. Similarly, the score or other identifier may indicate a specific type of cancer assessment, such as the assessments of various cancer characteristics described herein, including (but not limited to), determination of whether an individual's cancer is metastasized, determination of the stage of an individual's cancer (such as distinguishing between stage I and stage III cancer), determination of whether an individual's cancer is a malignant tumor or a benign lesion, and determining tumor regression and/or recurrence.

As noted above, the invention includes methods for diagnosing diseases having differential expression of BF7 marker polypeptides. For example, normal, control, or standard values (e.g., that represent typical expression levels of a protein in healthy individuals) for BF7 biomarkers can be established in various assay formats, such as by combining body fluids, tissues, or cell extracts taken from a patient with specific antibodies to a protein under conditions for complex formation. Standard values for complex formation in normal and disease tissues can be established by various methods, such as photometric means. Complex formation, as it is expressed in a test sample, can be compared with the standard values for correlation to disease. Deviation from a normal standard and toward a disease standard can provide parameters for disease diagnosis or prognosis while deviation away from a disease standard and toward a normal standard can be used to evaluate treatment efficacy. Alternately, threshold levels of disease or normal are established.

Platform immunological methods for detecting and measuring complex formation as a measure of the expression of BF7 biomarkers using either specific polyclonal or monoclonal antibodies are known in the art. Examples of such techniques include ELISAs, radio-immunoassays (Ms), flow cytometry (also referred to as fluorescence-activated cell sorting, or FACS), and antibody arrays.

For example, ELISA can be used to detect or quantify BF7 markers. In certain exemplary ELISA methods, an antibody that specifically binds to one such marker may be coated to the well of a suitable container (e.g., a 96 well microliter plate), a patient sample (e.g., a serum sample) can be added to the well and incubated for a period of time, and the presence of said marker in the patient sample can be detected upon binding of an epitope on a BF7 polypeptide in the patient sample to the antibody that is coated to the well. In this instance, a second antibody conjugated to a detectable moiety may optionally be added following the addition of the patient sample to the coated well. ELISA methods such as these may be modified or optimized as desired.

Further, instead of coating the well with the BF7 mAb, BF7 markers may be coated to the well. Thus, in certain ELISA methods, a BF7 polypeptide is coated to the well of a suitable container (e.g., a 96 well microliter plate), a BF7 mAb (which may optionally be conjugated to a detectable moiety such as an enzymatic substrate (horseradish peroxidase or alkaline phosphatase)) is added to the well and incubated for a period of time, and the presence of BF7 marker is detected. An antibody to BF7, whether the BF7 mAb or another, does not have to be conjugated to a detectable moiety; for example, a second antibody (which recognizes the antibody to BF7 or the BF7 mAb disclosed herein) is conjugated to a detectable moeity added to the well.

These assays and their quantitation against purified, labeled standards are well known in the art (Ausubel, supra, unit 10:1-10.6). For example, a two-site, monoclonal-based immunoassay utilizing antibodies reactive to two non-interfering epitopes can be utilized, and competitive binding assay can also be utilized (Pound (1998) Immunochemical Protocols, Humana Press, Totowa N.J.).

For diagnostic applications, an antibody can be labeled with a detectable moiety (interchangeably referred to as a “label” or “detectable substance”), such as to facilitate detection by various imaging methods. Methods for detection of labels include, but are not limited to, fluorescence, light, confocal, and electron microscopy; magnetic resonance imaging and spectroscopy; fluoroscopy, computed tomography and positron emission tomography. Numerous detectable moieties are available for labeling antibodies, including, but not limited to: 1) radioisotopes, such as 36S, 14° C., 1251, 3H, and 1311, 2) fluorescent labels such as rare earth chelates (europium chelates) or fluorescein and its derivatives, rhodamine and its derivatives, dansyl, Lissamine, phycoerythrin and Texas Red, 3) enzyme-substrate labels (e.g., U.S. Pat. Nos. 4,275,149 and 4,318,980).

BF7 can be detected in vivo in an individual patient by introducing into the patient a labeled antibody (or other type of detection agent) specific for the protein marker. For example, an antibody can be labeled with a radioactive marker as described above whose presence and location in an individual can be detected by standard imaging techniques.

The present invention includes the detection of any BF7 marker in the form of polypeptide or polynucleotide, or any combination of 2, 3, 4, or more from the group Group 1 consisting of Sterile alpha motif domain-containing protein 14 (SAMD14, SEQ ID NO: 1); Nuclear pore complex protein Nup107 (NUP107, SEQ ID NO: 2); Putative IQ motif and ankyrin repeat domain-containing protein LOC642574 (LOC642574, SEQ ID NO: 6); Transcription initiation factor TFIID subunit 4 (TAF4, SEQ ID NO: 9); Protein Bassoon (BSN, SEQ ID NO: 20);

The present invention includes the detection of any BF7 marker in the form of polypeptide or polynucleotide, or any combination of 2, 3, 4, or more from the Group 2 consisting of Probable rRNA-processing protein EBP2 (EBNA1BP2, SEQ ID NO: 3); FH1/FH2 domain-containing protein 1 (FHOD1, SEQ ID NO: 5); UPF0488 protein C8orf33 (C8orf33, SEQ ID NO: 7); 28S ribosomal protein S11, mitochondrial (MRPS11, SEQ ID NO: 8); Serrate RNA effector molecule homolog (SRRT, SEQ ID NO: 10); 40S ribosomal protein S13 (RPS13, SEQ ID NO: 11); 60S ribosomal protein L4 (RPL4, SEQ ID NO: 12); Voltage-gated potassium channel subunit beta-3 (KCNAB3, SEQ ID NO: 13); Kelch-like protein 14 (KLHL14, SEQ ID NO: 15);

The present invention includes the detection of any BF7 marker in the form of polypeptide or polynucleotide, or any combination of 2, 3, 4, or more from the Group 3 consisting of Transcriptional activator MN1 (MN1, SEQ ID NO: 4); Plakophilin-3 (PKP3, SEQ ID NO: 14); Ras-related protein Rab-27B (RAB27B, SEQ ID NO: 16); 6-phosphogluconolactonase (PGLS, SEQ ID NO: 17); Cyclin L2 (CCNL2, SEQ ID NO: 18); Iron-sulfur protein NUBPL (NUBPL, SEQ ID NO: 19); Histone H1.2 (HIST1H1C, SEQ ID NO: 21); Cytosolic Fe—S cluster assembly factor (NUBP2, SEQ ID NO: 23); Rho-related GTP-binding protein RhoU (RHOU, SEQ ID NO: 24); Collagen alpha-1 (I) chain (COL1A1, SEQ ID NO: 25); Tetraspanin (TSPAN7, SEQ ID NO: 27); Ras-related protein RAB-11B (RAB11B, SEQ ID NO: 28);

The present invention includes the detection of any BF7 marker in the form of polypeptide or polynucleotide, or any combination of 2, 3, 4, or more from the Group 4 consisting of Ubiquilin 4 (UBQLN4, SEQ ID NO: 22); Galectin-3 binding protein (LGALS3BP, SEQ ID NO: 26).

The present invention further includes any BF7 marker in the form of polypeptide or polynucleotide, or any combination, of 2, 3, 4, or more from the group consisting of Group 1 and/or Group 2 with any BF7 marker in the form of polypeptide or polynucleotide, or any combination, of 2, 3, 4, or more from the group consisting of Group 3 and/or Group 4 and/or Group 5.

Group 5 comprises the existing markers including but not limited to Ki-67 (Ki-67), P16INK4a (p16), Estrogen receptor (ER-alfa), Progesterone receptor (PR), c-erbB-2 (HER-2), soluble HER2, Cathepsin D, CA15-3 (CA15-3), CA27.29 (CA27.29), Carcinoma embryonic antigen (CEA), Vimentin (Vimentin), Prostate specific antigen (PSA), Prostatic acid phosphatase (PAP), Kallikrein-2 (KLK-2), p504S (p504S), Tumor Protein p63 (p63), Chromogranin A (CgA), Progastrin releasing peptide type 3 (ProGRP), Neuron specific enolase (NSE), Melanocyte lineage-specific antigen (Gp100), MART-1 (MART-1), MAGE-1 (MAGE-1), Calcium binding protein A4/Metastasin 100 (S100A4), Alfa-fetoprotein (AFP), Macrophage inhibitory cytokine (MIC-1), Osteopontin (OSPN), CA19-9 (CA19-9), Mucin-16/ovarian carcinoma antigen CA-125 (CA-125), Leukocyte common antigen (CD45 LCA), CD68 (CD68), Cytokeratins 5, 6 (CK5/6), Cytokeratin 16, 17 and 18 (CK16/17/18), Cytokeratin 17 (CK17), Cytokeratin 19 fragment/CYFRA 21.1, B-cell lymphoma-2 (BCL-2), B-Lymphocyte antigen (CD20), Hematopoietic progenitor CD34 (CD34), Proto-oncogene P53 (p53), Mucin-2 (MUC-2), Mucin-3A (MUC-3), Mucin-4 (MUC-4), Mucin 5AC (MUC-5AC), Mucin-6 (MUC-6), Proliferating cell nuclear antigen (PCNA), Tyrosinase (Tyr), Prostate specific membrane antigen (PSMA-1), Calcium binding protein (S1002), Tissue inhibitor of metalloproteinase (TIMP-1), Squamous cell carcinoma antigen (SCC), Androgen Receptor (ARC), Urokinase plasminogen activator (UPA), Plasminogen activator inhibitor (PAI), Protein uncharacterized ENSP0381381, CA-242, CYFRA21-1.

A description of the use and potential applicability of the markers to the present invention is provided at the following which are incorporated by reference. Duffy MJ, Esteva FJ, Harbeck N, Hayes DF, Molina R. Tumor markers in breast cancer, In: National Academy of Clinical Biochemistry (NACB) Laboratory Medicine Practice Guidelines “Use of Tumor Markers in testicular, prostate, colorectal, breast and ovarian cancer”, Sturgeon CM, Diamandis EP, Ed., Chapter 5, pp 37-49, 2009; Harris L et al., American Society of Clinical Oncology, Update of Recommendations for the Use of Tumor Markers in Breast Cancer, J Clin Oncol 25 (33): 5287-5312, 2007; see also for Goggins M, Koopmann J, Yang D, Canto MI, Hruban RH. National Academy of Clinical Biochemistry (NACB) Laboratory Medicine Practice Guidelines for the Use of Tumor Markers in Pancreatic Ductal Adenocarcinoma. www.nacb.org/tumors, 2005; see also for Brunner N, Duffy MJ, Haglund C, Holten-Andersen M, Nielsen HJ. Tumor markers in colorectal malignancy, In: National Academy of Clinical Biochemistry (NACB) Laboratory Medicine Practice Guidelines “Use of Tumor Markers in testicular, prostate, colorectal, breast and ovarian cancer”, Sturgeon CM, Diamandis EP, Ed., Chapter 4, pp 27-35, 2009; Locker GY, Hamilton S, Harris J, Jessup JM, Kemeny N, Macdonald JS, Somerfield MR, Hayes DF, Bast RC Jr., ASCO Tumor Panel Expert Panel. ASCO 2006 update of recommendations for the use of tumor markers in gastrointestinal cancer. J Oncol, November 20; 24 (33): 5313-27, 2006; see also for Stieber P, Hatz R, Molina R, von Pawel J, Schalhorn A, Schneider J, Yamaguchi K. Tumor markers in lung cancer, In: National Academy of Clinical Biochemistry (NACB) Laboratory Medicine Practice Guidelines “Use of Tumor Markers in testicular, prostate, colorectal, breast and ovarian cancer”, Sturgeon CM, Diamandis EP, Ed., 2006; see also for Chan DW, Bast RC Jr, Shih I-M. Sokoll L, Soletormos G. Tumor markers in ovarian cancer, In: National Academy of Clinical Biochemistry (NACB) Laboratory Medicine Practice Guidelines “Use of Tumor Markers in testicular, prostate, colorectal, breast and ovarian cancer”, Sturgeon CM, Diamandis EP, Ed., Chapter 6, pp 51-59, 2009; see also for Lilja H, Semjonow A, Sibley P, Babaian R, Dowell B, Rittenhouse H, Sokoll L. R. Tumor markers in prostate cancer, In: National Academy of Clinical Biochemistry (NACB) Laboratory Medicine Practice Guidelines “Use of Tumor Markers in testicular, prostate, colorectal, breast and ovarian cancer”, Sturgeon CM, Diamandis EP, Ed., Chapter 3, pp 15-25, 2009.

BF7 assays are provided that have at least 70% sensitivity at 95% specificity, or at least 70% specificity at 95% sensitivity. In certain embodiments, BF7 assays are provided that have at least 85% sensitivity at 95% specificity, or at least 85% specificity at 95% sensitivity. In further embodiments, BF7 assays are provided that have at least 90% sensitivity or at least 90% specificity or that have at least 95% sensitivity or at least 95% specificity. In yet further embodiments, assays are provided that have at least 70, 75, 80, 85, 90, 91, 92, 93, 94, 95, 96, 97, 98, or 99% (or any other percentage in-between) sensitivity and 70, 75, 80, 85, 90, 91, 92, 93, 94, 95, 96, 97, 98, or 99% (or any other percentage in-between) specificity. In yet further embodiments, BF7 assays are provided that have at least 0.7, 0.75, 0.8, 0.85, 0.9, 0.91, 0.92, 0.93, 0.94, 0.95, 0.96, 0.97, 0.98, or 0.99 (or any other value in-between).

BF7 assay devices for detection of BF7 biomarkers can be provided in the form of kits, such as for use in performing the methods disclosed herein. Furthermore, any kit can contain one or more detectable labels (e.g., detactably labeled reagents such as antibodies), such as a fluorescent moiety, etc. A kit can comprise (a) reagents comprising at least one antibody for detecting BF7 markers, and optionally (b) known markers specific for cancer or a type of cancer of interest.

For immunohistochemistry, a disease tissue sample may be, for example, fresh or frozen or may be embedded in paraffin and fixed with a preservative such as formalin. A fixed or embedded section can be contacted with a labeled primary BF7 antibody and secondary antibody, wherein the antibody is used to detect the expression of BF7 proteins in situ.

Antibodies can be used to detect BF7 polypeptide markers in situ, in vitro, or in a cell lysate or supernatant in order to evaluate the abundance and pattern of expression. Also, antibodies can be used to assess abnormal tissue distribution or abnormal expression during development or progression of a biological condition. Antibodies against BF7 markers are useful for detecting the presence of the proteins in cells or tissues to determine the pattern of expression of the proteins among various tissues in an organism and over the course of the organism's development.

Further, mAb BF7 is used to assess expression in disease states such as in active stages of a disease or in an individual with a predisposition toward disease related to the proteins' function. When a disorder is caused by inappropriate tissue distribution, developmental expression, or level of expression of BF7 markers, an antibody can be prepared against the normal protein. If a disorder is characterized by a specific mutation in a BF7 protein, antibodies specific for the mutant protein can be used to assay for the presence of the specific mutant.

In certain embodiments, the invention provides detection or diagnostic methods of BF7 markers using LC/MS. The differential expression of marker polypeptides detected by mAb BF7 in disease and healthy (or drug-resistant and drug-sensitive, for example) samples can be quantitated using mass spectrometry and ICAT (Isotope Coded Affinity Tag) labeling, which is known in the art. ICAT is an isotope label technique that allows for discrimination between two populations of proteins, such as a healthy and a disease sample. BF7 over-expression or under-expression, as measured by ICAT, can indicate, for example, the likelihood of having or developing a disease or an associated pathology.

LC/MS spectra can be correlated to disease and normal samples and processed as follows. The raw scans from the LC/MS instrument can be individualized for peak detection and to isolate sequence information using signal/noise reduction software. Filtered peak lists can then be used to detect ‘features’ corresponding to specific BF7 polypeptides from the original sample(s). Features are characterized by their mass/charge ratio, charge, intensity, retention time, isotope pattern, sequence, for example through labeled residue sequencing to determine examples of the BF7 polypeptide sequences herein (SEQ ID NO: 1 to SEQ ID NO: 31) to separate disease from normal.

The BF7 related signal intensity present in both healthy and disease samples can be used to calculate the differential expression, or relative abundance, of the polypeptide. The intensity of a peptide found exclusively in one sample can be used to calculate a theoretical expression ratio for that peptide. Expression ratios can be calculated for each peptide in an assay or experiment.

Natural or synthetic polynucleotides are useful as hybridization probes for determining the presence, level, form, and/or distribution of BF7 nucleic acid expression. Exemplary probes can be used to detect the presence of, or to determine levels of, a specific nucleic acid molecule in cells, tissues, and in organisms. Accordingly, probes corresponding to BF7 related sequences as described herein can be used to assess expression and/or gene copy number in a given patient sample, cell, tissue, or organism, which can be applied to, for example, diagnosis of disorders involving an increase or decrease in corresponding BF7 protein expression relative to normal BF7 protein expression levels.

Nucleic acid test kits for detecting the presence of natural BF7 polynucleotides (e.g., mRNA or genomic DNA) in a biological sample comprise reagents such as a labeled or labelable nucleic acid or agent capable of detecting BF7 polynucleotides in a biological sample and means for comparing the amount of BF7 polynucleotide in the sample with a standard.

Detection of mutations such as deletions, additions, or substitutions of one or more nucleotides in a gene, chromosomal rearrangements (such as inversions or transpositions), and modification of genomic DNA such as aberrant methylation patterns or changes in gene copy number or amplification can be detected at the nucleic acid level by a variety of techniques. For example, genomic DNA or RNA from a patient or group of patients can be analyzed directly or can be amplified (e.g., using PCR) prior to analysis. In certain exemplary embodiments, detection of a mutation involves the use of a probe/primer in a PCR reaction (see, e.g. U.S. Pat. Nos. 4,683,195 and 4,683,202), such as anchor PCR or RACE PCR, or, alternatively, in a ligation chain reaction (LCR) (see, e.g., Landegran et al., Science 241:1077-1080 (1988) and Nakazawa et al., PNAS 91:360-364 (1994)), the latter of which can be particularly useful for detecting point mutations in a gene (see Abravaya et al., Nucleic Acids Res. 23:675-682 (1995)). Exemplary methods such as these can include the steps of collecting a biological sample from a patient, isolating nucleic acid (e.g., genomic, mRNA, or both) from the cells of the sample, contacting the nucleic acid with one or more primers which specifically hybridize to a marker nucleic acid under conditions such that hybridization and amplification of the marker nucleic acid (if present) occurs, and detecting the presence or absence of an amplification product, or defecting the size of the amplification product and comparing the length to a control sample. Deletions and insertions can be detected by a change in size of the amplified product compared to a normal genotype. Point mutations can be identified by hybridizing amplified DNA to normal RNA or antisense DNA sequences, for example.

Alternatively, mutations in BF7 polynucleotides can be identified, for example, by alterations in restriction enzyme digestion patterns as determined by gel electrophoresis. Further, sequence-specific ribozymes (U.S. Pat. No. 5,498,531) can be used to identify the presence of specific mutations by development or loss of a ribozyme cleavage site. Perfectly matched sequences can be distinguished from mismatched sequences by nuclease cleavage digestion assays or by differences in melting temperature.

Sequence changes at specific locations can be assessed by nuclease protection assays such as RNase or chemical cleavage methods. Furthermore, sequence differences between a mutant BF7 gene and a corresponding wild-type gene can be determined by direct DNA sequencing. A variety of automated sequencing procedures can be utilized when performing diagnostic assays (Naeve, C. W., (1995) Biotechniques 19:448), including sequencing by mass spectrometry (e.g., PCT International Publication No. WO 94/16101; Cohen et al., Adv. Chromatogr. 36:127-162 (1996); and Griffin et al., Appl. Biochem. Biotechnol. 38:147-159 (1993)).

Methods for detecting mutations in a BF7 polynucleotide also include methods in which protection from cleavage agents is used to detect mismatched bases in RNA/RNA or RNA/DNA duplexes (Myers et al., Science 230:1242 (1985)); Cotton et al., PNAS 85:4397 (1988); Saleeba et al., Meth. Enzvmol. 217:286-295 (1992)), electrophoretic mobility of mutant and wild type nucleic acid is compared (Orita et al., PNAS 86:2766 (1989); Cotton et al., Maw. Res. 285:125-144 (1993); and Hayashi et al., Genet. Anal. Tech. Appl. 9:73-79 (1992)), and movement of mutant or wild-type fragments in polyacrylamide gels containing a gradient of denaturant is assayed using denaturing gradient gel electrophoresis (DGGE) (Myers et al., Nature 313:495 (1985)). Examples of other techniques for detecting point mutations include selective oligonucleotide hybridization, selective amplification, and selective primer extension.

Natural and synthetic molecules of the invention are also useful for monitoring the effectiveness of modulating agents on the expression or activity of BF7 marker polypeptides, such as in clinical trials or in a treatment regimen. For example, the gene expression pattern of a BF7 natural polynucleotide expression or the presence or amounts of a BF7 marker can serve as a barometer for the continuing effectiveness of treatment. The gene expression pattern can also serve as a marker indicative of a physiological response such as resistance or sensitivity of the cancer cells to the compound. For example, based on monitoring nucleic acid expression, the administration of a compound can be increased or alternative compounds to which the patient has not become resistant can be administered.

In one embodiment, the level of BF7 mRNA is determined either by in situ and by in vitro formats in a biological sample using methods known in the art. Many expression detection methods use isolated RNA. For in vitro methods, any RNA isolation technique that does not select against the isolation of mRNA can be utilized for the purification of RNA from tumor, tissue samples, or tissue cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The mRNA is used in hybridization or amplification assays that include, but are not limited to, Southern or Northern analyses, polymerase chain reaction analyses and probe arrays. One preferred diagnostic method for the detection of mRNA levels involves contacting the mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a marker of the present invention. Other suitable probes for use in the diagnostic assays of the invention are described herein. Hybridization of a BF7 mRNA with the probe indicates that a BF7 marker is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods of several formats having probes linked to a variety of detection systems (such as radioactive or fluorescent probes) for use in detecting the level of BF7 species.

For in situ methods, BF7 mRNA need not be isolated from the tissue or tumor cells prior to detection. In such methods, a cell or tissue sample is prepared/processed using known histological methods. The sample is then immobilized on a support, typically a glass slide, and then contacted with a probe that can hybridize to a given BF7 mRNA.

The invention also includes vectors and host cells containing natural and synthetic BF7 nucleic acid molecules. The term “vector” refers to a vehicle, such as a nucleic acid molecule, which can transport BF7 polynucleotides. When the vector is a nucleic acid molecule, the BF7 polynucleotides are covalently linked to the vector nucleic acid to yield a synthetic polynucleotide. A vector can be, for example, a plasmid, single or double stranded phage, a single or double stranded RNA or DNA viral vector, a mini-locus or artificial chromosome, such as a BAC, PAC, YAC, or MAC. A vector can be maintained in a host cell as an extrachromosomal element such as a plasmid where it replicates and produces additional copies of BF7 polynucleotides or the vector can integrate into the host cell genome and produce additional copies of BF7 polynucleotides when the host cell replicates.

Vectors of the invention include maintenance (cloning vectors) and vectors for expression (expression vectors) of the nucleic acid molecules, for example. Expression vectors can express a portion of, or all of, a protein sequence. Vectors can function in prokaryotic or eukaryotic cells or in both (shuttle vectors). Vectors also include insertion vectors, which integrate a nucleic acid molecule into another nucleic acid molecule, such as into the cellular genome (such as to alter in situ expression of a gene and/or gene product). For example, an endogenous protein-coding sequence can be entirely or partially replaced via homologous recombination with a variant of the protein-coding sequence containing one or more specifically introduced mutations.

Expression vectors can contain cis-acting regulatory regions that are operably linked in the vector to a BF7 polynucleotide such that transcription of the polynucleotide is allowed in a host cell. BF7 polynucleotides can be introduced into the host cell with a separate nucleic acid molecule capable of affecting transcription. The separate nucleic acid molecule may provide, for example, a trans-acting factor interacting with the cis-regulatory control region to allow transcription of the nucleic acid molecules from the vector. Alternatively, a trans-acting factor may be supplied by a host cell. Additionally, a trans-acting factor can be produced from a vector itself.

Regulatory sequences to which BF7 nucleic acid molecules can be operably linked include, for example, promoters for directing mRNA transcription. These include, but are not limited to, the left promoter from T7 bacteriophage promoter, the lac, TRP, and TAC promoters from E. coli, the early and late promoters from SV40, the CMV immediate early promoter, the adenovirus early and late promoters, and retrovirus long-terminal repeats.

In addition to control regions that promote transcription, expression vectors can also include regions that modulate transcription, such as repressor binding sites and enhancers. Examples include the SV40 enhancer, the cytomegalovirus immediate early enhancer, polyoma enhancer, adenovirus enhancers, and retrovirus enhancers.

In addition to containing sites for transcription initiation and control, expression vectors can also contain sequences necessary for transcription termination and, in the transcribed region, a ribosome binding site for translation. Other regulatory control elements for expression include translation, initiation, and termination codons as well as polyadenylation signals. Numerous regulatory sequences useful in expression vectors are well known in the art (e.g., Sambrook et al., Molecular Cloning: A Laboratory Manual. 3rd. ed., Cold Spring Harbor Laboratory Press, Cold Spring Harbor, N.Y. (2001)).

A variety of expression vectors can be used to express a BF7 polynucleotide. Such vectors include chromosomal, episomal, and virus-derived vectors, for example vectors derived from bacterial plasmids, from bacteriophage, from yeast episomes, from yeast chromosomal elements, including yeast artificial chromosomes, from viruses such as baculoviruses, papovaviruses such as SV40, Vaccinia viruses, adenoviruses, poxviruses, pseudorabies viruses, and retroviruses. Vectors may also be derived from combinations of these sources such as those derived from plasmid and bacteriophage genetic elements, e.g. cosmids and phagemids. Appropriate cloning and expression vectors for prokaryotic and eukaryotic hosts are described in Sambrook et al., Molecular Cloning: A Laboratory Manual. 3rd. ed., Cold Spring Harbor Laboratory Press, Cold Spring Harbor, N.Y. (2001). Bacterial cells include, but are not limited to, E. coli, Streptomyces, and Salmonella typhimurium. Eukaryotic cells include, but are not limited to, yeast, insect cells such as Drosophila, animal cells such as COS and CHO cells (e.g., DG44 or CHO-s), and plant cells.

A regulatory sequence can provide constitutive expression in one or more host cells (e.g., tissue specific) or can provide for inducible expression in one or more cell types such as by temperature, nutrient additive, or exogenous factors such as a hormone or other ligand. A variety of vectors providing for constitutive and inducible expression in prokaryotic and eukaryotic hosts are well known in the art.

Recombinant host cells can be prepared by introducing vector constructs, such as described herein, into cells by techniques readily available to a person of ordinary skill in the art. These techniques include, but are not limited to, calcium phosphate transfection, DEAL-dextran-mediated transfection, cationic lipid-mediated transfection, electroporation, transduction, infection, lipofection, microinjection, and other techniques such as those found in Sambrook, et al. (Molecular Cloning: A Laboratory Manual. 3rd. ed., Cold Spring Harbor laboratory Press, Cold Spring Harbor, N.Y. (2001)).

For example, using techniques such as these, a retroviral or other viral vector can be introduced into mammalian cells. Examples of mammalian cells into which a retroviral vector can be introduced include, but are not limited to, primary mammalian cultures or continuous mammalian cultures, COS and CHO cells, NIH3T3, 293 cells (ATCC #CRL 1573), and dendritic cells.

Host cells can contain more than one vector. Thus, different polynucleotide sequences can be introduced on different vectors of the same cell. Similarly, BF7 polynucleotides can be introduced either alone or with other unrelated nucleic acid molecules such as those providing trans-acting factors for expression vectors. When more than one vector is introduced into a cell, the vectors can be introduced independently, co-introduced, or joined to the nucleic acid molecule vector.

Bacteriophage and viral vectors can be introduced into cells as packaged or encapsulated virus by standard procedures for infection and transduction. Viral vectors can be replication-competent or replication-defective. If viral replication is defective, replication can occur in host cells that provide functions that complement the defects.

If secretion of BF7 markers from a host cell is desired, appropriate secretion signals can be incorporated into the vector harboring the expression sequence for that marker. The signal sequence can be endogenous or heterologous to the protein.

Recombinant host cells that express BF7 polypeptides or any variant thereof have a variety of uses. For example, such host cells are useful for producing BF7 variants, which can be further purified to produce desired amounts of the protein or fragments thereof. Thus, host cells containing expression vectors are useful for protein production or for conducting cell-based assays for BF7 polypeptide expression.

### Predictive Medicine

The present invention pertains to the field of predictive medicine in which diagnostic assays, prognostic assays, pharmacokinetics, and pharmacogenomics are used for prognostic (predictive) purposes to identify an asymptomatic patient or patient population or to propose course of for treatment in monitoring a cancer patient undergoing testing or treatment. Accordingly, the present invention includes the process of implementing a protocol for future use of markers for monitoring the progress of a patient or group of patients following a first screening, a first diagnosis, a plurality of additional, subsequent screenings or diagnose or treatment once a BF7 marker is detected. Accordingly, a first test for BF7 is followed by a subsequent test for BF7 or another marker at a future date, including a subsequent screening or analysis of BF7 markers to establish a protocol for diagnosis, including imaging, or treatment including biopsy or other surgical (i.e. resection) or chemical (chemo or immunotherapy) treatment, optimally including a prescribed time interval for future diagnosis or treatment. A preferred protocol for using the BF7 assay or mAb BF7 preferably includes a first screening using the BF7 assay followed by additional testing to monitor expression of BF7 markers, optionally including another marker, at prescribed time intervals to determine progress or stages from an early stage of cancer, the risk of developing cancer beyond the stage assessed at the first screening or predicting the progression of the disease beyond any prior analysis of mAb BF7. Accordingly, the outcome of the BF7 assay on a patient sample may lead a clinician to recommend to the patient to continue the same treatment, or change treatment, to receive confirmatory diagnosis of disease progression through the use of further imaging procedures, or pursue active surveillance at scheduled intervals of time

In the clinical oncology laboratory today single markers (CA15-3, CEA, CYFRA, NSE etc.) are most commonly measured. However diagnostic assays with multiple biomarkers are being developed as they tend to provide higher sensitivity and specificity than a single marker assay. Such assays may comprise measurement of biomarker proteins and autoantibodies (e.g. Videssa Breast; Henderson, 2016), or measurement of methylation of DNA by PCR and hemoglobin by immunoassay (e.g. Cologuard; Imperiale, 2014), or measurement of HE4 (human epididymis protein) and CA125 combined with menopausal status into a numerical score called the risk of malignancy for ovarian cancer (ROMA; Bast, 2012), or microarray analysis of multigene signatures combined to a breast cancer risk score (Oncotype Dx; Paik, 2004). Likewise, it may be found advantageous to adapt the BF7 assay into incorporating the measurement of some BF7 marker species as polypeptides, together with some marker species as polynucleotides, using techniques known in the art for accurate identification of a polypeptide as well as techniques known in the art for accurate identification of a polynucleotide.

The present invention teaches about detection of BF7 markers in tissues, as well as in biological fluids, both in serum and in urine, from a subject in relation to cancer through immunoassay detection. Most conventional diagnostic assays, whether in oncology or other areas, are immunoassays. Indeed immunoassays have significant advantages over alternate methodologies insofar they are serum-based and cost-effective. Additionally, due to their versatility, they come in many test formats, i.e large automated immunoassay platforms and semi-automated smaller formats, and thus specifically tailored to the large and small clinical lab infrastructure settings, respectively. Other immunoassay formats are the commonly known home-based immunoassays, such as urine-based hormone dipstick assays. In a preferred embodiment cancer recurrence may be monitored with patient-friendly home-based assays in urine. At the other end of the spectrum are more sophisticated immunoassays based on nanofludics developments which render immunoassays even more portable and more sensitive, and well suited to point of care testing. In another embodiment of the present invention, rapid point of care cancer testing or therapy or recurrence monitoring is contemplated with the use of hand-held devices.

This invention encompasses all immunoassay formats. Indeed as a way of enabling assay multiplexing, planar solid supports like antibody arrays (Haab, 2006) or bead-based assays, such as but not limited to Luminex technology (Zeh, 2005) can be preferably used to simultaneously measure hundreds of analytes in a biological sample.

It is contemplated in the present invention that data analysis as described herein (Example 10) be improved with the use of further statistical methods, such as but not limited to logistic regression analysis, forest model analysis, and other statistical model that enables the integration of multiple analyte data with patient data (age, gender, stage, ethnicity, smoking status, family and personal history of cancer etc.) to generate a cutoff or a score, based on a robust algorithm, representing a likelihood of having cancer, of disease progression, of being cancer free, or needing further practitioner visit.

In a preferred embodiment the compositions of the invention are applied to small hand-held devices offering rapid immunoassay testing at point of care such as regional community centers or general practitioner's office, preferably in remote areas where patients have limited access to clinical centers and oncologist practices. However a general practitioner, or physician assistant, or skilled nurse may assist a patient in conducting the test of the present invention as well as reading and interpreting test results.

In a further preferred embodiment to ensure the assay of the present invention is made accessible to everyone regardless of the quality of care the subject may access or afford, a software interface is linked to the test to facilitate reading of the results. In some embodiments, a computer-based analysis program is used to translate the raw data generated by the detection assay (e.g. elevation, decrease of a given marker with respect to a reference value) into data of predictive value for a clinician, enabling the latter to make a better clinical assessment of the patient and optimize the care of the subject. The clinician can access the predictive data using any suitable means. Thus, in some preferred embodiments, the present invention provides the benefit that the clinician, or a general practitioner, a physician assistant, a skilled nurse, or the patient is presented with information derived from the raw data in its most useful form, such as for example, in the form of an application translating test results into simple visual instructions as well as recommendations for patient care such as “negative”, “surveillance”, “further testing required”, “follow-up required with doctor”, etc.

The invention also includes compositions of matter such as assay devices reaction mixtures, and systems for biological fluid analysis that enable quantitative detection of the markers, individually or collectively, together with related species, and either alone or in combination with other markers, to assess the health or condition of a patient. The test can be in a panel format including the combination of polypeptides and portions thereof, the polynucleotide, antibodies, or other entities or constructs described herein.

1. A method to diagnose colorectal cancer comprising:
reacting a human serum or urine sample with a non-human monoclonal antibody, wherein the antibody is immunologically reactive with a motif comprising an artificial 12-mer mimotope having at least 67% homology with HSFTTYLTVHPS;
quantifying a total amount of proteins in the serum or urine sample that are immunologically bound to the non-human monoclonal antibody, wherein the serum or urine proteins are comprised of a plurality of Sterile alpha motif domain-containing protein 14 (SAMD14, SEQ ID NO: 1), Nuclear pore complex protein Nup107 (NUP107, SEQ ID NO: 2), Putative IQ motif and ankyrin repeat domain-containing protein LOC642574 (LOC642574, SEQ ID NO: 6), Transcription initiation factor TFIID subunit 4 (TAF4, SEQ ID NO: 9), and Protein Bassoon (BSN, SEQ ID NO: 20) and combinations thereof;
comparing the total quantity of proteins measured in the quantifying step to a reference value; and
diagnosing a presence or absence of colorectal cancer with at least 70% sensitivity and at least 70% specificity based on a difference between the quantity of total proteins and the reference value.

2. The method of claim 1, wherein the non-human antibody is also immunologically reactive with a motif comprising an artificial 12-mer mimotope having at least 67% homology with TYYPSSIPGFTL and wherein the quantifying step is further comprised of quantitatively measuring a protein selected from the group consisting of Protein Probable rRNA-processing protein EBP2 (EBNA1BP2, SEQ ID NO: 3), FH1/FH2 domain-containing protein 1 (FHOD1, SEQ ID NO: 5), UPF0488 protein C8orf33 (C8orf33, SEQ ID NO: 7), 28S ribosomal protein S11 mitochondrial (MRPS11, SEQ ID NO: 8), Serrate RNA effector molecule homolog (SRRT, SEQ ID NO: 10), 40S ribosomal protein S13 (RPS13, SEQ ID NO: 11), 60S ribosomal protein L4 (RPL4, SEQ ID NO: 12), Voltage-gated potassium channel subunit beta-3 (KCNAB3, SEQ ID NO: 13), and Kelch-like protein 14 (KLHL14, SEQ ID NO: 15), and combinations thereof.

3. The system of claim 2, wherein the non-human antibody is also immunologically reactive with a motif comprising an artificial 12-mer mimotope having at least 67% homology with HLTHSPIPVRAM and wherein the quantifying step is further comprised of quantitatively measuring a protein selected from the group consisting of Transcriptional activator MN1 (MN1, SEQ ID NO: 4), Plakophilin-3 (PKP3, SEQ ID NO: 14), Ras-related protein Rab-27B (RAB27B, SEQ ID NO: 16), 6-phosphogluconolactonase (PGLS, SEQ ID NO: 17), Cyclin L2 (CCNL2, SEQ ID NO: 18), Iron-sulfur protein NUBPL (NUBPL, SEQ ID NO: 19), Histone H1.2 (HIST1H1C, SEQ ID NO: 21), Cytosolic Fe—S cluster assembly factor (NUBP2, SEQ ID NO: 23), Rho-related GTP-binding protein RhoU (RHOU, SEQ ID NO: 24), Collagen alpha-1 (I) chain (COL1A1, SEQ ID NO: 25), Tetraspanin (TSPAN7, SEQ ID NO: 27), and Ras-related protein RAB-11B (RAB11B, SEQ ID NO: 28), and combinations thereof.

4. The method of claim 1, wherein the human serum or urine sample is comprised of a first sample taken at a first time and a second sample from the patient taken at a second time and the quantifying step is comprised of a comparison of the total quantity of proteins in the serum or urine sample immunologically bound by the non-human antibody at the first time and the serum or urine proteins bound by the non-human antibody at the second time.

5. The method of claim 4, further comprising the step of administering to the patient active surveillance based on the diagnosis.

6. The method of claim 4, further comprising the step of assessing the efficacy of a treatment for colorectal cancer based on the measurement of the of total quantity of the proteins.

7. The method of claim 6, wherein the assessment of the efficacy of the colorectal cancer treatment precedes metastasis.

8. A method to diagnose colorectal cancer comprising:
separating a collection of proteins present in a human serum or urine sample using a reaction mixture comprising a non-human monoclonal antibody that is immunologically reactive with a motif comprising a 12-mer mimotope having at least 67% homology with HSFTTYLTVHPS and is also immunologically reactive with a plurality of proteins selected from the group consisting of Sterile alpha motif domain-containing protein 14 (SAMD14, SEQ ID NO: 1), Nuclear pore complex protein Nup107 (NUP107, SEQ ID NO: 2), Putative IQ motif and ankyrin repeat domain-containing protein LOC642574 (LOC642574, SEQ ID NO: 6), Transcription initiation factor TFIID subunit 4 (TAF4, SEQ ID NO: 9), and Protein Bassoon (BSN, SEQ ID NO: 20);
isolating a collective quantity of the plurality of proteins present in the human serum or urine sample by immunologically binding the plurality of proteins to a solid support comprising the non-human monoclonal antibody immunologically bound to the plurality of biomarkers present in the human serum or urine sample;
quantifying the collective quantity of the plurality of proteins immunologically bound to the solid support;
comparing the quantified collective amount with a reference value; and
diagnosing a presence or absence of colorectal cancer with at least 70% sensitivity and at least 70% specificity based on a difference between the collective quantity of the plurality of proteins and the reference value.

9. The method of claim 8, wherein the non-human antibody is also immunologically reactive with a motif comprising a 12-mer mimotope having at least 67% homology with TYYPSSIPGFTL and wherein the plurality of proteins present in the human or urine sample is further comprised of probable rRNA-processing protein EBP2 (EBNA1BP2, SEQ ID NO: 3), FH1/FH2 domain-containing protein 1 (FHOD1, SEQ ID NO: 5), UPF0488 protein C8orf33 (C8orf33, SEQ ID NO: 7), 28S ribosomal protein S11 mitochondrial (MRPS11, SEQ ID NO: 8), Serrate RNA effector molecule homolog (SRRT, SEQ ID NO: 10), 40S ribosomal protein S13 (RPS13, SEQ ID NO: 11), 60S ribosomal protein L4 (RPL4, SEQ ID NO: 12), Voltage-gated potassium channel subunit beta-3 (KCNAB3, SEQ ID NO: 13), and Kelch-like protein 14 (KLHL14, SEQ ID NO: 15), and combinations thereof.

10. The method of claim 8, wherein the non-human antibody is also immunologically reactive with 12-mer mimotope having at least 67% homology with HLTHSPIPVRAM, and
wherein the plurality of proteins present in the human or urine sample is further comprised of Transcriptional activator MN1 (MN1, SEQ ID NO: 4), Plakophilin-3 (PKP3, SEQ ID NO: 14), Ras-related protein Rab-27B (RAB27B, SEQ ID NO: 16), 6-phosphogluconolactonase (PGLS, SEQ ID NO: 17), Cyclin L2 (CCNL2, SEQ ID NO: 18), Iron-sulfur protein NUBPL (NUBPL, SEQ ID NO: 19), Histone H1.2 (HISTIHIC, SEQ ID NO: 21), Cytosolic Fe—S cluster assembly factor (NUBP2, SEQ ID NO: 23), Rho-related GTP-binding protein RhoU (RHOU, SEQ ID NO: 24), Collagen alpha-1 (I) chain (COL1A1, SEQ ID NO: 25), Tetraspanin (TSPAN7, SEQ ID NO: 27), and Ras-related protein RAB-11B (RAB11B, SEQ ID NO: 28), and combinations thereof.

11. The method of claim 8, wherein the plurality of biomarkers present in the human or urine sample is further comprised of Ubiquilin 4 (UBQLN4, SEQ ID NO: 22), Galectin-3 binding protein (LGALS3BP, SEQ ID NO: 26). and combinations thereof.

12. The method of claim 8, further comprising the step of detecting a separate biomarker selected from the group consisting of Iron-sulfur protein NUBPL, (NUBPL), cyclin L2 (CLL2), cytosolic Fe—S cluster assembly factor (NUBP2), Ki-67 (Ki-67), P16INK4a (p16), Estrogen receptor (ER-alfa), Progesterone receptor (PR), c-erbB-2 (HER-2), soluble HER2, Cathepsin D, CA15-3 (CA15-3), CA27.29 (CA27.29), Carcinoma embryonic antigen (CEA), Vimentin (Vimentin), Prostate specific antigen (PSA), Prostatic acid phosphatase (PAP), Kallikrein-2 (KLK-2), p504S (p504S), Tumor Protein p63 (p63), Chromogranin A (CgA), Progastrin releasing peptide type 3 (ProGRP), Neuron specific enolase (NSE), Melanocyte lineage-specific antigen (Gp100), MART-1 (MART-1), MAGE-1 (MAGE-1), Calcium binding protein A4/Metastasin 100 (S100A4), Alfa-fetoprotein (AFP), Macrophage inhibitory cytokine (MIC-1), Osteopontin (OSPN), CA19-9 (CA19-9), Mucin-16/ovarian carcinoma antigen CA-125 (CA-125), Leukocyte common antigen (CD45 LCA), CD68 (CD68), Cytokeratins 5, 6 (CK5/6), Cytokeratin 16, 17 and 18 (CK16/17/18), Cytokeratin 17 (CK17), Cytokeratin 19 fragment/CYFRA 21.1, B-cell lymphoma-2 (BCL-2), B-Lymphocyte antigen (CD20), Hematopoietic progenitor CD34 (CD34), Proto-oncogene P53 (p53), Mucin-2 (MUC-2), Mucin-3A (MUC-3), Mucin-4 (MUC-4), Mucin 5AC (MUC-5AC), Mucin-6 (MUC-6), Proliferating cell nuclear antigen (PCNA), Tyrosinase (Tyr), Prostate specific membrane antigen (PSMA-1), Calcium binding protein (S1002), Tissue inhibitor of metalloproteinase (TIMP-1), Squamous cell carcinoma antigen (SCC), Androgen Receptor (ARC), Urokinase plasminogen activator (UPA), by the non-human antibody at the second Plasminogen activator inhibitor (PAI), and Protein uncharacterized ENSP0381381, CA-242, CYFRA21-1.

13. The method of claim 9, wherein the measurement of the separate biomarker is performed following a first and a second measurement of the plurality of proteins of claim 8 and a comparison of each of the first and second measurement with a reference value, and the measurement of the separate biomarker is correlated to a progression of colorectal cancer.

14. A method to diagnosed colorectal cancer comprising,
a) reacting a human serum or urine sample with a non-human monoclonal capture antibody fixed on a solid support to immunologically bind a plurality of proteins selected from the group consisting of Sterile alpha motif domain-containing protein 14 (SAMD14, SEQ ID NO: 1), Nuclear pore complex protein Nup107 (NUP107, SEQ ID NO: 2), Putative IQ motif and ankyrin repeat domain-containing protein LOC642574 (LOC642574, SEQ ID NO: 6); Transcription initiation factor TFIID subunit 4 (TAF4, SEQ ID NO: 9); Protein Bassoon (BSN, SEQ ID NO: 20) and combinations thereof;
wherein the non-human monoclonal capture antibody also immunologically binds a plurality of proteins present in the human or urine sample selected from the group consisting of Probable rRNA-processing protein EBP2 (EBNA1BP2, SEQ ID NO: 3), FH1/FH2 domain-containing protein 1 (FHOD1, SEQ ID NO: 5), UPF0488 protein C8orf33 (C8orf33, SEQ ID NO: 7), 28S ribosomal protein S11 mitochondrial (MRPS11, SEQ ID NO: 8); Serrate RNA effector molecule homolog (SRRT, SEQ ID NO: 10), 40S ribosomal protein S13 (RPS13, SEQ ID NO: 11), 60S ribosomal protein L4 (RPL4, SEQ ID NO: 12), Voltage-gated potassium channel subunit beta-3 (KCNAB3, SEQ ID NO: 13), Kelch-like protein 14 (KLHL14, SEQ ID NO: 15); and combinations thereof; and
wherein the non-human monoclonal capture antibody also immunologically binds a plurality of proteins present in the human or urine sample selected from the group consisting of Transcriptional activator MN1 (MN1, SEQ ID NO: 4); Plakophilin-3 (PKP3, SEQ ID NO: 14), Ras-related protein Rab-27B (RAB27B, SEQ ID NO: 16), 6-phosphogluconolactonase (PGLS, SEQ ID NO: 17), Cyclin L2 (CCNL2, SEQ ID NO: 18), Iron-sulfur protein NUBPL (NUBPL, SEQ ID NO: 19), Histone H1.2 (HIST1H1C, SEQ ID NO: 21), Cytosolic Fe—S cluster assembly factor (NUBP2, SEQ ID NO: 23), Rho-related GTP-binding protein RhoU (RHOU, SEQ ID NO: 24), Collagen alpha-1 (I) chain (COL1A1, SEQ ID NO: 25), Tetraspanin (TSPAN7, SEQ ID NO: 27), Ras-related protein RAB-11B (RAB11B, SEQ ID NO: 28), and combinations thereof;
b) labelling a combination of the capture antibody and the immunologically bound proteins with a detection antibody immunologically bound to the combination of the capture antibody and the immunologically bound proteins from the serum or urine sample, wherein the detection antibody is further comprised of a detectable moiety, and
c) measuring a quantity of the plurality of immunologically bound proteins using the detection moiety;
d) comparing the measured quantity of the immunologically bound proteins with a reference value; and
e) diagnosing a presence or absence of colorectal cancer with at least 70% sensitivity and at least 70% specificity based on a difference between the measured quantity of the plurality of immunologically bound proteins and the reference value.

15. A method for detection of colorectal cancer at an early stage comprising:
a) generating a quantitative measurement of a total amount of a plurality of proteins present in a human serum or urine sample, wherein the naturally expressed human proteins are immunologically bound to a non-human monoclonal antibody that is immunologically reactive with an artificial mimotope having 67% homology with a 12-mer HSFTTYLTVHPS, wherein the non-human monoclonal antibody immunologically binds a plurality of biomarkers present in the serum or urine sample selected from the group consisting of Sterile alpha motif domain-containing protein 14 (SAMD14, SEQ ID NO: 1), Nuclear pore complex protein Nup107 (NUP107, SEQ ID NO: 2), Putative IQ motif and ankyrin repeat domain-containing protein LOC642574 (LOC642574, SEQ ID NO: 6), Transcription initiation factor TFIID subunit 4 (TAF4, SEQ ID NO: 9), Protein Bassoon (BSN, SEQ ID NO: 20) and combinations thereof;
b) measuring a total quantity of the immunologically bound proteins bound to the non-human antibody to generate a quantitative value of a summation of the total quantity of immunologically bound proteins;
c) comparing the quantitative value of the total quantity bound proteins with a quantitative reference value; and
d) diagnosing a presence or absence of breast cancer with at least 70% sensitivity and at least 70% specificity based on a difference between the quantity of total proteins and the reference value.

16. A method to diagnose colorectal cancer comprising:
a) reacting a human serum or urine sample a non-human monoclonal antibody that is immunologically reactive with:
1) an artificial mimotope having at least 67% homology with a 12-mer HSFTTYLTVHPS, and
2) a motif shared by a collection of proteins comprising Sterile alpha motif domain-containing protein 14 (SAMD14, SEQ ID NO: 1); Nuclear pore complex protein Nup107 (NUP107, SEQ ID NO: 2); Putative IQ motif and ankyrin repeat domain-containing protein LOC642574 (LOC642574, SEQ ID NO: 6); Transcription initiation factor TFIID subunit 4 (TAF4, SEQ ID NO: 9); Protein Bassoon (BSN, SEQ ID NO: 20;
b) measuring a collective quantity of proteins having the shared motif and immunologically bound to the non-human monoclonal antibody in the human serum or urine sample;
c) comparing the collective quantity of bound proteins with a reference value; and
d) diagnosing a presence or absence of breast cancer with at least 70% sensitivity and at least 70% specificity based on a difference between the quantity of total bound proteins and the reference value.

17. The method of claim 16, wherein the motif is also shared by Probable rRNA-processing protein EBP2 (EBNA1BP2, SEQ ID NO: 3); FH1/FH2 domain-containing protein 1 (FHOD1, SEQ ID NO: 5); UPF0488 protein C8orf33 (C8orf33, SEQ ID NO: 7); 28S ribosomal protein S11, mitochondrial (MRPS11, SEQ ID NO: 8); Serrate RNA effector molecule homolog (SRRT, SEQ ID NO: 10); 40S ribosomal protein S13 (RPS13, SEQ ID NO: 11); 60S ribosomal protein L4 (RPL4, SEQ ID NO: 12); Voltage-gated potassium channel subunit beta-3 (KCNAB3, SEQ ID NO: 13); Kelch-like protein 14 (KLHL14, SEQ ID NO: 15), and combinations thereof.

18. The method of claim 16, wherein the motif is also shared by Transcriptional activator MN1 (MN1, SEQ ID NO: 4); Plakophilin-3 (PKP3, SEQ ID NO: 14), Ras-related protein Rab-27B (RAB27B, SEQ ID NO: 16), 6-phosphogluconolactonase (PGLS, SEQ ID NO: 17), Cyclin L2 (CCNL2, SEQ ID NO: 18), Iron-sulfur protein NUBPL (NUBPL, SEQ ID NO: 19), Histone H1.2 (HIST1H1C, SEQ ID NO: 21), Cytosolic Fe—S cluster assembly factor (NUBP2, SEQ ID NO: 23), Rho-related GTP-binding protein RhoU (RHOU, SEQ ID NO: 24), Collagen alpha-1 (I) chain (COL1A1, SEQ ID NO: 25), Tetraspanin (TSPAN7, SEQ ID NO: 27), Ras-related protein RAB-11B (RAB11B, SEQ ID NO: 28), and combinations thereof.


SEQUENCE LISTING (31 sequences)

SEQ ID NO: 1 - protein, 417 aa, Homo sapiens
MASSKLREPVDEVFDLDLAVPETARLDSSLHKARAQLLAKGRRHRPSRSRLRDSASSAEDGEGSDGPGGKVTDGCGSPLHRLRSPLHSGPGSPAGGSFCLDPPGLRRSLDEDEPPPSPLTRYRPLHNAASHEGLAAASCSPPRSAPSSDSSPSFVRRHPRAEPHSEDDSRDASPPEPASPTIGLDKKTRRKFLDLGVTLRRASTGKSRKEKGSNRLSMGSRESVEGSGRSGGSPFLPFSWFTDSGKGSASSGSTTSPTCSPKHEGFSPKKSASQESTLSDDSTPPSSSPKIPSGPWQEAKCSYPYHTLSQSSDEFLDEPLPPVHHWTSQQVGQWLQSLNLEQYAAEFAARQVDGPQLLQLDGSKLKSLGLSNSHDRALVKRKLKEMAAAAEKERKAQEKAARQREKLRRREQEAKKS

SEQ ID NO: 2 - protein, 925 aa, Homo sapiens
MDRSGFGEISSPVIREAEVTRTARKQSAQKRVLLQASQDENFGNTTPRNQVIPRTPSSFRQPFTPTSRSLLRQPDISCILGTGGKSPRLTQSSGFFGNLSMVTNLDDSNWAAAFSSQRSGLFTNTEPHSITEDVTISAVMLREDDPGEAASMSMFSDFLQSFLKHSSSTVFDLVEEYENICGSQVNILSKIVSRATPGLQKFSKTASMLWLLQQEMVTWRLLASLYRDRIQSALEEESVFAVTAVNASEKTVVEALFQRDSLVRQSQLVVDWLESIAKDEIGEFSDNIEFYAKSVYWENTLHTLKQRQLTSYVGSVRPLVTELDPDAPIRQKMPLDDLDREDEVRLLKYLFTLIRAGMTEEAQRLCKRCGQAWRAATLEGWKLYHDPNVNGGTELEPVEGNPYRRIWKISCWRMAEDELFNRYERAIYAALSGNLKQLLPVCDTWEDTVWAYFRVMVDSLVEQEIQTSVATLDETEELPREYLGANWTLEKVFEELQATDKKRVLEENQEHYHIVQKFLILGDIDGLMDEFSKWLSKSRNNLPGHLLRFMTHLILFFRTLGLQTKEEVSIEVLKTYIQLLIREKHTNLIAFYTCHLPQDLAVAQYALFLESVTEFEQRHHCLELAKEADLDVATITKTVVENIRKKDNGEFSHHDLAPALDTGTTEEDRLKIDVIDWLVFDPAQRAEALKQGNAIMRKFLASKKHEAAKEVFVKIPQDSIAEIYNQCEEQGMESPLPAEDDNAIREHLCIRAYLEAHETFNEWFKHMNSVPQKPALIPQPTFTEKVAHEHKEKKYEMDFGIWKGHLDALTADVKEKMYNVLLFVDGGWMVDVREDAKEDHERTHQMVLLRKLCLPMLCFLLHTILHSTGQYQECLQLADMVSSERHKLYLVFSKEELRKLLQKLRESSLMLLDQGLDPLGYEIQL

SEQ ID NO: 3 - protein, 306 aa, Homo sapiens
MDTPPLSDSESESDESLVTDRELQDAFSRGLLKPGLNVVLEGPKKAVNDVNGLKQCLAEFKRDLEWVERLDVTLGPVPEIGGSEAPAPQNKDQKAVDPEDDFQREMSFYRQAQAAVLAVLPRLHQLKVPTKRPTDYFAEMAKSDLQMQKIRQKLQTKQAAMERSEKAKQLRALRKYGKKVQTEVLQKRQQEKAHMMNAIKKYQKGFSDKLDFLEGDQKPLAQRKKAGAKGQQMRKGPSAKRRYKNQKFGFGGKKKGSKWNTRESYDDVSSFRAKTAHGRGLKRPGKKGSNKRPGKRTREKMKNRTH

SEQ ID NO: 4 - protein, 1320 aa, Homo sapiens
MFGLDQFEPQVNSRNAGQGERNFNETGLSMNTHFKAPAFHTGGPPGPVDPAMSALGEPPILGMNMEPYGFHARGHSELHAGGLQAQPVHGFFGGQQPHHGHPGSHHPHQHHPHFGGNFGGPDPGASCLHGGRLLGYGGAAGGLGSQPPFAEGYEHMAESQGPESFGPQRPGNLPDFHSSGASSHAVPAPCLPLDQSPNRAASFHGLPSSSGSDSHSLEPRRVTNQGAVDSLEYNYPGEAPSGHFDMFSPSDSEGQLPHYAAGRQVPGGAFPGASAMPRAAGMVGLSKMHAQPPQQQPQQQQQPQQQQQQHGVFFERFSGARKMPVGLEPSVGSRHPLMQPPQQAPPPPQQQPPQQPPQQQPPPPPGLLVRQNSCPPALPRPQQGEAGTPSGGLQDGGPMLPSQHAQFEYPIHRLENRSMHPYSEPVFSMQHPPPQQAPNQRLQHFDAPPYMNVAKRPRFDFPGSAGVDRCASWNGSMHNGALDNHLSPSAYPGLPGEFTPPVPDSFPSGPPLQHPAPDHQSLQQQQQQQQQQQQQQQQQQQQQQQQQQQQRQNAALMIKQMASRNQQQRLRQPNLAQLGHPGDVGQGGLVHGGPVGGLAQPNFEREGGSTGAGRLGTFEQQAPHLAQESAWFSGPHPPPGDLLPRRMGGSGLPADCGPHDPSLAPPPPPGGSGVLFRGPLQEPMRMPGEGHVPALPSPGLQFGGSLGGLGQLQSPGAGVGLPSAASERRPPPPDFATSALGGQPGFPFGAAGRQSTPHSGPGVNSPPSAGGGGGSSGGGGGGGAYPPQPDFQPSQRTSASKLGALSLGSFNKPSSKDNLFGQSCLAALSTACQNMIASLGAPNLNVTFNKKNPPEGKRKLSQNETDGAAVAGNPGSDYFPGGTAPGAPGPGGPSGTSSSGSKASGPPNPPAQGDGTSLSPNYTLESTSGNDGKPVSGGGGRGRGRRKRDSGHVSPGTFFDKYSAAPDSGGAPGVSPGQQQASGAAVGGSSAGETRGAPTPHEKALTSPSWGKGAELLLGDQPDLIGSLDGGAKSDSSSPNVGEFASDEVSTSYANEDEVSSSSDNPQALVKASRSPLVTGSPKLPPRGVGAGEHGPKAPPPALGLGIMSNSTSTPDSYGGGGGPGHPGTPGLEQVRTPTSSSGAPPPDEIHPLEILQAQIQLQRQQFSISEDQPLGLKGGKKGECAVGASGAQNGDSELGSCCSEAVKSAMSTIDLDSLMAEHSAAWYMPADKALVDSADDDKTLAPWEKAKPQNPNSKEAHDLPANKASASQPGSHLQCLSVHCTDDVGDAKARASVPTWRSLHSDISNRFGTFVAALT

SEQ ID NO: 5 - protein, 1164 aa, Homo sapiens
MAGGEDRGDGEPVSVVTVRVQYLEDTDPFACANFPEPRRAPTCSLDGALPLGAQIPAVHRLLGAPLKLEDCALQVSPSGYYLDTELSLEEQREMLEGFYEEISKGRKPTLILRTQLSVRVNAILEKLYSSSGPELRRSLFSLKQIFQEDKDLVPEFVHSEGLSCLIRVGAAADHNYQSYILRALGQLMLFVDGMLGVVAHSDTIQWLYTLCASLSRLVVKTALKLLLVFVEYSENNAPLFIRAVNSVASTTGAPPWANLVSILEEKNGADPELLVYTVTLINKTLAALPDQDSFYDVTDALEQQGMEALVQRHLGTAGTDVDLRTQLVLYENALKLEDGDIEEAPGAGGRRERRKPSSEEGKRSRRSLEGGGCPARAPEPGPTGPASPVGPTSSTGPALLTGPASSPVGPPSGLQASVNLFPTISVAPSADTSSERSIYKARFLENVAAAETEKQVALAQGRAETLAGAMPNEAGGHPDARQLWDSPETAPAARTPQSPAPCVLLRAQRSLAPEPKEPLIPASPKAEPIWELPTRAPRLSIGDLDFSDLGEDEDQDMLNVESVEAGKDIPAPSPPLPLLSGVPPPPPLPPPPPIKGPFPPPPPLPLAAPLPHSVPDSSALPTKRKTVKLFWRELKLAGGHGVSASRFGPCATLWASLDPVSVDTARLEHLFESRAKEVLPSKKAGEGRRTMTTVLDPKRSNAINIGLTTLPPVHVIKAALLNFDEFAVSKDGIEKLLTMMPTEEERQKIEEAQLANPDIPLGPAENFLMTLASIGGLAARLQLWAFKLDYDSMEREIAEPLFDLKVGMEQLVQNATFRCILATLLAVGNFLNGSQSSGFELSYLEKVSEVKDTVRRQSLLHHLCSLVLQTRPESSDLYSEIPALTRCAKVDFEQLTENLGQLERRSRAAEESLRSLAKHELAPALRARLTHFLDQCARRVAMLRIVHRRVCNRFHAFLLYLGYTPQAAREVRIMQFCHTLREFALEYRTCRERVLQQQQKQATYRERNKTRGRMITETEKFSGVAGEAPSNPSVPVAVSSGPGRGDADSHASMKSLLTSRPEDTTHNRRSRGMVQSSSPIMPTVGPSTASPEEPPGSSLPSDTSDEIMDLLVQSVTKSSPRALAARERKRSRGNRKSLRRTLKSGLGDDLVQALGLSKGPGLEV

SEQ ID NO: 6 - protein, 604 aa, Homo sapiens
MQEGPSAFREGCPAGDSTPSSPFLGAPELRARRGSQDPRRRGAGEVPADLGPLRVPGSRAASSCGLCGREPRLGRAGGFESRPAEDRAARAIQGAFRQLRARRELARRREERREYLEQMETPQKEAYLAPVRREQEAARRLREQEEAAQRERREELQRRRRLLDAAFDGDVGEIRAVLKEVEQLLTREGVGHDEAGEARRLQRRVALAECEDSYGNTPLSEAAAGGQPLAIQLRAELGASPNSKGAFGPTPLYRAAFGGHLAAVEVLLKLGADPRVYAEDGSTPERVASLDTVVSVLRSWDLSLTEAMLQNMEAEQQRRAQEAQRHKEAEAERMTLKVQQLTREQQQCHKELQQAYCELSRRISEHDQCEWRCMDKTKLTLQAIKDTEAQVDRLRQEAQKAEEALAMARLELREQTQEGEEEAPGLKCQVTELHDVLMKDVGNRIRADGRSVLRARRGRFGVWTDSSDLMGVPRWPLVIDPLGQAATFLRYQDTNYVDTVNPEPLRPETMWLALLGALRYGKPLVFDLREEDLFPVVQRQLEAVQERYLSLLRPTDGPEYSPTQFQEQRLEHFRLFFVTKVQWPPAEQLQVLLPVRVQLPGTGL

SEQ ID NO: 7 - protein, 229 aa, Homo sapiens
MAALGHLAGEAAAAPGPGTPCASRGARLPGPVSSARNPSTVCLCPEQPTCSNADSRAHPLGDEGGTASKKQKNKKKTRNRASVANGGEKASEKLAPEEVPLSAEAQAQQLAQELAWCVEQLELGLKRQKPTPKQKEQAIGAIRTLRSKRTPLPRKRQLMHSLFGDYRAQMEAEWREALRALRAAAYSAQVQPVDGATRKKSQRVCRPRSIWRAKATLDMPDEEFRFNFF

SEQ ID NO: 8 - protein, 194 aa, Homo sapiens
MQAVRNAGSRFLRSWTWPQTAGRVVARTPAGTICTGARQLQDAAAKQKVEQNAAPSHTKFSIYPPIPGEESSLRWAGKKFEEIPIAHIKASHNNTQIQVVSASNEPLAFASCGTEGFRNAKKGTGIAAQTAGIAAAARAKQKGVIHIRVVVKGLGPGRLSAMHGLIMGGLEVISITDNTPIPHNGCRPRKARKL

SEQ ID NO: 9 - protein, 1085 aa, Homo sapiens
MAAGSDLLDEVFFNSEVDEKVVSDLVGSLESQLAASAAHHHHLAPRTPEVRAAAAGALGNHVVSGSPAGAAGAGPAAPAEGAPGAAPEPPPAGRARPGGGGPQRPGPPSPRRPLVPAGPAPPAAKLRPPPEGSAGSCAPVPAAAAVAAGPEPAPAGPAKPAGPAALAARAGPGPGPGPGPGPGPGPGKPAGPGAAQTLNGSAALLNSHHAAAPAVSLVNNGPAALLPLPKPAAPGTVIQTPPFVGAAAPPAPAAPSPPAAPAPAAPAAAPPPPPPAPATLARPPGHPAGPPTAAPAVPPPAAAQNGGSAGAAPAPAPAAGGPAGVSGQPGPGAAAAAPAPGVKAESPKRVVQAAPPAAQTLAASGPASTAASMVIGPTMQGALPSPAAVPPPAPGTPTGLPKGAAGAVTQSLSRTPTATTSGIRATLTPTVLAPRLPQPPQNPTNIQNFQLPPGMVLVRSENGQLLMIPQQALAQMQAQAHAQPQTTMAPRPATPTSAPPVQISTVQAPGTPIIARQVTPTTIIKQVSQAQTTVQPSATLQRSPGVQPQLVLGGAAQTASLGTATAVQTGTPQRTVPGATTTSSAATETMENVKKCKNFLSTLIKLASSGKQSTETAANVKELVQNLLDGKIEAEDFTSRLYRELNSSPQPYLVPFLKRSLPALRQLTPDSAAFIQQSQQQPPPPTSQATTALTAVVLSSSVQRTAGKTAATVTSALQPPVLSLTQPTQVGVGKQGQPTPLVIQQPPKPGALIRPPQVTLTQTPMVALRQPHNRIMLTTPQQIQLNPLQPVPVVKPAVLPGTKALSAVSAQAAAAQKNKLKEPGGGSFRDDDDINDVASMAGVNLSEESARILATNSELVGTLTRSCKDETFLLQAPLQRRILEIGKKHGITELHPDVVSYVSHATQQRLQNLVEKISETAQQKNFSYKDDDRYEQASDVRAQLKFFEQLDQIEKQRKDEQEREILMRAAKSRSRQEDPEQLRLKQKAKEMQQQELAQMRQRDANLTALAAIGPRKKRKVDCPGPGSGAEGSGPGSVVPGSSGVGTPRQFTRQRITRVNLRDLIFCLENERETSHSLLLYKAFLK

SEQ ID NO: 10 - protein, 876 aa, Homo sapiens
MGDSDDEYDRRRRDKFRRERSDYDRSRERDERRRGDDWNDREWDRGRERRSRGEYRDYDRNRRERFSPPRHELSPPQKRMRRDWDEHSSDPYHSGYEMPYAGGGGGPTYGPPQPWGHPDVHIMQHHVLPIQARLGSIAEIDLGVPPPVMKTFKEFLLSLDDSVDETEAVKRYNDYKLDFRRQQMQDFFLAHKDEEWFRSKYHPDEVGKRRQEARGALQNRLRVFLSLMETGWFDNLLLDIDKADAIVKMLDAAVIKMEGGTENDLRILEQEEEEEQAGKPGEPSKKEEGRAGAGLGDGERKTNDKDEKKEDGKQAENDSSNDDKTKKSEGDGDKEEKKEDSEKEAKKSSKKRNRKHSGDDSFDEGSVSESESESESGQAEEEKEEAEEALKEKEKPKEEEWEKPKDAAGLECKPRPLHKTCSLFMRNIAPNISRAEIISLCKRYPGFMRVALSEPQPERRFFRRGWVTFDRSVNIKEICWNLQNIRLRECELSPGVNRDLTRRVRNINGITQHKQIVRNDIKLAAKLIHTLDDRTQLWASEPGTPPLPTSLPSQNPILKNITDYLIEEVSAEEEELLGSSGGAPPEEPPKEGNPAEINVERDEKLIKVLDKLLLYLRIVHSLDYYNTCEYPNEDEMPNRCGIIHVRGPMPPNRISHGEVLEWQKTFEEKLTPLLSVRESLSEEEAQKMGRKDPEQEVEKFVTSNTQELGKDKWLCPLSGKKFKGPEFVRKHIFNKHAEKIEEVKKEVAFFNNFLTDAKRPALPEIKPAQPPGPAQILPPGLTPGLPYPHQTPQGLMPYGQPRPPILGYGAGAVRPAVPTGGPPYPHAPYGAGRGNYDAFRGQGGYPGKPRNRMVRGDPRAIVEYRDLDAPDDVDFF

SEQ ID NO: 11 - protein, 151 aa, Homo sapiens [sequence omitted]

SEQ ID NO: 12 - protein, 427 aa, Homo sapiens [sequence omitted]

SEQ ID NO: 13 - protein, 404 aa, Homo sapiens
MQVSIACTEQNLRSRSSEDRLCGPRPGPGGGNGGPAGGGHGNPPGGGGSGPKARAALVPRPPAPAGALRESTGRGTGMKYRNLGKSGLRVSCLGLGTWVTFGSQISDETAEDVLTVAYEHGVNLFDTAEVYAAGKAERTLGNILKSKGWRRSSYVITTKIFWGGQAETERGLSRKHIIEGLRGSLERLQLGYVDIVFANRSDPNCPMEEIVRAMTYVINQGLALYWGTSRWGAAEIMEAYSMARQFNLIPPVCEQAEHHLFQREKVEMQLPELYHKIGVGSVTWYPLACGLITSKYDGRVPDTCRASIKGYQWLKDKVQSEDGKKQQAKVMDLLPVAHQLGCTVAQLAIAWCLRSEGVSSVLLGVSSAEQLIEHLGALQVLSQLTPQTVMEIDGLLGNKPHSKK

SEQ ID NO: 14 - protein, 797 aa, Homo sapiens
MQDGNFLLSALQPEAGVCSLALPSDLQLDRRGAEGPEAERLRAARVQEQVRARLLQLGQQPRHNGAAEPEPEAETARGTSRGQYHTLQAGFSSRSQGLSGDKTSGFRPIAKPAYSPASWSSRSAVDLSCSRRLSSAHNGGSAFGAAGYGGAQPTPPMPTRPVSFHERGGVGSRADYDTLSLRSLRLGPGGLDDRYSLVSEQLEPAATSTYRAFAYERQASSSSSRAGGLDWPEATEVSPSRTIRAPAVRTLQRFQSSHRSRGVGGAVPGAVLEPVARAPSVRSLSLSLADSGHLPDVHGFNSYGSHRTLQRLSSGFDDIDLPSAVKYLMASDPNLQVLGAAYIQHKCYSDAAAKKQARSLQAVPRLVKLFNHANQEVQRHATGAMRNLIYDNADNKLALVEENGIFELLRTLREQDDELRKNVTGILWNLSSSDHLKDRLARDTLEQLTDLVLSPLSGAGGPPLIQQNASEAEIFYNATGFLRNLSSASQATRQKMRECHGLVDALVTSINHALDAGKCEDKSVENAVCVLRNLSYRLYDEMPPSALQRLEGRGRRDLAGAPPGEVVGCFTPQSRRLRELPLAADALTFAEVSKDPKGLEWLWSPQIVGLYNRLLQRCELNRHTTEAAAGALQNITAGDRRWAGVLSRLALEQERILNPLLDRVRTADHHQLRSLTGLIRNLSRNARNKDEMSTKVVSHLIEKLPGSVGEKSPPAEVLVNIIAVLNNLVVASPIAARDLLYFDGLRKLIFIKKKRDSPDSEKSSRAASSLLANLWQYNKLHRDFRAKGYRKEDFLGP

SEQ ID NO: 15 - protein, 628 aa, Homo sapiens
MSRSGDRTSTFDPSHSDNLLHGLNLLWRKQLFCDVTLTAQGQQFHCHKAVLASCSQYFRSLFSSHPPLGGGVGGQDGLGAPKDQQQPPQQQPSQQQQPPPQEEPGTPSSSPDDKLLTSPRAINNLVLQGCSSIGLRLVLEYLYTANVTLSLDTVEEVLSVSKILHIPQVTKLCVQFLNDQISVQNYKQVCKIAALHGLEETKKLANKYLVEDVLLLNFEEMRALLDSLPPPVESELALFQMSVLWLEHDRETRMQYAPDLMKRLRFALIPAPELVERVQSVDFMRTDPVCQKLLLDAMNYHLMPFRQHCRQSLASRIRSNKKMLLLVGGLPPGPDRLPSNLVQYYDDEKKTWKILTIMPYNSAHHCVVEVENFLFVLGGEDQWNPNGKHSTNFVSRYDPRFNSWIQLPPMQERRASFYACRLDKHLYVIGGRNETGYLSSVECYNLETNEWRYVSSLPQPLAAHAGAVHNGKIYISGGVHNGEYVPWLYCYDPVMDVWARKQDMNTKRAIHTLAVMNDRLYAIGGNHLKGFSHLDVMLVECYDPKGDQWNILQTPILEGRSGPGCAVLDDSIYLVGGYSWSMGAYKSSTICYCPEKGTWTELEGDVAEPLAGPACVTVILPSCVPYNK

SEQ ID NO: 16 - protein, 218 aa, Homo sapiens
MTDGDYDYLIKLLALGDSGVGKTTFLYRYTDNKFNPKFITTVGIDFREKRVVYNAQGPNGSSGKAFKVHLQLWDTAGQERFRSLTTAFFRDAMGFLLMFDLTSQQSFLNVRNWMSQLQANAYCENPDIVLIGNKADLPDQREVNERQARELADKYGIPYFETSAATGQNVEKAVETLLDLIMKRMEQCVEKTQIPDTVNGGNSGNLDGEKPPEKKCIC

SEQ ID NO: 17 - protein, 258 aa, Homo sapiens
MAAPAPGLISVFSSSQELGAALAQLVAQRAACCLAGARARFALGLSGGSLVSMLARELPAAVAPAGPASLARWTLGFCDERLVPFDHAESTYGLYRTHLLSRLPIPESQVITINPELPVEEAAEDYAKKLRQAFQGDSIPVFDLLILGVGPDGHTCSLFPDHPLLQEREKIVAPISDSPKPPPQRVTLTLPVLNAARTVIFVATGEGKAAVLKRILEDQEENPLPAALVQPHTGKLCWFLDEAAARLLTVPFEKHSTL

SEQ ID NO: 18 - protein, 520 aa, Homo sapiens
MAAAAAAAGAAGSAAPAAAAGAPGSGGAPSGSQGVLIGDRLYSGVLITLENCLLPDDKLRFTPSMSSGLDTDTETDLRVVGCELIQAAGILLRLPQVAMATGQVLFQRFFYTKSFVKHSMEHVSMACVHLASKIEEAPRRIRDVINVFHRLRQLRDKKKPVPLLLDQDYVNLKNQIIKAERRVLKELGFCVHVKHPHKIIVMYLQVLECERNQHLVQTSWNYMNDSLRTDVFVRFQPESIACACIYLAARTLEIPLPNRPHWFLLFGATEEEIQEICLKILQLYARKKVDLTHLEGEVEKRKHAIEEAKAQARGLLPGGTQVLDGTSGFSPAPKLVESPKEGKGSKPSPLSVKNTKRRLEGAKKAKADSPVNGLPKGRESRSRSRSREQSYSRSPSRSASPKRRKSDSGSTSGGSKSQSRSRSRSDSPPRQAPRSAPYKGSEIRGSRKSKDCKYPQKPHKSRSRSSSRSRSRSRERADNPGKYKKKSHYYRDQRRERSRSYERTGRRYERDHPGHSRHRR

SEQ ID NO: 19 - protein, 319 aa, Homo sapiens
MGIWQRLLLFGGVSLRAGGGATAPLGGSRAMVCGRQLSGAGSETLKQRRTQIMSRGLPKQKPIEGVKQVIVVASGKGGVGKSTTAVNLALALAANDSSKAIGLLDVDVYGPSVPKMMNLKGNPELSQSNLMRPLLNYGIACMSMGFLVEESEPVVWRGLMVMSAIEKLLRQVDWGQLDYLVVDMPPGTGDVQLSVSQNIPITGAVIVSTPQDIALMDAHKGAEMFRRVHVPVLGLVQNMSVFQCPKCKHKTHIFGADGARKLAQTLGLEVLGDIPLHLNIREASDTGQPIVFSQPESDEAKAYLRIAVEVVRRLPSPSE

SEQ ID NO: 20 - protein, 3926 aa, Homo sapiens
MGNEVSLEGGAGDGPLPPGGAGPGPGPGPGPGAGKPPSAPAGGGQLPAAGAARSTAVPPVPGPGPGPGPGPGPGSTSRRLDPKEPLGNQRAASPTPKQASATTPGHESPRETRAQGPAGQEADGPRRTLQVDSRTQRSGRSPSVSPDRGSTPTSPYSVPQIAPLPSSTLCPICKTSDLTSTPSQPNFNTCTQCHNKVCNQCGFNPNPHLTQVKEWLCLNCQMQRALGMDMTTAPRSKSQQQLHSPALSPAHSPAKQPLGKPDQERSRGPGGPQPGSRQAETARATSVPGPAQAAAPPEVGRVSPQPPQPTKPSTAEPRPPAGEAPAKSATAVPAGLGATEQTQEGLTGKLFGLGASLLTQASTLMSVQPEADTQGQPAPSKGTPKIVFNDASKEAGPKPLGSGPGPGPAPGAKTEPGARMGPGSGPGALPKTGGTTSPKHGRAEHQAASKAAAKPKTMPKERAICPLCQAELNVGSKSPANYNTCTTCRLQVCNLCGFNPTPHLVEKTEWLCLNCQTKRLLEGSLGEPTPLPPPTSQQPPVGAPHRASGTSPLKQKGPQGLGQPSGPLPAKASPLSTKASPLPSKASPQAKPLRASEPSKTPSSVQEKKTRVPTKAEPMPKPPPETTPTPATPKVKSGVRRAEPATPVVKAVPEAPKGGEAEDLVGKPYSQDASRSPQSLSDTGYSSDGISSSQSEITGVVQQEVEQLDSAGVTGPHPPSPSEIHKVGSSMRPLLQAQGLAPSERSKPLSSGTGEEQKQRPHSLSITPEAFDSDEELEDILEEDEDSAEWRRRREQQDTAESSDDFGSQLRHDYVEDSSEGGLSPLPPQPPARAAELTDEDFMRRQILEMSAEEDNLEEDDTATSGRGLAKHGTQKGGPRPRPEPSQEPAALPKRRLPHNATTGYEELLPEGGSAEATDGSGTLQGGLRRFKTIELNSTGSYGHELDLGQGPDPSLDREPELEMESLTGSPEDRSRGEHSSTLPASTPSYTSGTSPTSLSSLEEDSDSSPSRRQRLEEAKQQRKARHRSHGPLLPTIEDSSEEEELREEEELLREQEKMREVEQQRIRSTARKTRRDKEELRAQRRRERSKTPPSNLSPIEDASPTEELRQAAEMEELHRSSCSEYSPSPSLDSEAEALDGGPSRLYKSGSEYNLPTFMSLYSPTETPSGSSTTPSSGRPLKSAEEAYEEMMRKAELLQRQQGQAAGARGPHGGPSQPTGPRGLGSFEYQDTTDREYGQAAQPAAEGTPASLGAAVYEEILQTSQSIVRMRQASSRDLAFAEDKKKEKQFLNAESAYMDPMKQNGGPLTPGTSPTQLAAPVSFSTPTSSDSSGGRVIPDVRVTQHFAKETQDPLKLHSSPASPSSASKEIGMPFSQGPGTPATTAVAPCPAGLPRGYMTPASPAGSERSPSPSSTAHSYGHSPTTANYGSQTEDLPQAPSGLAAAGRAAREKPLSASDGEGGTPQPSRAYSYFASSSPPLSPSSPSESPTFSPGKMGPRATAEFSTQTPSPAPASDMPRSPGAPTPSPMVAQGTQTPHRPSTPRLVWQESSQEAPFMVITLASDASSQTRMVHASASTSPLCSPTETQPTTHGYSQTTPPSVSQLPPEPPGPPGFPRVPSAGADGPLALYGWGALPAENISLCRISSVPGTSRVEPGPRTPGTAVVDLRTAVKPTPIILTDQGMDLTSLAVEARKYGLALDPIPGRQSTAVQPLVINLNAQEHTFLATATTVSITMASSVFMAQQKQPVVYGDPYQSRLDFGQGGGSPVCLAQVKQVEQAVQTAPYRSGPRGRPREAKFARYNLPNQVAPLARRDVLITQMGTAQSIGLKPGPVPEPGAEPHRATPAELRSHALPGARKPHTVVVQMGEGTAGTVTTLLPEEPAGALDLTGMRPESQLACCDMVYKLPFGSSCTGTFHPAPSVPEKSMADAAPPGQSSSPFYGPRDPEPPEPPTYRAQGVVGPGPHEEQRPYPQGLPGRLYSSMSDTNLAEAGLNYHAQRIGQLFQGPGRDSAMDLSSLKHSYSLGFADGRYLGQGLQYGSVTDLRHPTDLLAHPLPMRRYSSVSNIYSDHRYGPRGDAVGFQEASLAQYSATTAREISRMCAALNSMDQYGGRHGSGGGGPDLVQYQPQHGPGLSAPQSLVPLRPGLLGNPTFPEGHPSPGNLAQYGPAAGQGTAVRQLLPSTATVRAADGMIYSTINTPIAATLPITTQPASVLRPMVRGGMYRPYASGGITAVPLTSLTRVPMIAPRVPLGPTGLYRYPAPSRFPIASSVPPAEGPVYLGKPAAAKAPGAGGPSRPEMPVGAAREEPLPTTTPAAIKEAAGAPAPAPLAGQKPPADAAPGGGSGALSRPGFEKEEASQEERQRKQQEQLLQLERERVELEKLRQLRLQEELERERVELQRHREEEQLLVQRELQELQTIKHHVLQQQQEERQAQFALQREQLAQQRLQLEQIQQLQQQLQQQLEEQKQRQKAPFPAACEAPGRGPPLAAAELAQNGQYWPPLTHAAFIAMAGPEGLGQPREPVLHRGLPSSASDMSLQTEEQWEASRSGIKKRHSMPRLRDACELESGTEPCVVRRIADSSVQTDDEDGESRYLLSRRRRARRSADCSVQTDDEDSAEWEQPVRRRRSRLPRHSDSGSDSKHDATASSSSAAATVRAMSSVGIQTISDCSVQTEPDQLPRVSPAIHITAATDPKVEIVRYISAPEKTGRGESLACQTEPDGQAQGVAGPQLVGPTAISPYLPGIQIVTPGPLGRFEKKKPDPLEIGYQAHLPPESLSQLVSRQPPKSPQVLYSPVSPLSPHRLLDTSFASSERLNKAHVSPQKHFTADSALRQQTLPRPMKTLQRSLSDPKPLSPTAEESAKERFSLYQHQGGLGSQVSALPPNSLVRKVKRTLPSPPPEEAHLPLAGQASPQLYAASLLQRGLTGPTTVPATKASLLRELDRDLRLVEHESTKLRKKQAELDEEEKEIDAKLKYLELGITQRKESLAKDRGGRDYPPLRGLGEHRDYLSDSELNQLRLQGCTTPAGQFVDFPATAAAPATPSGPTAFQQPRFQPPAPQYSAGSGGPTQNGFPAHQAPTYPGPSTYPAPAFPPGASYPAEPGLPNQQAFRPTGHYAGQTPMPTTQSTLFPVPADSRAPLQKPRQTSLADLEQKVPTNYEVIASPVVPMSSAPSETSYSGPAVSSGYEQGKVPEVPRAGDRGSVSQSPAPTYPSDSHYTSLEQNVPRNYVMIDDISELTKDSTSTAPDSQRLEPLGPGSSGRPGKEPGEPGVLDGPTLPCCYARGEEESEEDSYDPRGKGGHLRSMESNGRPASTHYYGDSDYRHGARVEKYGPGPMGPKHPSKSLAPAAISSKRSKHRKQGMEQKISKFSPIEEAKDVESDLASYPPPAVSSSLVSRGRKFQDEITYGLKKNVYEQQKYYGMSSRDAVEDDRIYGGSSRSRAPSAYSGEKLSSHDFSGWGKGYEREREAVERLQKAGPKPSSLSMAHSRVRPPMRSQASEEESPVSPLGRPRPAGGPLPPGGDTCPQFCSSHSMPDVQEHVKDGPRAHAYKREEGYILDDSHCVVSDSEAYHLGQEETDWFDKPRDARSDRFRHHGGHAVSSSSQKRGPARHSYHDYDEPPEEGLWPHDEGGPGRHASAKEHRHGDHGRHSGRHTGEEPGRRAAKPHARDLGRHEARPHSQPSSAPAMPKKGQPGYPSSAEYSQPSRASSAYHHASDSKKGSRQAHSGPAALQSKAEPQAQPQLQGRQAAPGPQQSQSPSSRQIPSGAASRQPQTQQQQQGLGLQPPQQALTQARLQQQSQPTTRGSAPAASQPAGKPQPGPSTATGPQPAGPPRAEQTNGSKGTAKAPQQGRAPQAQPAPGPGPAGVKAGARPGGTPGAPAGQPGADGESVFSKILPGGAAEQAGKLTEAVSAFGKKFSSFW

SEQ ID NO: 21 - protein, 213 aa, Homo sapiens [sequence omitted]

SEQ ID NO: 22 - protein, 601 aa, Homo sapiens
MAEPSGAETRPPIRVTVKTPKDKEEIVICDRASVKEFKEEISRRFKAQQDQLVLIFAGKILKDGDTLNQHGIKDGLTVHLVIKTPQKAQDPAAATASSPSTPDPASAPSTTPASPATPAQPSTSGSASSDAGSGSRRSSGGGPSPGAGEGSPSATASILSGFGGILGLGSLGLGSANFMELQQQMQRQLMSNPEMLSQIMENPLVQDMMSNPDLMRHMIMANPQMQQLMERNPEISHMLNNPELMRQTMELARNPAMMQEMMRNQDRALSNLESIPGGYNALRRMYTDIQEPMFSAAREQFGNNPFSSLAGNSDSSSSQPLRTENREPLPNPWSPSPPTSQAPGSGGEGTGGSGTSQVHPTVSNPFGINAASLGSGMFNSPEMQALLQQISENPQLMQNVISAPYMRSMMQTLAQNPDFAAQMMVNVPLFAGNPQLQEQLRLQLPVFLQQMQNPESLSILTNPRAMQALLQIQQGLQTLQTEAPGLVPSLGSFGISRTPAPSAGSNAGSTPEAPTSSPATPATSSPTGASSAQQQLMQQMIQLLAGSGNSQVQTPEVRFQQQLEQLNSMGFINREANLQALIATGGDINAAIERLLGSQLS

SEQ ID NO: 23 - protein, 271 aa, Homo sapiens
MEAAAEPGNLAGVRHIILVLSGKGGVGKSTISTELALALRHAGKKVGILDVDLCGPSIPRMLGAQGRAVHQCDRGWAPVFLDREQSISLMSVGFLLEKPDEAVVWRGPKKNALIKQFVSDVAWGELDYLVVDTPPGTSDEHMATIEALRPYQPLGALVVTTPQAVSVGDVRRELTFCRKTGLRVMGIVENMSGFTCPHCTECTSVFSRGGGEELAQLAGVPFLGSVPLDPALMRTLEEGHDFIQEFPGSPAFAALTSIAQKILDATPACLP

SEQ ID NO: 24 - protein, 258 aa, Homo sapiens
MPPQQGDPAFPDRCEAPPVPPRRERGGRGGRGPGEPGGRGRAGGAEGRGVKCVLVGDGAVGKTSLVVSYTTNGYPTEYIPTAFDNFSAVVSVDGRPVRLQLCDTAGQDEFDKLRPLCYTNTDIFLLCFSVVSPSSFQNVSEKWVPEIRCHCPKAPIILVGTQSDLREDVKVLIELDKCKEKPVPEEAAKLCAEEIKAASYIECSALTQKNLKEVFDAAIVAGIQYSDTQQQPKKSKSRTPDKMKNLSKSWWKKYCCFV

SEQ ID NO: 25 - protein, 1464 aa, Homo sapiens
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPTGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 26 - protein, 585 aa, Homo sapiens
MTPPRLFWVWLLVAGTQGVNDGDMRLADGGATNQGRVEIFYRGQWGTVCDNLWDLTDASVVCRALGFENATQALGRAAFGQGSGPIMLDEVQCTGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVD

SEQ ID NO: 27 - protein, 249 aa, Homo sapiens
MASRRMETKPVITCLKTLLIIYSFVFWITGVILLAVGVWGKLTLGTYISLIAENSTNAPYVLIGTGTTIVVFGLFGCFATCRGSPWMLKLYAMFLSLVFLAELVAGISGFVFRHEIKDTFLRTYTDAMQTYNGNDERSRAVDHVQRSLSCCGVQNYTNWSTSPYFLEHGIPPSCCMNETDCNPQDLHNLTVAATKVNQKGCYDLVTSFMETNMGIIAGVAFGIAFSQLIGMLLACCLSRFITANQYEMV

SEQ ID NO: 28 - protein, 218 aa, Homo sapiens
MGTRDDEYDYLFKVVLIGDSGVGKSNLLSRFTRNEFNLESKSTIGVEFATRSIQVDGKTIKAQIWDTAGQERYRAITSAYYRGAVGALLVYDIAKHLTYENVERWLKELRDHADSNIVIMLVGNKSDLRHLRAVPTDEARAFAEKNNLSFIETSALDSTNVEEAFKNILTEIYRIVSQKQIADRAAHDESPGNNVVDISVPPTTDGQKPNKLQCCQNL

SEQ ID NO: 29 - protein, 12 aa, synthetic construct
  REGION 1..12: note = 12-mer peptide selected from a combinatorial phage display library, wherein the peptide is expressed on the phage capsid and binds the BF774 monoclonal antibody, also referred to as mimotope.
HSFTTYLTVHPS

SEQ ID NO: 30 - protein, 12 aa, synthetic construct
  REGION 1..12: note = 12-mer peptide selected from a combinatorial phage display library, wherein the peptide is expressed on the phage capsid and binds the BF774 monoclonal antibody, also referred to as mimotope.
TYYPSSIPGFTL

SEQ ID NO: 31 - protein, 12 aa, synthetic construct
  REGION 1..12: note = 12-mer peptide selected from a combinatorial phage display library, wherein the peptide is expressed on the phage capsid and binds the BF774 monoclonal antibody, also referred to as mimotope.
HLTHSPIPVRAM